(19) United States
(12) Patent Application Publication
(10) Pub. No.: US 2025/0346581 A1
(43) Pub. Date: Nov. 13, 2025

(54) PROCESS FOR SYNTHESIS OF GALBULIMIMA ALKALOID 18 AND COMPOUNDS USEFUL AS OPIOID RECEPTOR ANTAGONISTS AND AGONISTS

(71) Applicants: The Scripps Research Institute, La Jolla, CA (US); University of Florida Research Foundation, Incorporated, Gainsville, FL (US)

(72) Inventors: Ryan Shenvi, San Diego, CA (US); Stone Woo, San Diego, CA (US); Laura Bohn, Jupiter, FL (US); Florian Zielke, Dortmund (DE)

(21) Appl. No.: 18/728,925

(22) PCT Filed: Jan. 20, 2023
(86) PCT No.: PCT/US23/61027

§ 371 Date: Jul. 15, 2024

Related U.S. Application Data
(60) Provisional application No. 63/301,677, filed on Jan. 21, 2022.

(51) Int. Cl. C07D 405/08; A61K 31/453; B01J 31/24

(52) U.S. Cl. CPC C07D 405/08; A61K 31/453; B01J 31/2404; C07B 2200/07

(57) ABSTRACT

The present invention provides a process for the synthesis of Galbulimima alkaloid 18 (GB18). Also provided are compounds that are useful as opioid receptor antagonists and agonists.

### CROSS-REFERENCE

This application claims the benefit of U.S. Provisional Application No. 63/301,677, filed on Jan. 21, 2022, which is incorporated herein by reference in its entirety.

### GOVERNMENTAL SUPPORT

This invention was made with government support under grant number R35GM122606 awarded by the National Institutes of Health and grant number CHE1856747 awarded by National Science Foundation. The government has certain rights in the invention.

### FIELD OF INVENTION

This invention provides to a process for preparing a racemic or scalemic mixture of Galbulimima alkaloid GB18, as well as compounds useful as opioid receptor antagonists and agonists.

### BACKGROUND OF THE DISCLOSURE

The GB alkaloids¹⁻³ derive from the bark of Galbulimima belgraveana, which features in the traditional medicine and ritual of Papua New Guinea as an analgesic, antipyretic and hallucinogen.⁴⁻⁷ Forty alkaloids unique to Galbulimima comprise four structural classes differentiated by connectivity between conserved piperidine and decalin motifs (Classes I-IV, see FIG. 1). Of twelve alkaloids subjected to in vivo assays, ten elicited physiological or behavior change in mammals at or below 10 mg/kg. Most affected heart rate, blood pressure or muscle spasm. The most potent antispasmotic, himbacine (Class I) garnered the most interest as a candidate for treatment of bradycardia (abnormally slow heart beat), 8 Alzheimer's disease⁹⁻¹¹ and intraocular pressure¹² due to its potent antagonism of muscarinic acetylcholine receptor (mAChR) M2 (K_{d}=4 nM).^{Error! Bookmark not defined.} Himandrine (Class II) induced marked and sustained hypotension in cats at 2.5 mg/kg (i.v. administration) and, opposite to the tachycardiac himbacine, reduced heart rate (induced bradycardia). SmithKlineFrench suggested himandrine may suppress sympathetic centers of the hypothalamus region of the brain. Himbadine effected significant antispasmodic activity at 0.1 mg/L in rabbit intestine (furmethide-induced spasm); himbeline was weakly depressant and hypotensive (no dose listed); 2.5 mg/kg of himandridine produced moderate to marked hypotensive activity with no indication of peripheral autonomic nervous system effects; and himandravine induced strong CNS depression and anticonvulsant activity against electroshock seizure.

Only a single alkaloid displayed activity consistent with psychotropic effects. Alkaloid J (GB18) inhibited mouse preening at 5 mg/kg, with no effect on pain threshold, suggesting an effect on cognition instead of sensation. A high potency target was not identified. GB18 is not widely available and its abundance in Galbulimima bark is not reported. However, the extreme variability of overall alkaloid content and ratio, unrelated to location and season (0.5% to trace total alkaloid content, avg. content 57 ppm, excluding the abundant alkaloid, himbacine)¹³ frustrated reisolation attempts, leading to ad hoc procedures for extraction and purification.¹⁴

There is therefore a need for a synthetic procedure for preparing GB18 that overcomes prior synthetic problems.

### SUMMARY OF THE DISCLOSURE

Some embodiments described herein provide a process for preparing a scalemic or racemic mixture of Galbulimima alkaloid GB18, comprising a mixture of the enantiomers of Formulae (1) and (2):

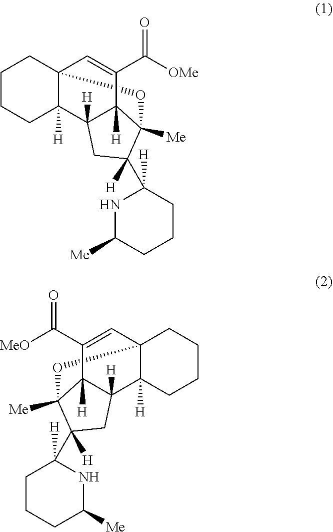 [SMILES: COC(=O)C1=C[C@]23CCCC[C@@H]2[C@H]2C[C@@H]([C@H]4CCC[C@@H](C)N4)[C@@](C)(O3)[C@@H]12 ; COC(=O)C1=C[C@@]23CCCC[C@H]2[C@@H]2C[C@H]([C@@H]4CCC[C@H](C)N4)[C@](C)(O3)[C@H]12]

the process comprising the sequential steps of:
(a) epoxidizing the compound of Formula (5)

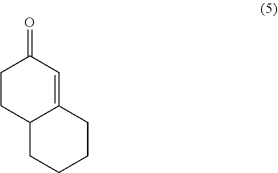 [SMILES: O=C1C=C2CCCCC2CC1]

to form the compound of Formula (SI-2)

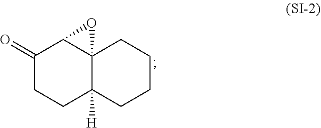 [SMILES: O=C1CC[C@@H]2CCCC[C@@]23O[C@@H]13]

(b) hydrogenating the compound of Formula (SI-2) to form the compound of Formula (6)

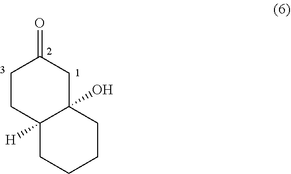 [SMILES: O=C1CC[C@@H]2CCCC[C@]2(O)C1]

(c) silylating the compound of Formula (6) to form the compound of Formula (SI-3)

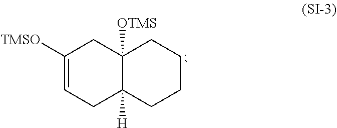

(d) conducting a Saegusa oxidation of the compound of Formula (SI-3) to form the compound of Formula (4)

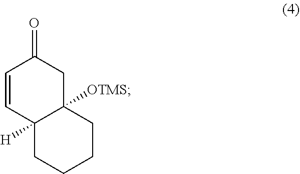

(e) reacting the compound of Formula (4) with the compound of Formula (SI-4)

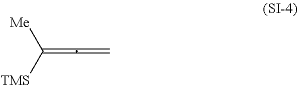

to form the compound of Formula (7)

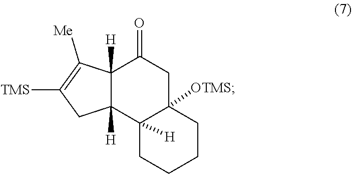

(f) desilylating the compound of Formula (7) to form the compound of Formula (8)

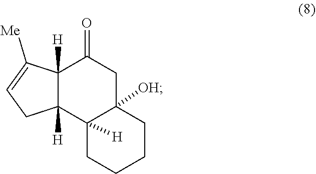

(g) iodoetherifying the compound of Formula (8) to form the compound of Formula (3)

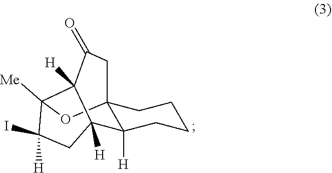 [SMILES: CC12OC34CCCCC3[C@H](C[C@H]1I)[C@H]2C(=O)C4]

(h) conducting a cross-electrophile coupling of the compound of Formula (3) with 2-iodo-6-methylpyridine (SI-6) to form the compound of Formula (2)

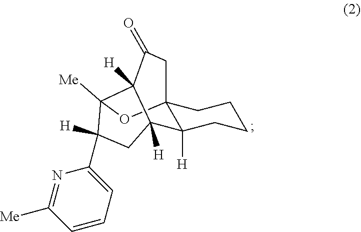

(i) conducting a pyridine N-oxidation of the compound of Formula (2) to form the compound of Formula (9)

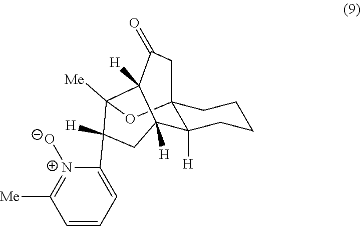 [SMILES: Cc1cccc([C@H]2C[C@H]3C4CCCCC45CC(=O)[C@H]3C2(C)O5)[n+]1[O-]]

(j) hydrogenating the compound of Formula (9) to form the compound of Formula (10)

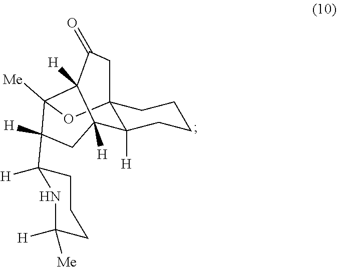 [SMILES: CC1CCCC([C@H]2C[C@H]3C4CCCCC45CC(=O)[C@H]3C2(C)O5)N1]

(k) condensing the compound of Formula (10) with hydrazine/hydrazine hydrate or a hydrazine derivative to form the hydrazone compound of Formula (SI-8)

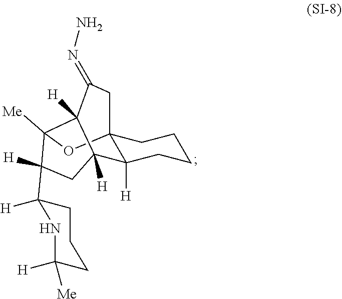 [SMILES: CC1CCCC([C@H]2C[C@H]3C4CCCCC45C/C(=N\N)[C@H]3C2(C)O5)N1]

(l) halogenating the compound of Formula (SI-8) to form the compound of Formula (SI-9)

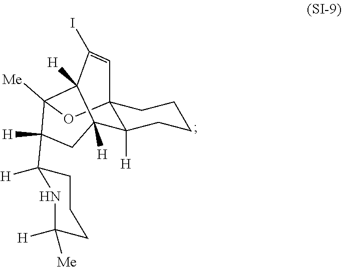 [SMILES: CC1CCCC([C@H]2C[C@H]3C4CCCCC45C=C(I)[C@H]3C2(C)O5)N1]

and
(m) conducting a carbonylation of the compound of Formula (SI-9) to form the scalemic or racemic mixture.

Some embodiments described herein also provide a process for preparing a scalemic or racemic mixture of Galbulimima alkaloid GB18, comprising a mixture of the enantiomers of Formulae (1) and (2):

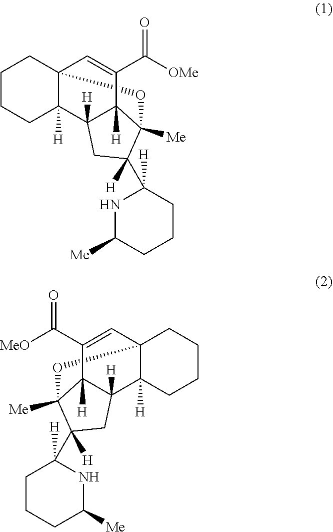 [SMILES: COC(=O)C1=C[C@]23CCCC[C@@H]2[C@H]2C[C@@H]([C@H]4CCC[C@@H](C)N4)[C@@](C)(O3)[C@@H]12 ; COC(=O)C1=C[C@@]23CCCC[C@H]2[C@@H]2C[C@H]([C@@H]4CCC[C@H](C)N4)[C@](C)(O3)[C@H]12]

the process comprising using at least one compound selected from the group consisting of:

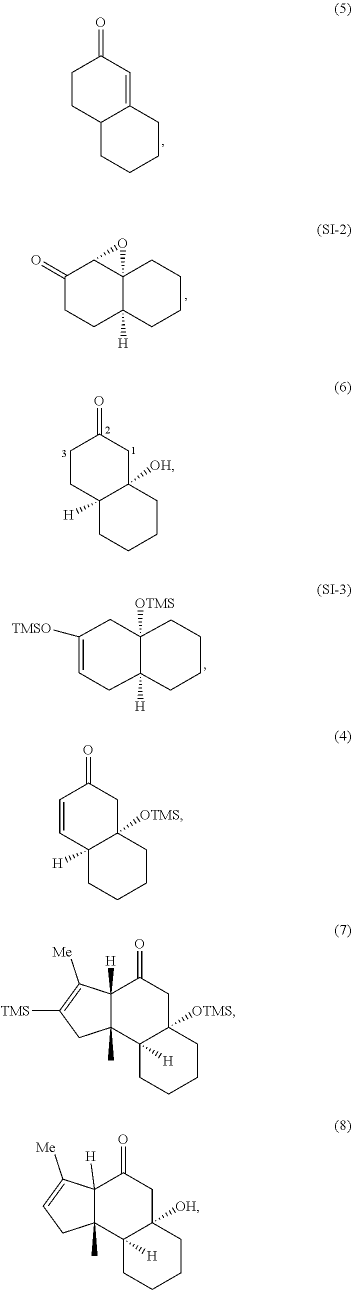

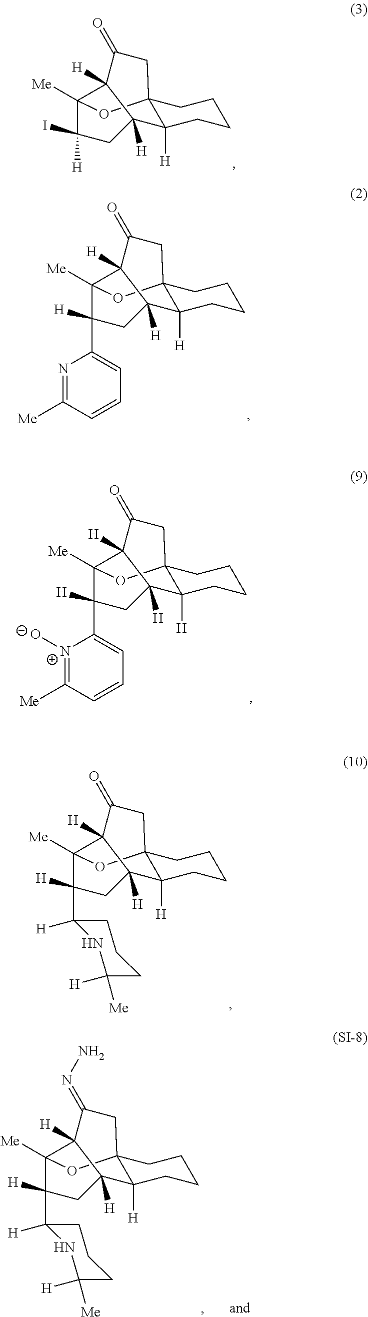 [SMILES: CC12OC34CCCCC3[C@H](C[C@H]1I)[C@H]2C(=O)C4 ; Cc1cccc([C@H]2C[C@H]3C4CCCCC45CC(=O)[C@H]3C2(C)O5)n1 ; Cc1cccc([C@H]2C[C@H]3C4CCCCC45CC(=O)[C@H]3C2(C)O5)[n+]1[O-] ; CC1CCCC([C@H]2C[C@H]3C4CCCCC45CC(=O)[C@H]3C2(C)O5)N1 ; CC1CCCC([C@H]2C[C@H]3C4CCCCC45C/C(=N\N)[C@H]3C2(C)O5)N1]

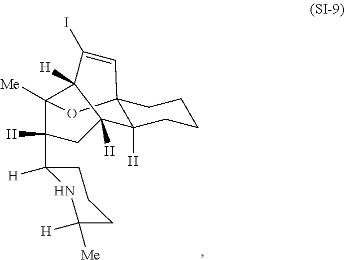 [SMILES: CC1CCCC([C@H]2C[C@H]3C4CCCCC45C=C(I)[C@H]3C2(C)O5)N1]

or an enantiomer, a scalemic mixture or a racemic mixture thereof, as an intermediate in the preparation of the scalemic or racemic mixture of Galbulimima alkaloid GB18.

Some embodiments described herein also provide a compound selected from the group consisting of:

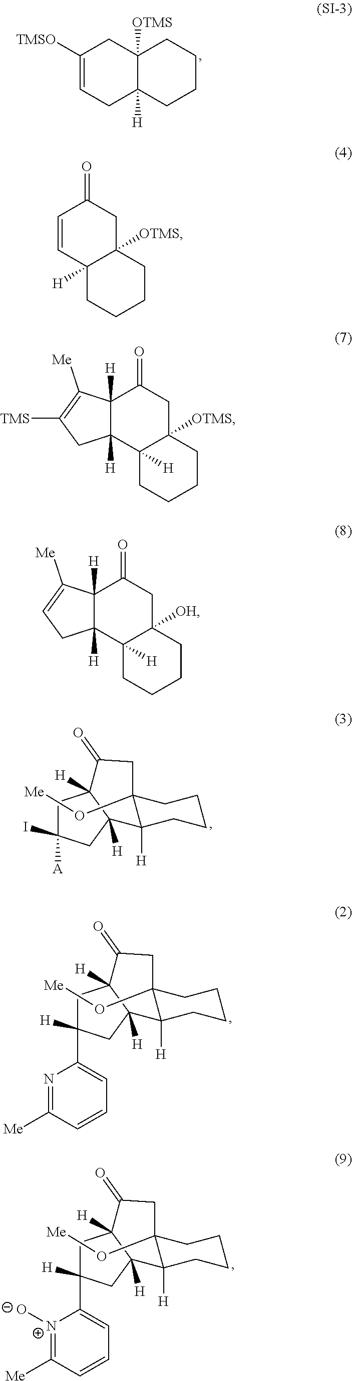

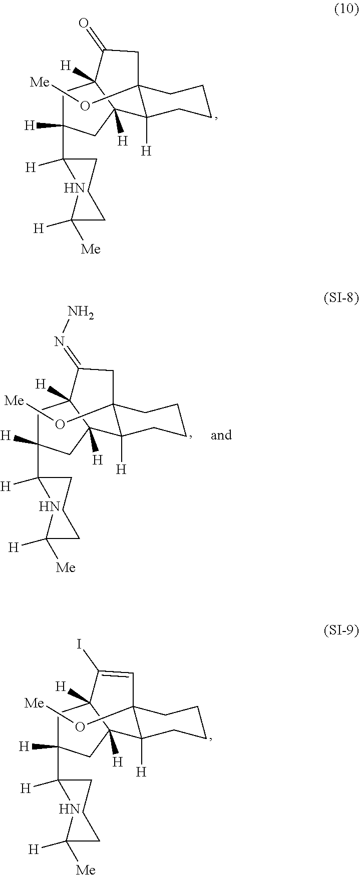

or an enantiomer, a scalemic mixture or a racemic mixture thereof.

Some embodiments described herein also provide a compound of Formula (1)

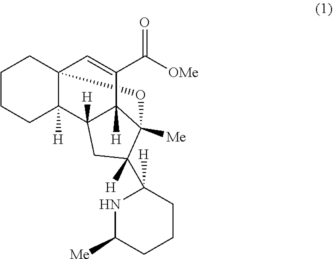 [SMILES: COC(=O)C1=C[C@]23CCCC[C@@H]2[C@H]2C[C@@H]([C@H]4CCC[C@@H](C)N4)[C@@](C)(O3)[C@@H]12]

or a pharmaceutically acceptable salt thereof.

Some embodiments described herein also provide a method of antagonizing an opioid receptor in a subject in need of such antagonization, comprising administering to such subject a therapeutically effective amount of a compound of Formula (I)

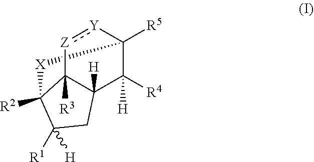

wherein:
[symbol] is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷;
   or R⁶ and R⁷ together form monocyclic or bicyclic C₃-C₁₄ cycloalkyl or C₃-C₁₄ heterocycloalkyl, each individually and optionally substituted with one or more R when [symbol] is a single bond;
   or R⁶ and R⁷ together form monocyclic or bicyclic C₅-C₁₄ heteroaryl optionally substituted with one or more R when [symbol] is a double bond;
Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
   each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇cycloalkyl), —NMsPh, and —C(═O)OH;
R² and R³ are independently H, C₁-C₆ alkyl, (C₁-C₆ alkyl)-C(═O)—, hydroxy, or —O—(C₁-C₆ alkyl);
   or R² and R³ together form C₃-C₇ cycloalkyl or C₃-C₇ heterocycloalkyl;
R⁴ and R⁵ are each independently H or C₁-C₆ alkyl;
   or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
   or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
   or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
   or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl); or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
each R^{p} independently is H, C₁-C₆ alkyl, —C(═O)—(C₁-C₆ alkyl), —Si(C₁-C₆ alkyl)₃, —S(═O)₂—(C₁-C₆ alkyl), —S(═O)₂—(C₆-C₁₀ aryl), or —NHTs;
R^{q}, R^{s}, and R^{t} are each independently H or C₁-C₆ alkyl;
   wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
   wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
   or a pharmaceutically acceptable salt thereof.

Some embodiments described herein also provide a method of treating a disorder selected from the group consisting of substance abuse disorder, major depressive disorder, resistant depression, and impulse control disorder in a subject in need of such treatment comprising administering to the subject a therapeutically effective amount of a compound of Formula (I)

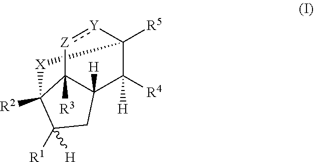

wherein:
[symbol] is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷;
   or R⁶ and R⁷ together form monocyclic or bicyclic C₃-C₁₄ cycloalkyl or C₃-C₁₄ heterocycloalkyl, each individually and optionally substituted with one or more R when [symbol] is a single bond;
   or R⁶ and R⁷ together form monocyclic or bicyclic C₅-C₁₄ heteroaryl optionally substituted with one or more R when [symbol] is a double bond;
Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇ cycloalkyl), —NMsPh, and —C(═O)OH;
R² and R³ are independently H, C₁-C₆ alkyl, (C₁-C₆ alkyl)-C(═O)—, hydroxy, or —O—(C₁-C₆ alkyl);
   or R² and R³ together form C₃-C₇ cycloalkyl or C₃-C₇ heterocycloalkyl;
R⁴ and R⁵ are each independently H or C₁-C₆ alkyl;
   or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
   or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
   or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
   or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl); or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
each R^{p} independently is H, C₁-C₆ alkyl, —C(═O)—(C₁-C₆ alkyl), —Si(C₁-C₆ alkyl)₃, —S(═O)₂—(C₁-C₆ alkyl), —S(═O)₂—(C₆-C₁₀ aryl), or —NHTs;
R^{q}, R^{s}, and R^{t} are each independently H or C₁-C₆ alkyl;
   wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
   wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
   or a pharmaceutically acceptable salt thereof.

Some embodiments described herein also provide a method of agonizing an opioid receptor in a subject in need of such agonization, comprising administering to the subject a therapeutically effective amount of the compound of Formula (II):

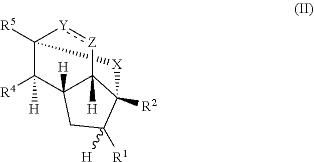

wherein:
[symbol] is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷;
   or R⁶ and R⁷ together form monocyclic or bicyclic C₃-C₁₄ cycloalkyl or C₃-C₁₄ heterocycloalkyl, each individually and optionally substituted with one or more R when [symbol] is a single bond;
   or R⁶ and R⁷ together form monocyclic or bicyclic C₅-C₁₄ heteroaryl optionally substituted with one or more R when [symbol] is a double bond;
Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
   each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇ cycloalkyl), —NMsPh, and —C(═O)OH;
R² and R³ are independently H, C₁-C₆ alkyl, (C₁-C₆ alkyl)-C(═O)—, hydroxy, or —O—(C₁-C₆ alkyl);
   or R² and R³ together form C₃-C₇ cycloalkyl or C₃-C₇ heterocycloalkyl;
R⁴ and R⁵ are each independently H or C₁-C₆ alkyl;
   or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
   or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
   or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
   or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl); or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
each R^{p} independently is H, C₁-C₆ alkyl, —C(═O)—(C₁-C₆ alkyl), —Si(C₁-C₆ alkyl)₃, —S(═O)₂—(C₁-C₆ alkyl), —S(═O)₂—(C₆-C₁₀ aryl), or —NHTs;
R^{q}, R^{s}, and R^{t} are each independently H or C₁-C₆ alkyl;
   wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
   wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
   or a pharmaceutically acceptable salt thereof.

Some embodiments described herein also provide a method of treating pain, itching, depression or dissociative hallucination in a subject in need of such treatment comprising administering to the subject a therapeutically effective amount of the compound of Formula (II):

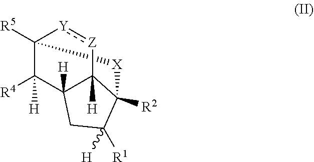

wherein:
[symbol] is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷;
   or R⁶ and R⁷ together form monocyclic or bicyclic C₃-C₁₄ cycloalkyl or C₃-C₁₄ heterocycloalkyl, each individually and optionally substituted with one or more R when [symbol] is a single bond;
   or R⁶ and R⁷ together form monocyclic or bicyclic C₅-C₁₄ heteroaryl optionally substituted with one or more R when [symbol] is a double bond;
Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
   each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH—CH₂, (C₁-C₆ alkyl)(C₃-C₇cycloalkyl), —NMsPh, and —C(═O)OH;
R² and R³ are independently H, C₁-C₆ alkyl, (C₁-C₆ alkyl)-C(═O)—, hydroxy, or —O—(C₁-C₆ alkyl);
   or R² and R³ together form C₃-C₇ cycloalkyl or C₃-C₇ heterocycloalkyl;
R⁴ and R⁵ are each independently H or C₁-C₆ alkyl;
   or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
   or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
   or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
   or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl); or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
each R^{p} independently is H, C₁-C₆ alkyl, —C(═O)—(C₁-C₆ alkyl), —Si(C₁-C₆ alkyl)₃, —S(═O)₂—(C₁-C₆ alkyl), —S(═O)₂—(C₆-C₁₀ aryl), or —NHTs;
R^{q}, R^{s}, and R^{t} are each independently H or C₁-C₆ alkyl;
   wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
   wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
   or a pharmaceutically acceptable salt thereof.

The application further provides A compound of formula I, having the structure of any one of the group consisting of:

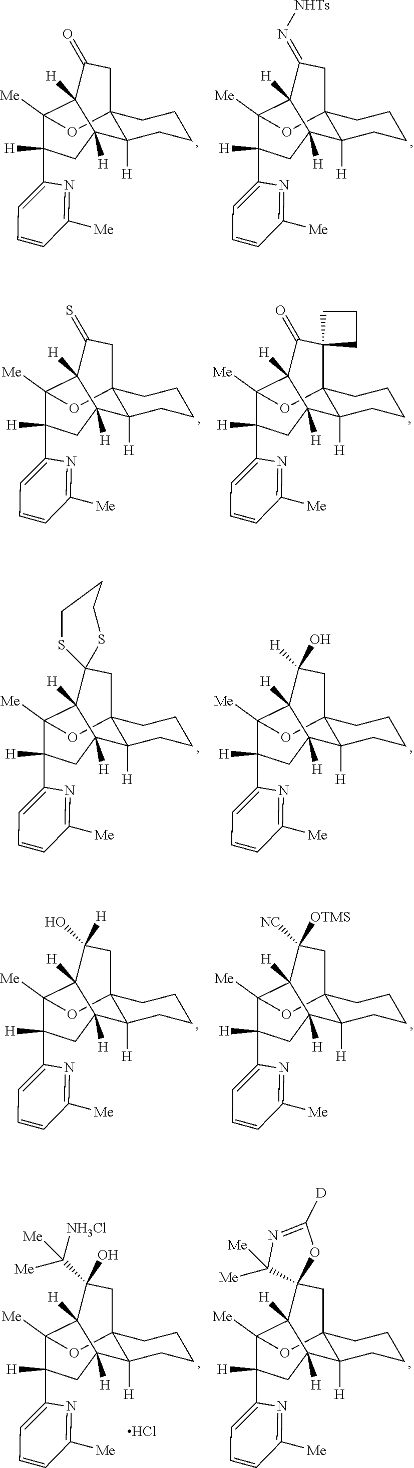

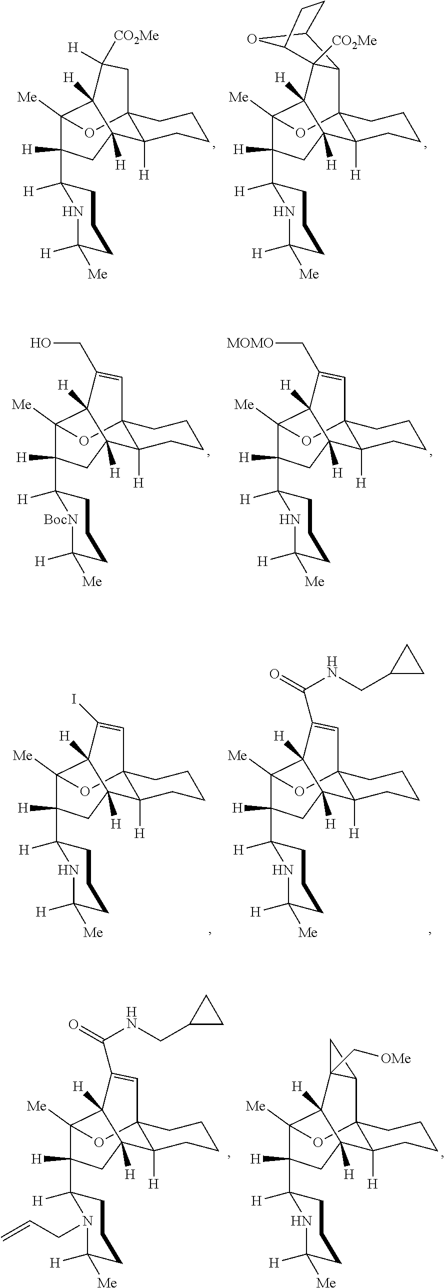

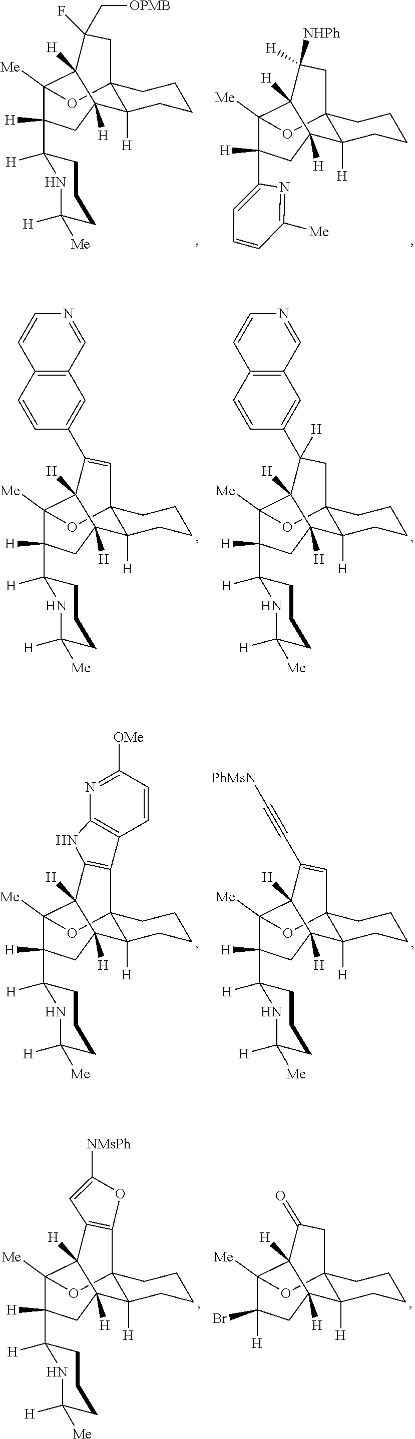

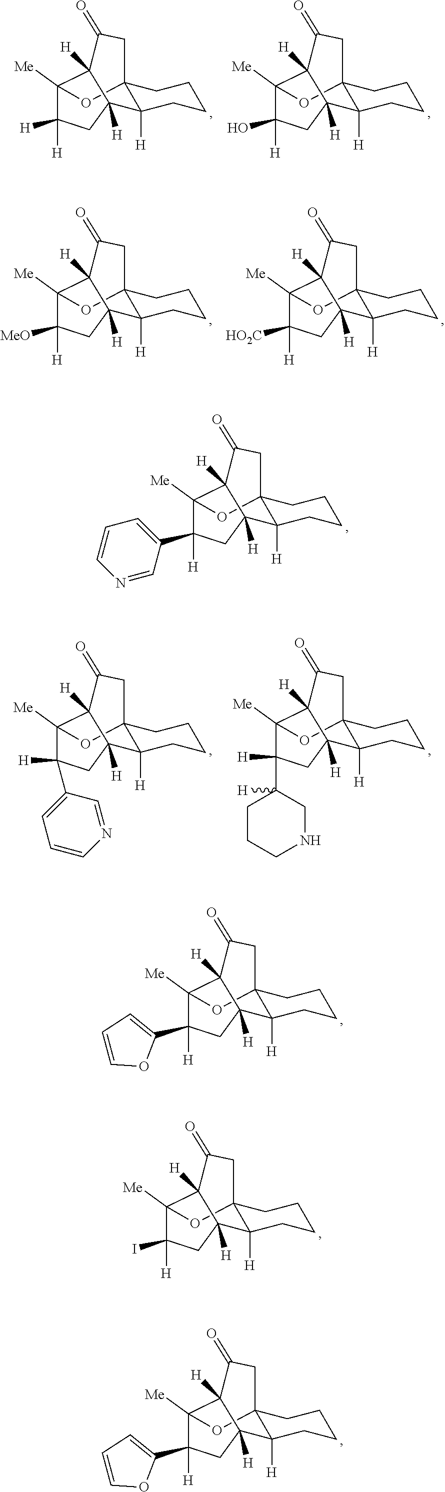

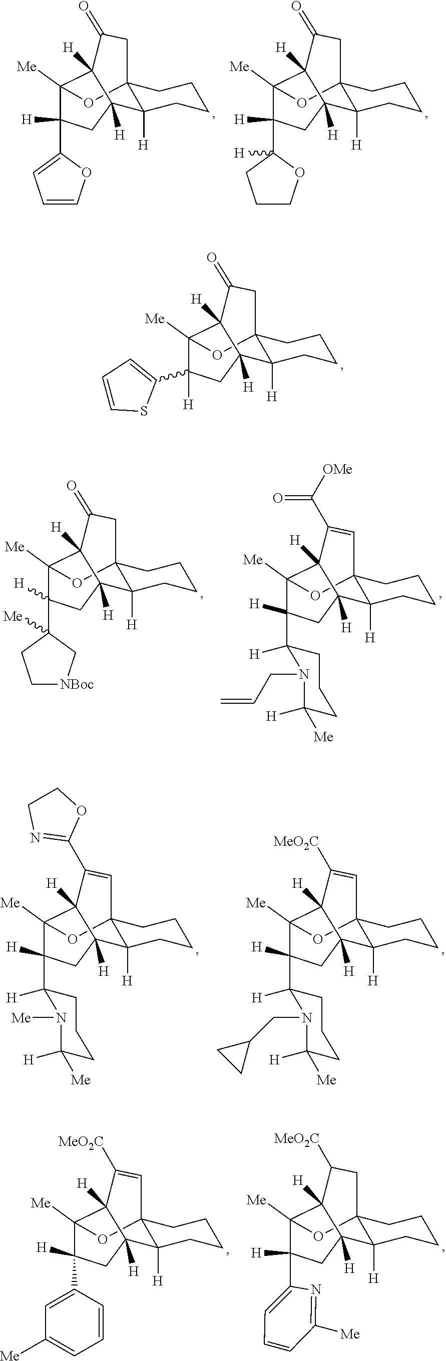

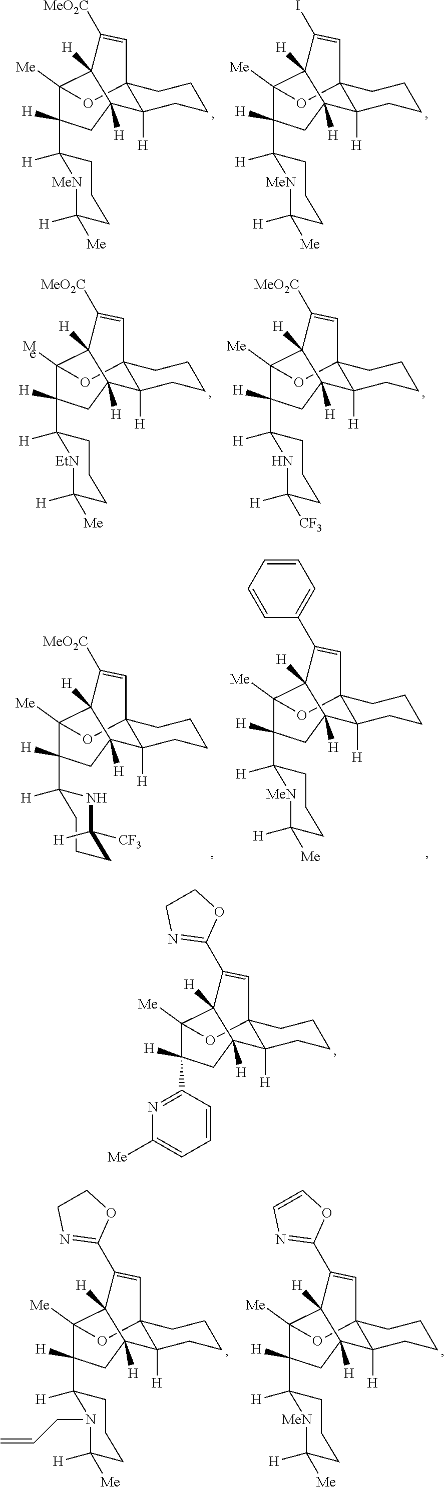

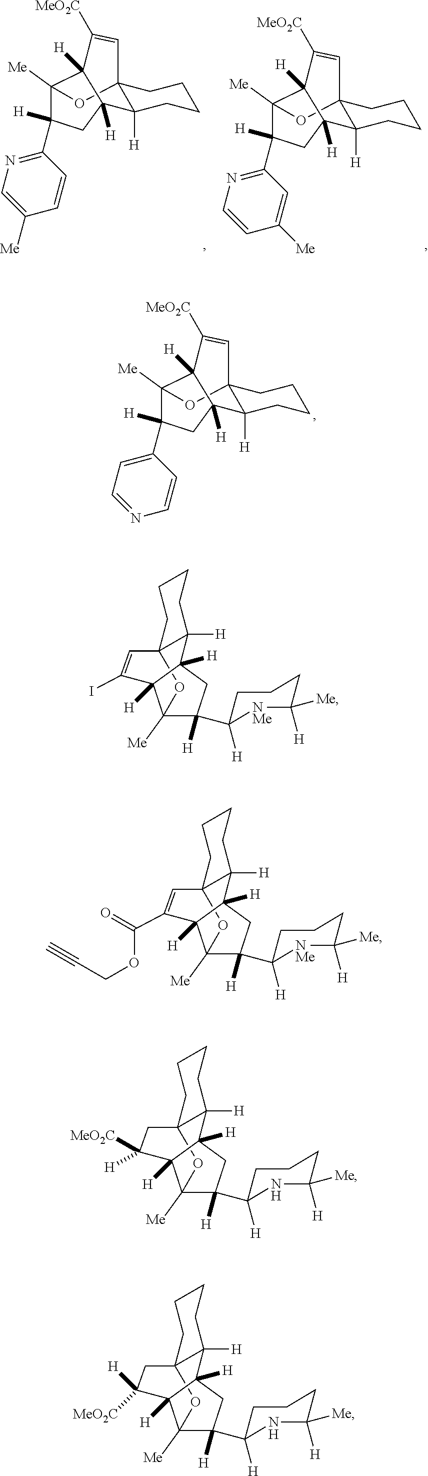

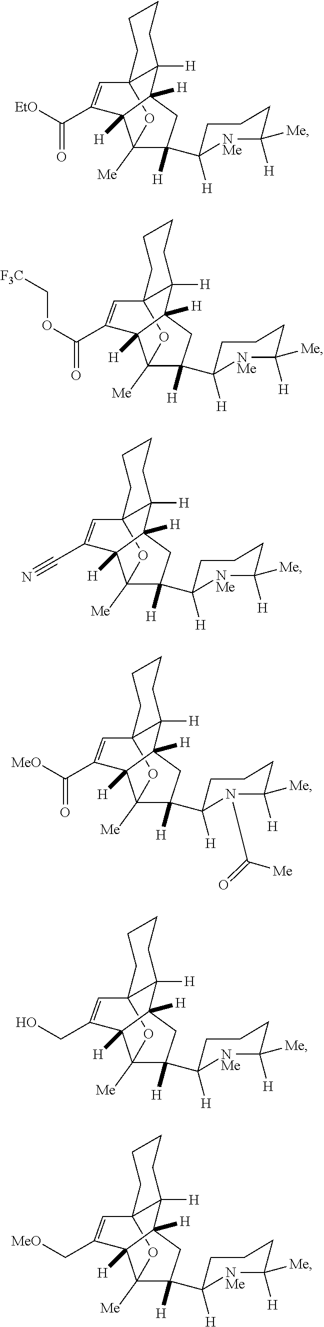

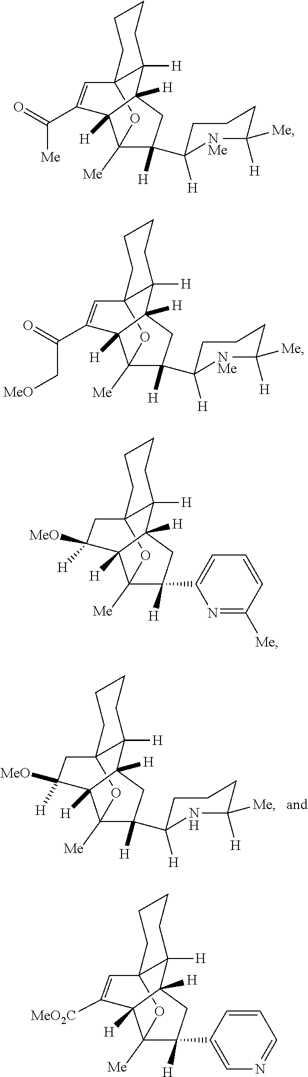

including any enantiomers, scalemic or racemic mixtures, and pharmaceutically acceptable salts thereof.

### BRIEF DESCRIPTION OF THE FIGURES

FIG. 1 shows that GB alkaloids are classified by piperidine/decalin topology and exhibit diverse nervous-system effects in vivo, but a target has only been assigned to the most abundant alkaloid, himbacine.

FIG. 2 shows that stereoselective attached-ring cross-coupling allows a short synthesis of GB18 (1).

FIG. 3 shows that Class I alkaloid GB18 (single, naturally-occurring enantiomer) antagonizes opioid receptors with high potency for kappa- and mu- (KOR and MOR) but does not bind M1-5, in contrast to Class I alkaloid himbacine. Thus, the topology changes that differentiate the two subclasses also differentiate target selectivity.

FIG. 4 shows the crystal structure of slow-moving enantiomer ent-1, assigned as ent-GB18.

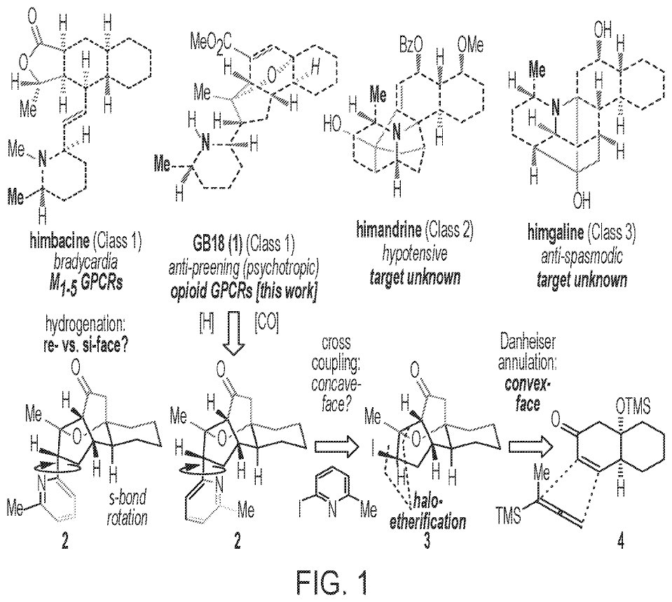

### DETAILED DESCRIPTION OF THE DISCLOSURE

Unless otherwise stated, the following terms used in the specification and claims are defined for the purposes of this Application and have the following meaning. All undefined technical and scientific terms used in this Application have the meaning as commonly understood by one of ordinary skill in the art to which this disclosure belongs.

As used herein, “a” or “an” entity refers to one or more of that entity; for example, a compound refers to one or more compounds or at least one compound unless stated otherwise. As such, the terms “a” (or “an”), “one or more”, and “at least one” can be used interchangeably herein.

“Patient” includes both human and animals. “Patient” and “subject” are used interchangeably herein.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds)(“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂-7 alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

In some embodiments, “carbocyclyl” is a monocyclic, saturated carbocyclyl group having from 3 to 14 ring carbon atoms (“C₃₋₁₄ cycloalkyl”). In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈).

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents may be selected from the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb}) OR^{aa}, —OC(═NR^{bb})R^{aa}, —OC(═NR^{bb}) OR^{aa}, —C(═NR^{bb})N(R^{bb})₂, —OC(═NR^{bb})N(R^{bb})₂, —NR^{bb}C(═NR^{bb})N(R^{bb})₂, —C(═O) NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O;
each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of Rec is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two Rec groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and
each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂ (C₁₋₆ alkyl), —OC(═O)(C₁₋₆ alkyl), —OCO₂ (C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH) OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —C₁), bromine (bromo, —Br), or iodine (iodo, —I).

It should be noted that in hetero-atom containing ring systems described herein, there are no hydroxyl groups on carbon atoms adjacent to a N, O or S, as well as there are no N or S groups on carbon adjacent to another heteroatom. Thus, for example, in the ring:

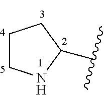 [SMILES: *C1CCCN1]

there is no-OH attached directly to carbons marked 2 and 5.

It should also be noted that tautomeric forms such as, for example, the moieties:

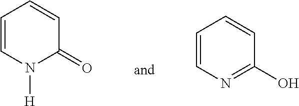 [SMILES: O=c1cccc[nH]1 ; Oc1ccccn1]

are considered equivalent unless otherwise specified.

As used herein, the term “composition” is intended to encompass a product comprising the specified ingredients in the specified amounts, as well as any product which results, directly or indirectly, from combination of the specified ingredients in the specified amounts.

As used herein, the term “scalemic mixture” refers to a nonracemic mixture of enantiomers containing unequal amounts of dextrorotatory (d) and levorotatory (l) stereoisomers (enantiomers). It contains an excess of one enantiomer over the other.

As used herein, the term “racemic mixture” or “racemate” refers to a mixture containing equal amounts of dextrorotatory (d) and levorotatory (l) stereoisomers (enantiomers) and therefore such a mixture is not optically active.

“Effective amount” or “therapeutically effective amount” is meant to describe an amount of compound or a composition described herein that is effective in inhibiting the above-noted enzyme, diseases or conditions, and thus producing the desired therapeutic, ameliorative, inhibitory and/or preventative effect.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., 14° C.) isotopes are particularly preferred for their ease of preparation and detectability. Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

The compounds described herein can also be used in combination with one or more additional therapeutic and/or prophylactic agents.

It is also possible to combine any compound of the invention with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound of the invention with one or more other active therapeutic agents generally refers to simultaneous or sequential administration of a compound of the invention and one or more other active therapeutic agents, such that therapeutically effective amounts of the compound of the invention and one or more other active therapeutic agents are both present in the body of the patient.

Co-administration includes administration of unit dosages of the compounds of the invention before or after administration of unit dosages of one or more other active therapeutic agents, for example, administration of the compounds of the invention within seconds, minutes, or hours of the administration of one or more other active therapeutic agents. For example, a unit dose of a compound of the invention can be administered first, followed within seconds or minutes by administration of a unit dose of one or more other active therapeutic agents. Alternatively, a unit dose of one or more other therapeutic agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound of the invention.

The combination therapy may provide “synergy” and “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-viral effect denotes an antiviral effect which is greater than the predicted purely additive effects of the individual compounds of the combination.

### EMBODIMENTS

Examples of embodiments of the present application include the following:

### Embodiment 1

A process for preparing a scalemic or racemic mixture of Galbulimima alkaloid GB18, comprising a mixture of the enantiomers of Formulae (1) and (2):

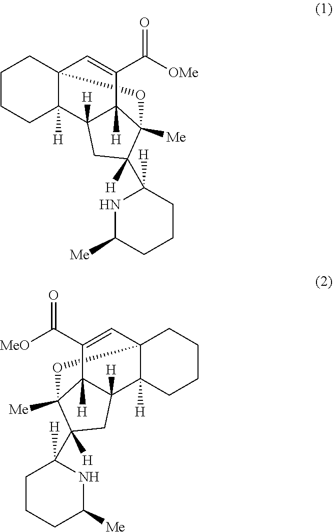 [SMILES: COC(=O)C1=C[C@]23CCCC[C@@H]2[C@H]2C[C@@H]([C@H]4CCC[C@@H](C)N4)[C@@](C)(O3)[C@@H]12 ; COC(=O)C1=C[C@@]23CCCC[C@H]2[C@@H]2C[C@H]([C@@H]4CCC[C@H](C)N4)[C@](C)(O3)[C@H]12]

the process comprising the sequential steps of:
(a) epoxidizing the compound of Formula (5)

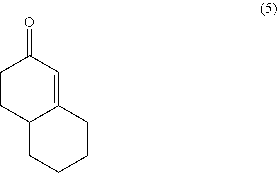 [SMILES: O=C1C=C2CCCCC2CC1]

to form the compound of Formula (SI-2)

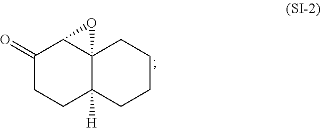 [SMILES: O=C1CC[C@@H]2CCCC[C@@]23O[C@@H]13]

(b) hydrogenating the compound of Formula (SI-2) to form the compound of Formula (6)

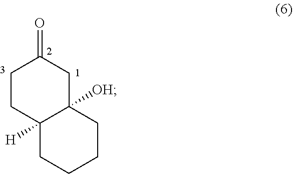 [SMILES: O=C1CC[C@@H]2CCCC[C@]2(O)C1]

(c) silylating the compound of Formula (6) to form the compound of Formula (SI-3)

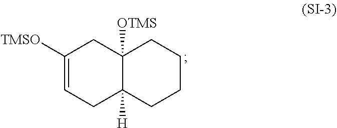

(d) conducting a Saegusa oxidation of the compound of Formula (SI-3) to form the compound of Formula (4)

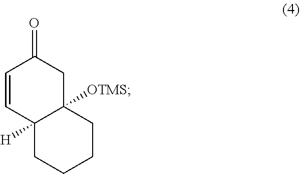

(e) reacting the compound of Formula (4) with the compound of Formula (SI-4)

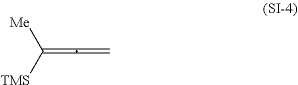

to form the compound of Formula (7)

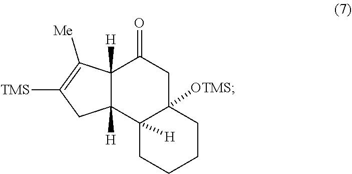

(f) desilylating the compound of Formula (7) to form the compound of Formula (8)

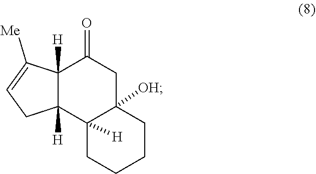

(g) iodoetherifying the compound of Formula (8) to form the compound of Formula (3)

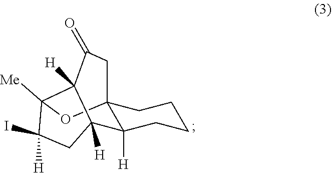 [SMILES: CC12OC34CCCCC3[C@H](C[C@H]1I)[C@H]2C(=O)C4]

(h) conducting a cross-electrophile coupling of the compound of Formula (3) with 2-iodo-6-methylpyridine (SI-6) to form the compound of Formula (2)

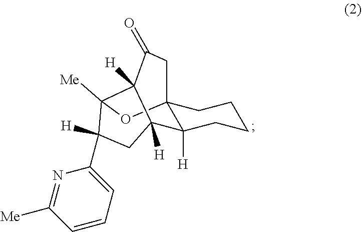 [SMILES: Cc1cccc([C@H]2C[C@H]3C4CCCCC45CC(=O)[C@H]3C2(C)O5)n1]

(i) conducting a pyridine N-oxidation of the compound of Formula (2) to form the compound of Formula (9)

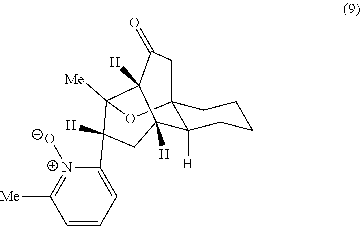 [SMILES: Cc1cccc([C@H]2C[C@H]3C4CCCCC45CC(=O)[C@H]3C2(C)O5)[n+]1[O-]]

(j) hydrogenating the compound of Formula (9) to form the compound of Formula (10)

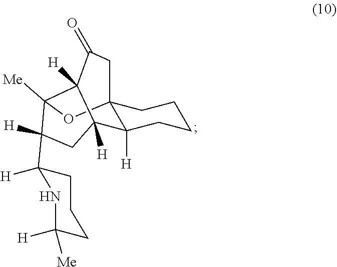 [SMILES: CC1CCCC([C@H]2C[C@H]3C4CCCCC45CC(=O)[C@H]3C2(C)O5)N1]

(k) condensing the compound of Formula (10) with hydrazine/hydrazine hydrate or a hydrazine derivative to form the hydrazone compound of Formula (SI-8)

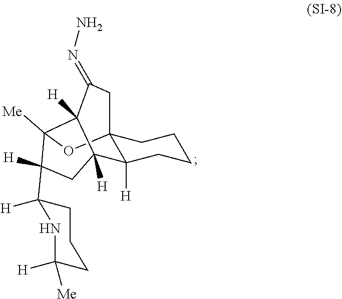 [SMILES: CC1CCCC([C@H]2C[C@H]3C4CCCCC45C/C(=N\N)[C@H]3C2(C)O5)N1]

(l) halogenating the compound of Formula (SI-8) to form the compound of Formula (SI-9)

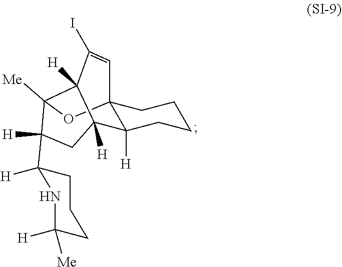 [SMILES: CC1CCCC([C@H]2C[C@H]3C4CCCCC45C=C(I)[C@H]3C2(C)O5)N1]

and
(m) conducting a carbonylation of the compound of Formula (SI-9) to form the scalemic or racemic mixture.

### Embodiment 2

The process according to Embodiment 1, wherein in step (a), the epoxidation of the compound of Formula (SI-2) is carried out in the presence of a peroxide, a peroxyacid reagent, or derivatives thereof. One skilled in the art will know the us of such epoxidation agents. Suitable nonlimiting examples of such reagents include m-chloroperbenzoic acid, tert-hydroperoxide, and urea-hydrogen complex.

### Embodiment 3

The process according to Embodiment 1 or 2, wherein in step (b), the hydrogenation of the compound of Formula (SI-2) is a metal-catalyzed hydrogenation.

### Embodiment 4

The process according to Embodiment 3, wherein the metal-catalyzed hydrogenation is conducted in the presence of ingredients comprising Pd/C and hydrogen gas.

### Embodiment 5

The process according to Embodiment 4, wherein the ingredients further comprise at least one solvent selected from the group consisting of 1,1,1,3,3,3-hexafluoro-2-propanol (HFIP) and ethyl acetate.

### Embodiment 6

The process according to Embodiment 5, wherein in step (c), the silylation of the compound of Formula (6) is carried out in the presence of at least one silylating agent selected from the group consisting of trimethylsilyl triflate, a halotrimethyl silane (such as chrotrimethylsilane, bromotrimethylsilane, and iodotrimethylsilane), hexamethyldisilazane, N,O-bis(trimethylsilyl) acetamide, N,O-bis(trimethylsilyl)trifluoroacetamide, and N,O-bis(trimethylsilyl) carbamate.

### Embodiment 7

The process according to any one of Embodiments 1-6, wherein in step (d), the Saegusa oxidation is carried out in the presence of a palladium salt and oxygen gas, or in the presence of an organic oxidizing agent.

### Embodiment 8

The process according to Embodiment 7, wherein the palladium salt is at least one selected from the group consisting of Pd(OAc)₂, or PdCl₂, and the organic oxidizing agent is 2-iodoxybenzoic acid.

### Embodiment 9

The process according to any one of Embodiments 1-8, wherein in step (e), the reaction of the compound of Formula (4) with the compound of Formula (SI-4) is carried out in the presence of a Lewis acid.

### Embodiment 10

The process according to Embodiment 9, wherein the Lewis acid is TiCl₄.

### Embodiment 11

The process according to any one of Embodiments 1-10, wherein in step (f), the desilylation of the compound of Formula (7) to form the compound of Formula (8) is carried out in the presence of at least one solvent comprising hexafluoroisopropanol (HFIP).

### Embodiment 12

The process according to Embodiment 11, further comprising recrystallizing a crude compound of Formula (8) formed from the desilylation of the compound of Formula (7).

### Embodiment 13

The process according to any one of Embodiments 1-10, wherein in step (g), the iodoetherification of the compound of Formula (8) is carried out in the presence of an electrophilic iodine reagent.

### Embodiment 14

The process according to Embodiment 13, wherein the electrophilic iodine reagent is N-iodosuccinimide.

### Embodiment 15

The process according to any one of Embodiments 1-14, wherein in step (h), the cross-electrophile coupling of the compound of Formula (3) with 2-iodo-6-methylpyridine (SI-6) is carried out in the presence of a ligand or a salt thereof.

### Embodiment 16

The process according to Embodiment 15, wherein the ligand or ligand salt is 1H-pyrazole-1-carboxamidine hydrochloride.

### Embodiment 17

The process according to any one of Embodiments 1-12, wherein in step (i), the pyridine N-oxidation of the compound of Formula (2) is carried out in the presence of meta-Chloroperbenzoic acid or methyltrioxorhenium/hydrogen peroxide.

### Embodiment 18

The process according to any one of Embodiments 1-13, wherein in step (j), the hydrogenation of the compound of Formula (9) is a metal catalyzed hydrogenation.

### Embodiment 19

The process according to Embodiment 18, wherein metal catalyzed hydrogenation is carried out in the presence of Rhodium/Al₂O₃ and hydrogen gas.

### Embodiment 20

The process according to any one of Embodiments 1-19, wherein in step (k), the compound of Formula (10) is condensed with hydrazine hydrate.

### Embodiment 21

The process according to any one of Embodiments 1-19, wherein wherein in step (1), the halogenation of the compound of Formula (SI-8) is iodination.

### Embodiment 22

The process according to Embodiment 21, wherein the iodination is carried out in the presence of a solution of iodine.

### Embodiment 23

The process according to any one of Embodiments 1-22, wherein in step (m), the carbonylation is a metal catalyzed carbonylation.

### Embodiment 24

The process according to Embodiment 23, wherein the metal catalyzed carbonylation is carried out in the presence of palladium catalyst(s), carbon monoxide gas, and methanol.

### Embodiment 25

The process according to Embodiment 24, wherein the palladium catalyst is selected from the group consisting of Pd(OAc)₂/PPh₃, Pd₂dba₃ (dibenzylidene acetone) Pd(PPh₃)₄, and Pd(MeCN)₂Cl₂.

### Embodiment 26

The process according to any one of Embodiments 1-25, further comprising resolving the scalemic or racemic mixture of GB18 into the enantiomers of Formulae (1) and (2).

### Embodiment 27

A process for preparing a scalemic or racemic mixture of Galbulimima alkaloid GB18, comprising a mixture of the enantiomers of Formulae (1) and (2):

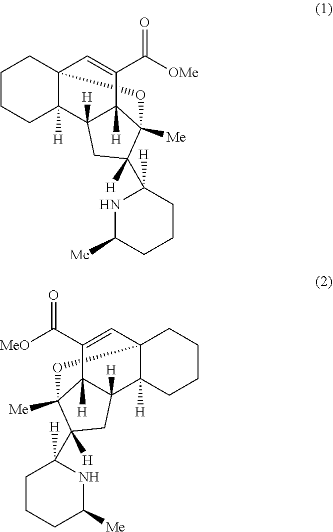 [SMILES: COC(=O)C1=C[C@]23CCCC[C@@H]2[C@H]2C[C@@H]([C@H]4CCC[C@@H](C)N4)[C@@](C)(O3)[C@@H]12 ; COC(=O)C1=C[C@@]23CCCC[C@H]2[C@@H]2C[C@H]([C@@H]4CCC[C@H](C)N4)[C@](C)(O3)[C@H]12]

the process comprising using at least one compound selected from the group consisting of:

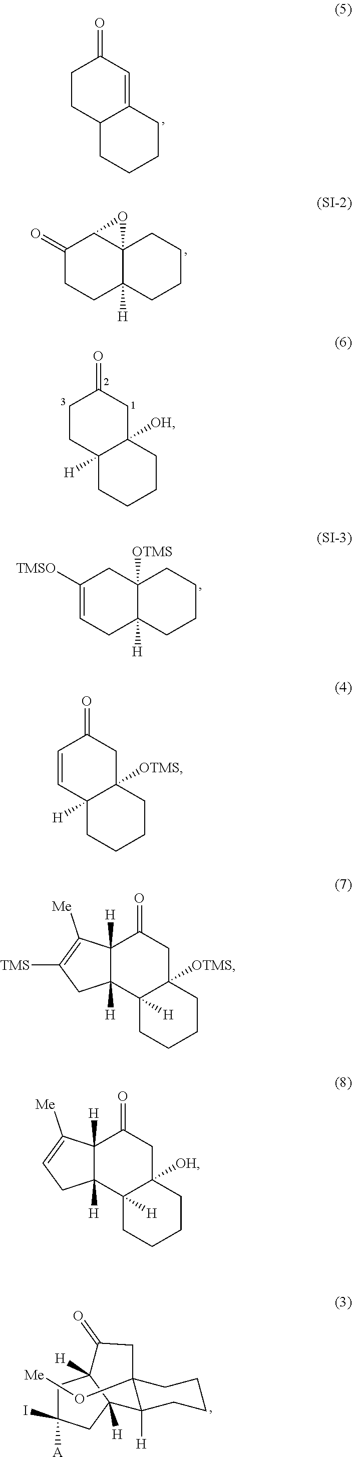

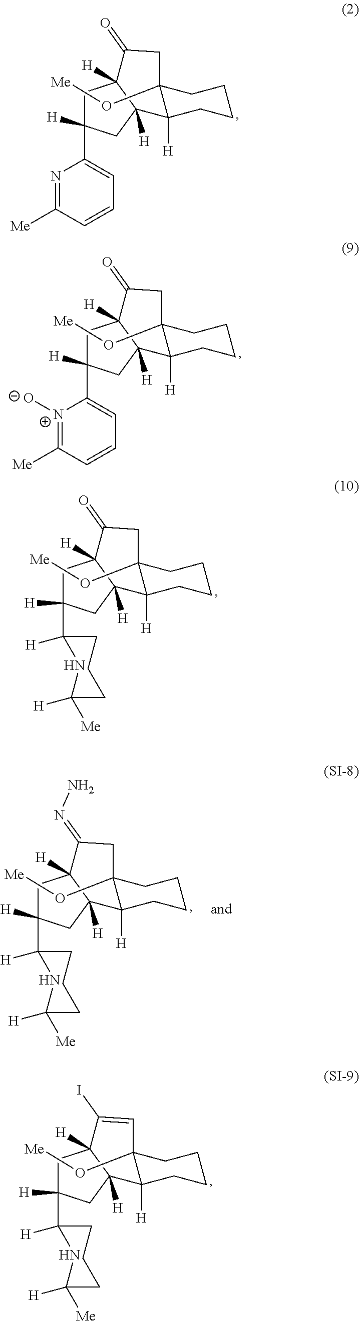

or an enantiomer, a scalemic mixture or a racemic mixture thereof, as an intermediate in the preparation of the scalemic or racemic mixture of Galbulimima alkaloid GB18.

### Embodiment 28

The process according to Embodiment 27, further comprising resolving the scalemic or racemic mixture of GB 18 into the enantiomers of Formulae (1) and (2).

### Embodiment 29

A compound selected from the group consisting of:

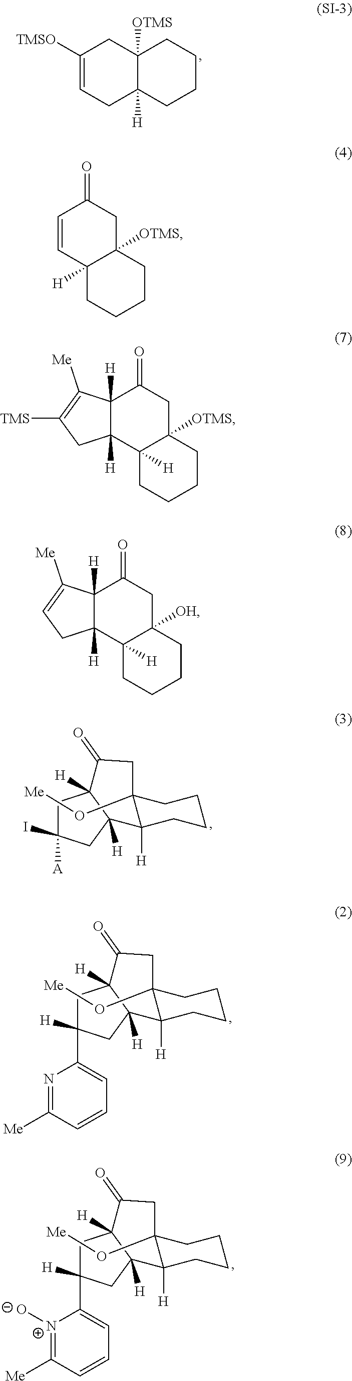

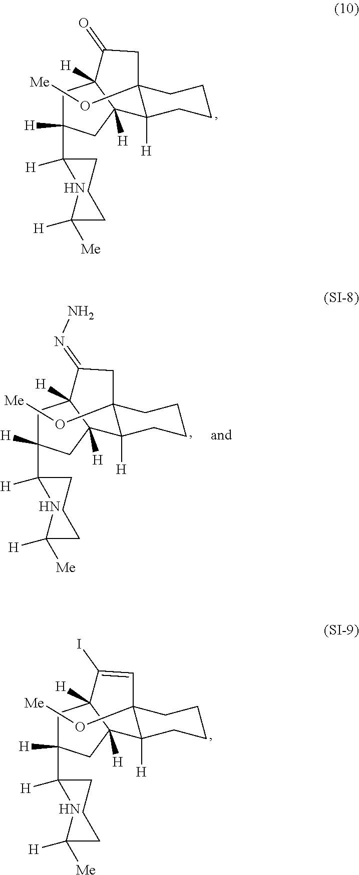

or an enantiomer, a scalemic mixture or a racemic mixture thereof.

### Embodiment 30

The compound of Formula (1)

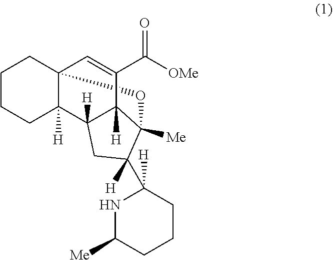 [SMILES: COC(=O)C1=C[C@]23CCCC[C@@H]2[C@H]2C[C@@H]([C@H]4CCC[C@@H](C)N4)[C@@](C)(O3)[C@@H]12]

or a pharmaceutically acceptable salt thereof.

### Embodiment 31

A method of antagonizing an opioid receptor in a subject in need of such antagonization, comprising administering to such subject a therapeutically effective amount of a compound of Formula (I)

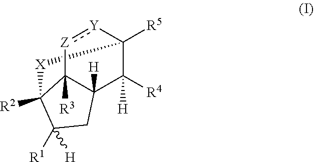

wherein:
[symbol] is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷;
   or R⁶ and R⁷ together form monocyclic or bicyclic C₃-C₁₄ cycloalkyl or C₃-C₁₄ heterocycloalkyl, each individually and optionally substituted with one or more R when [symbol] is a single bond;
   or R⁶ and R⁷ together form monocyclic or bicyclic C₅-C₁₄ heteroaryl optionally substituted with one or more R when [symbol] is a double bond;
Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
   each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇cycloalkyl), —NMsPh, and —C(═O)OH;
R² and R³ are independently H, C₁-C₆ alkyl, (C₁-C₆ alkyl)-C(═O)—, hydroxy, or —O—(C₁-C₆ alkyl);
   or R² and R³ together form C₃-C₇ cycloalkyl or C₃-C₇ heterocycloalkyl;
R⁴ and R⁵ are each independently H or C₁-C₆ alkyl;
   or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
   or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
   or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
   or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl); or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
each R^{p} independently is H, C₁-C₆ alkyl, —C(═O)—(C₁-C₆ alkyl), —Si(C₁-C₆ alkyl)₃, —S(═O)₂—(C₁-C₆ alkyl), —S(═O)₂—(C₆-C₁₀ aryl), or —NHTs;
R^{q}, R^{s}, and R^{t} are each independently H or C₁-C₆ alkyl;
   wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
   wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
   or a pharmaceutically acceptable salt thereof.

### Embodiment 32

The method according to embodiment 31, wherein, in Formula (I):
[symbol] is a double bond;
X is O;
Y is CR⁶;
Z is CR⁷;
R¹ is C₆-C₁₀ aryl or C₅-C₁₀ heteroaryl, optionally substituted with 1-3 substituents independently selected from the group consisting of C₁-C₆ alkyl, halo, —OH, —O—C₁-C₆ alkyl, —CN, —NO₂, —CF₃, methylene cyclopropyl, and —C(═O)OH;
R² is C₁-C₆ alkyl;
R³ is H;
R⁴ and R⁵ together with the carbon atoms to which they are shown attached form a C₃-C₇ cycloalkyl, which is optionally substituted with 1-2 substitutents independently selected from the group consisting of C₁-C₆ alkyl, halo, —OH, —O—C₁-C₆ alkyl, —CN, —NO₂, —CF₃, and —C(═O)OH;
R⁶ is H; and
R⁷ is —C(═O)—(C₁-C₆ alkyl).

### Embodiment 33

The method according to embodiment 31 or 32, wherein the compound of Formula (I) or a pharmaceutically acceptable salt thereof is the compound of Formula (2):

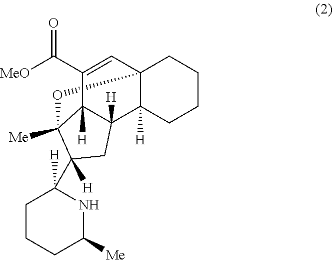 [SMILES: COC(=O)C1=C[C@@]23CCCC[C@H]2[C@@H]2C[C@H]([C@@H]4CCC[C@H](C)N4)[C@](C)(O3)[C@H]12]

or a pharmaceutically acceptable salt thereof.

### Embodiment 34a

The method according to embodiment 31, wherein the compound of Formula (I) or a pharmaceutically acceptable salt thereof is selected from the group consisting of:

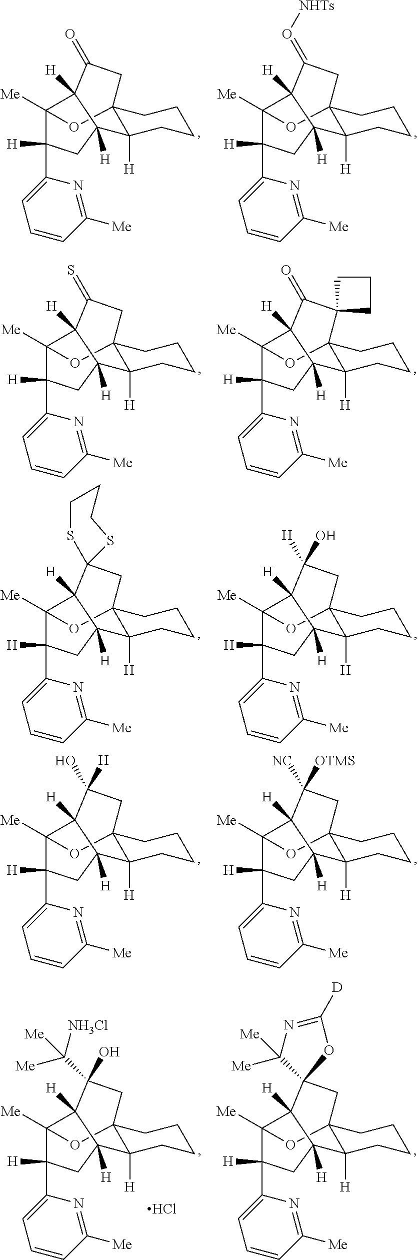

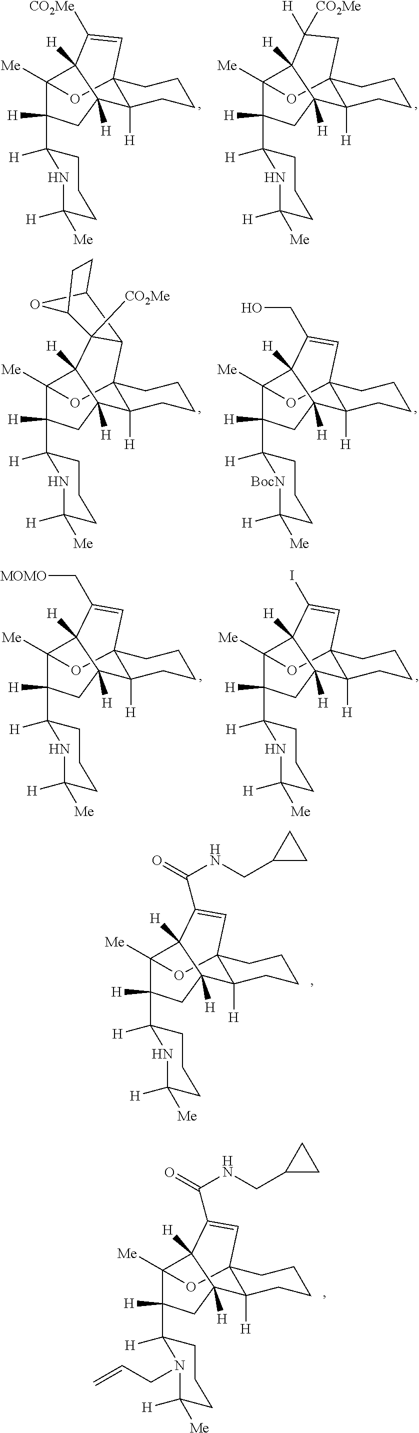

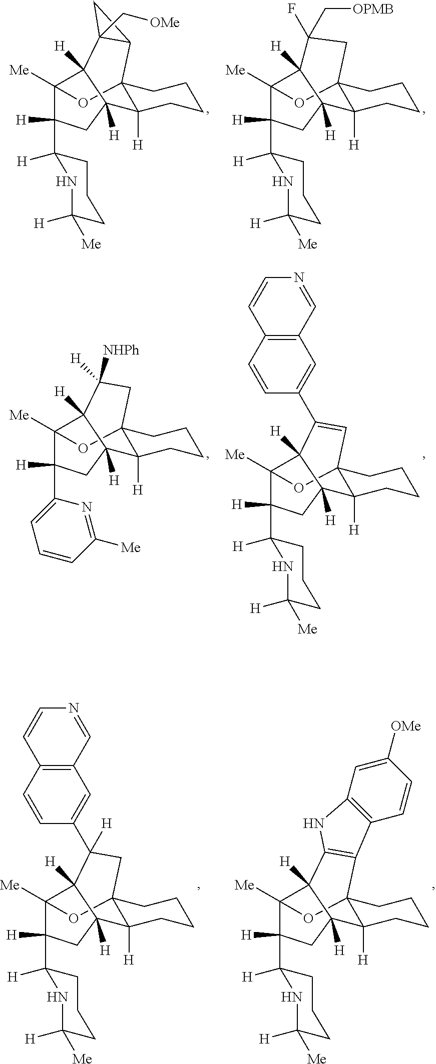

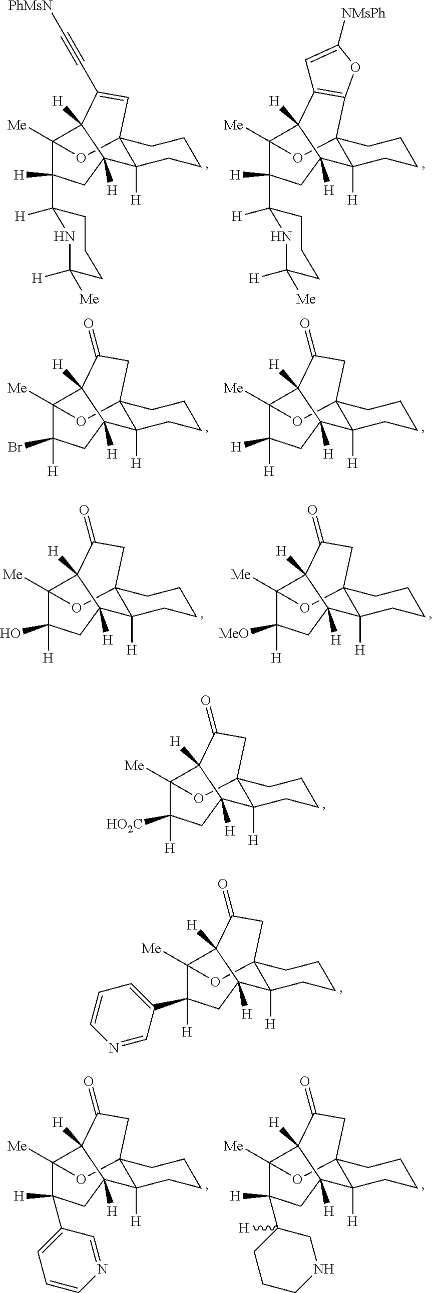

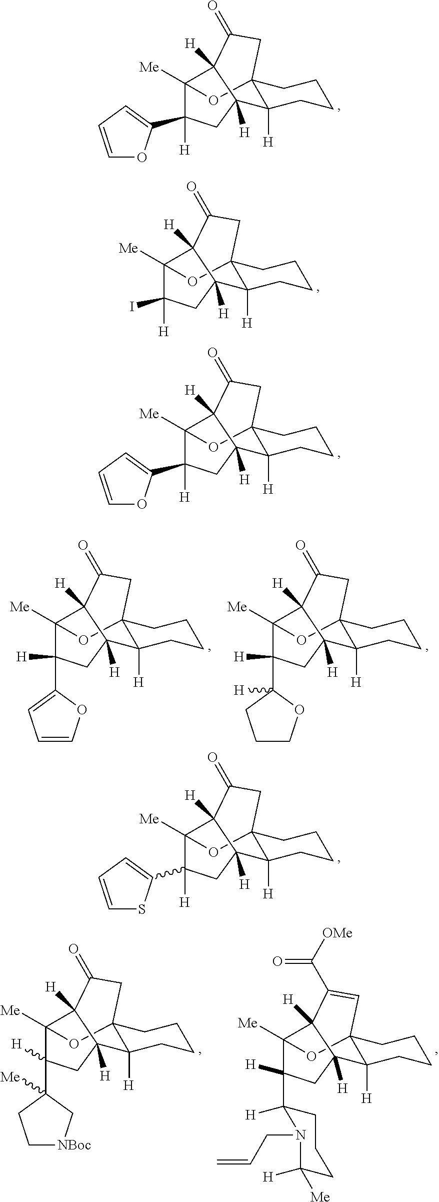

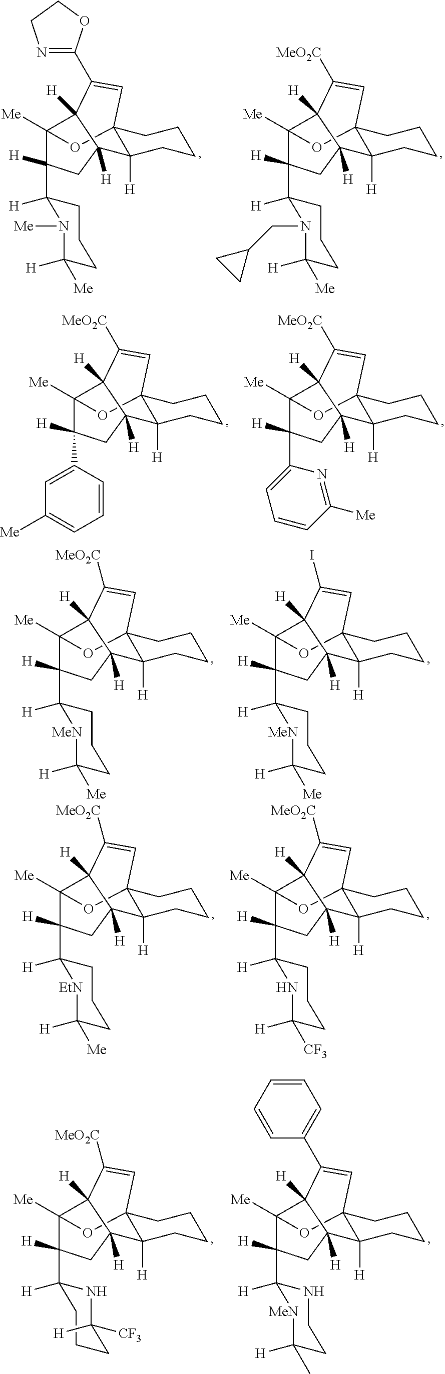

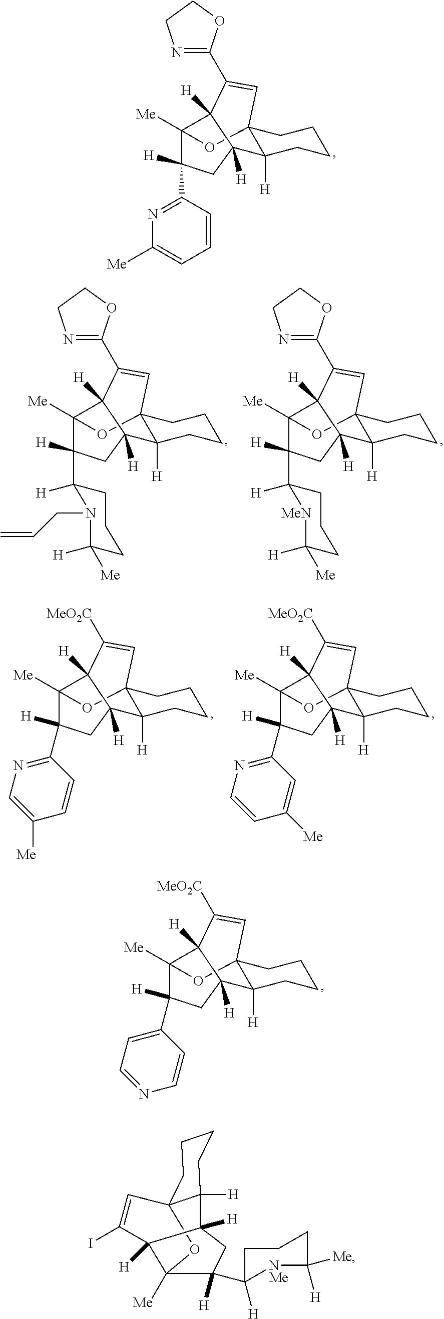

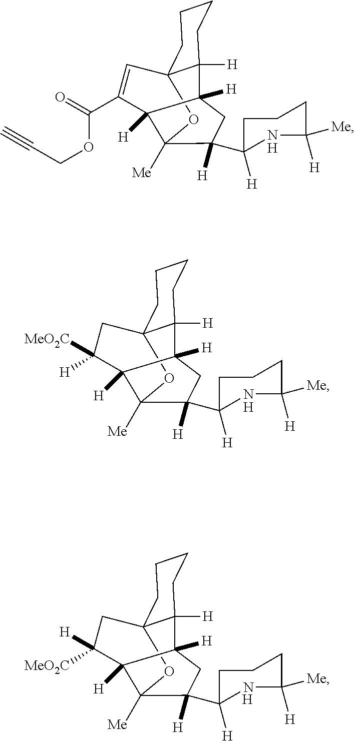

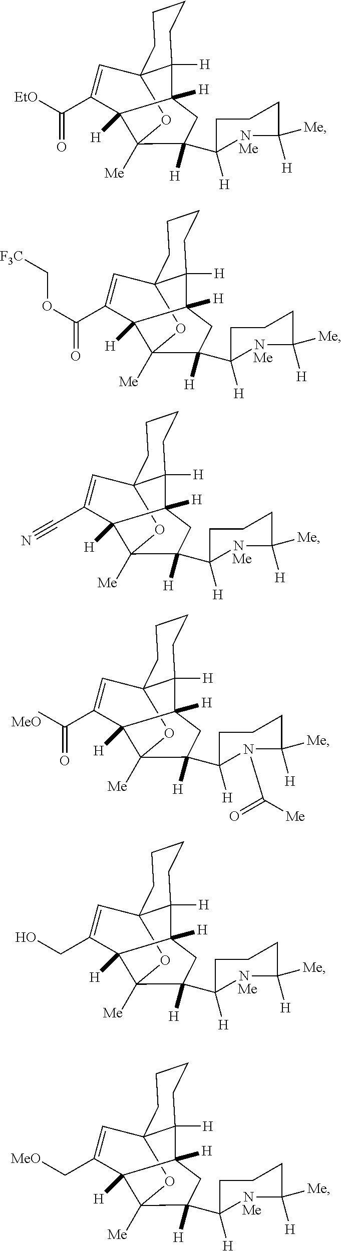

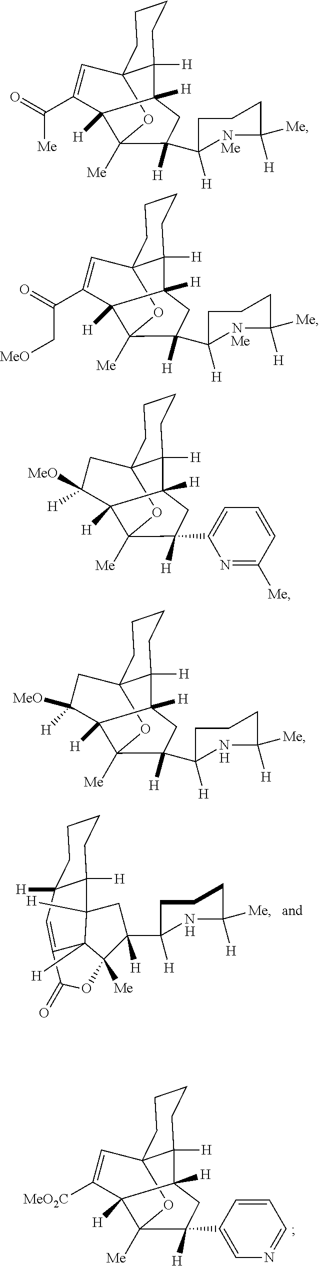

or a pharmaceutically acceptable salt thereof.

### Embodiment 34b

The method according to embodiment 31, wherein the compound of Formula (I) or a pharmaceutically acceptable salt thereof is selected from the group consisting of:

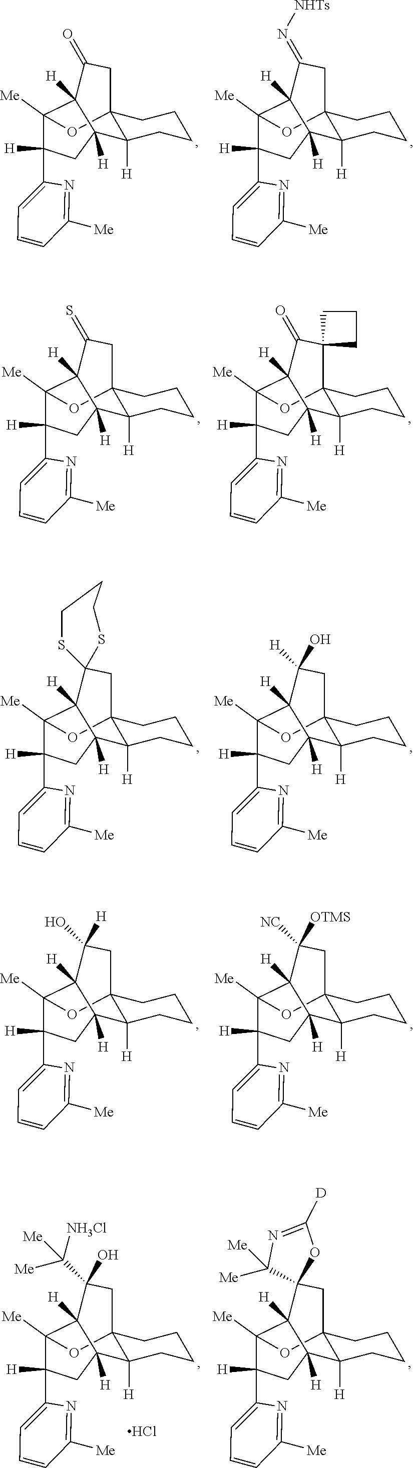

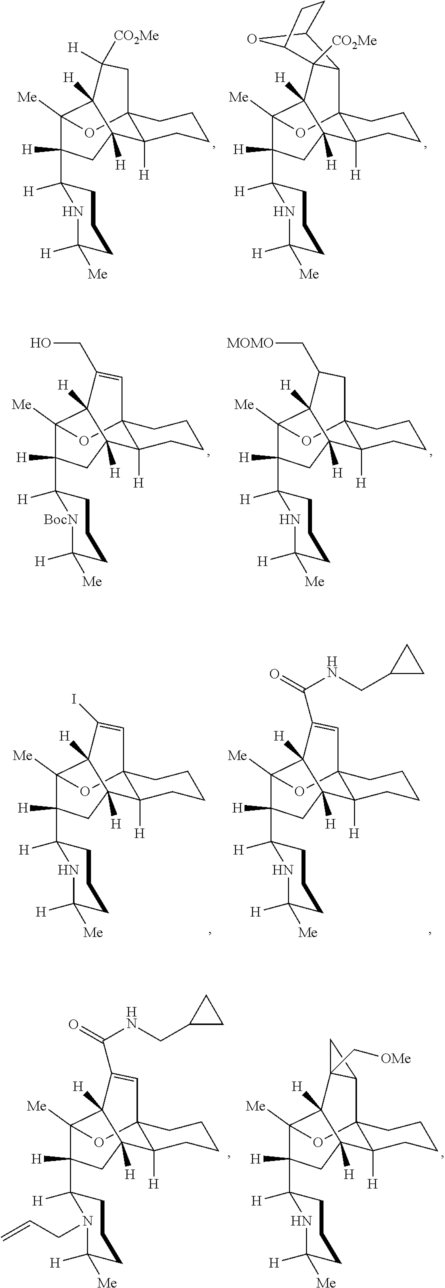

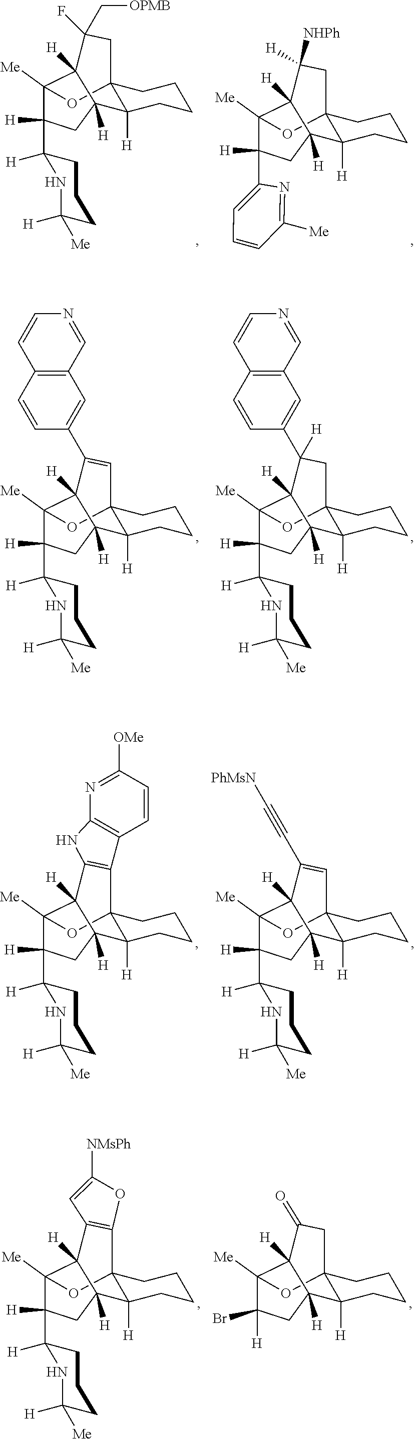

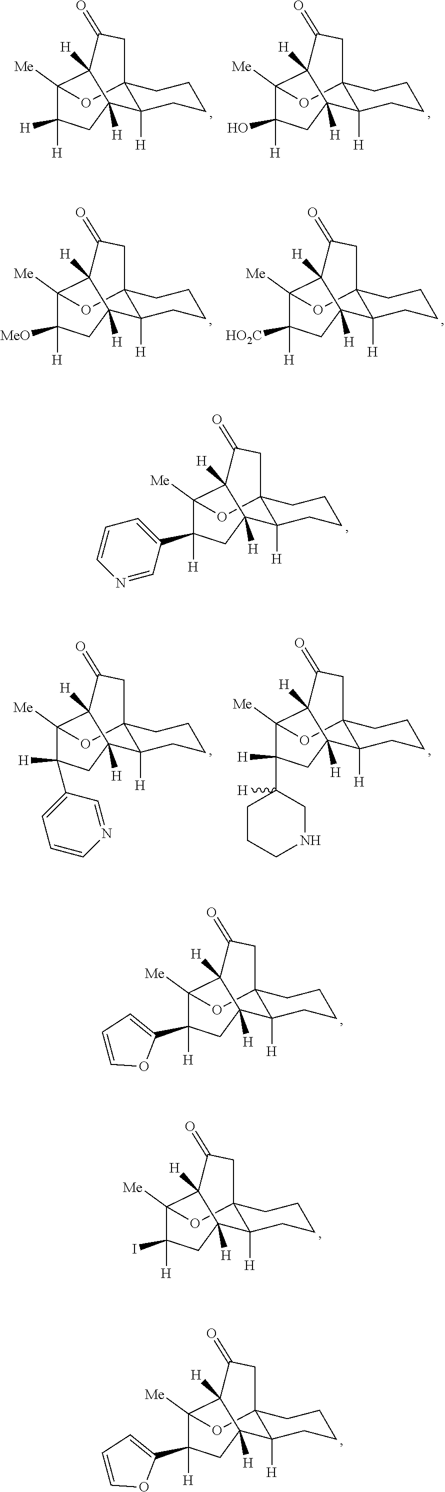

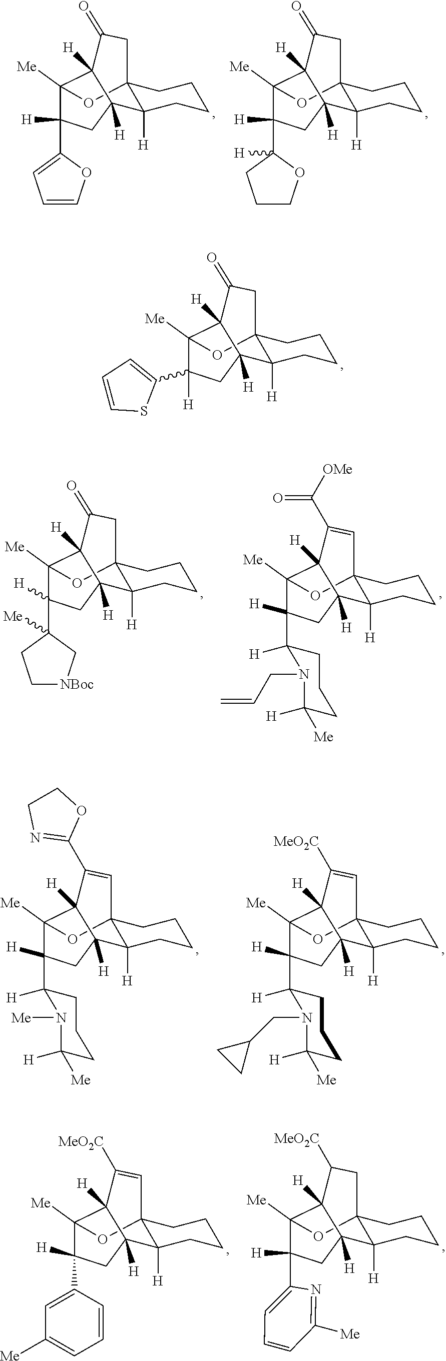

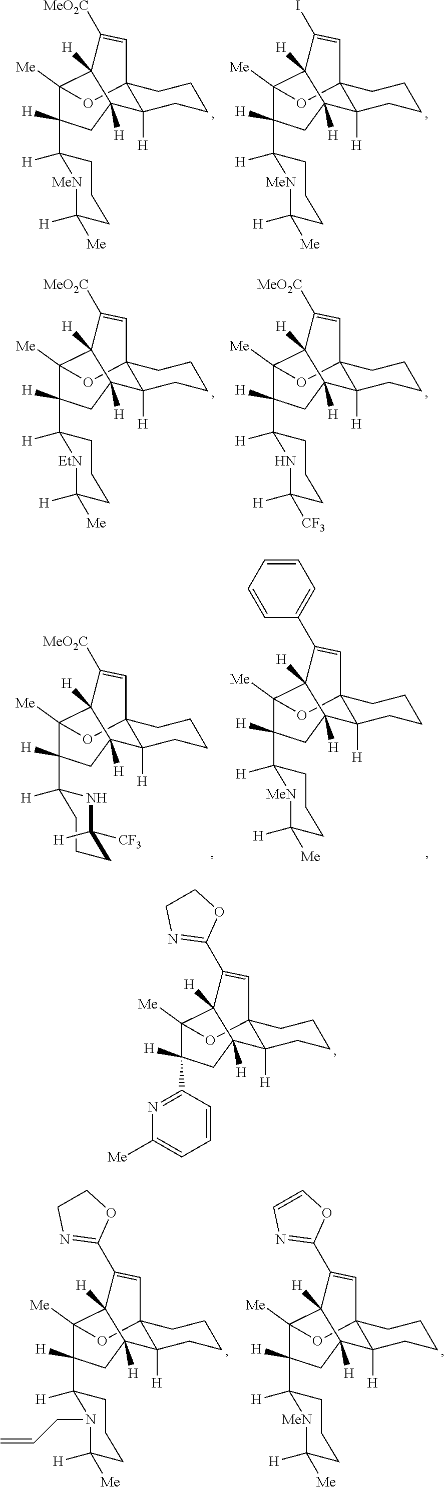

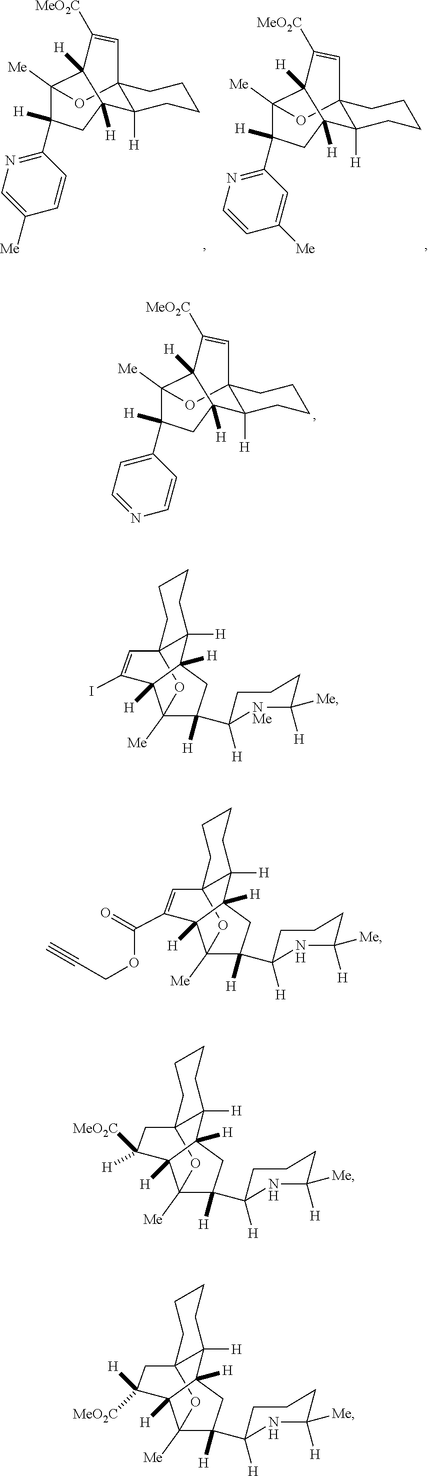

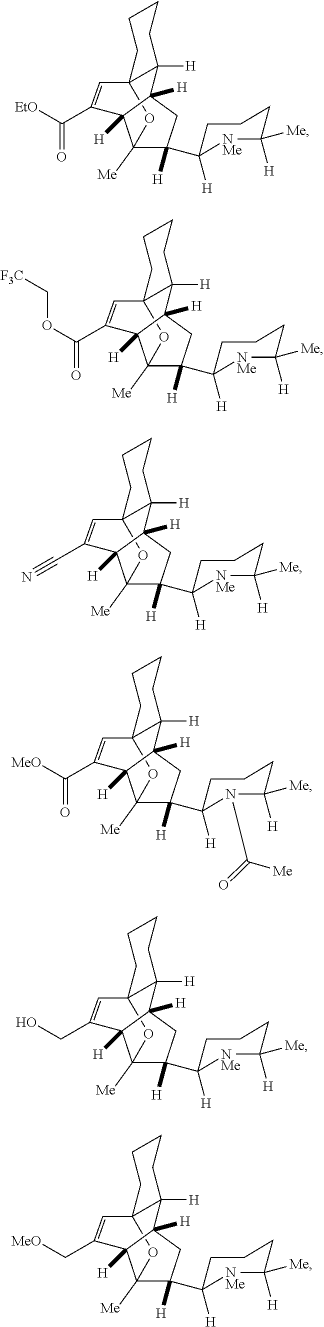

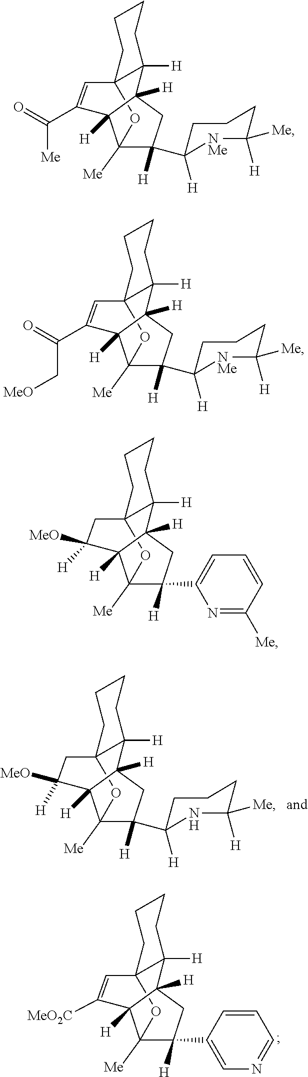

or a pharmaceutically acceptable salt thereof.

Exemplary schemes (and partial schemes) illustrating the preparation of such compounds are shown below:

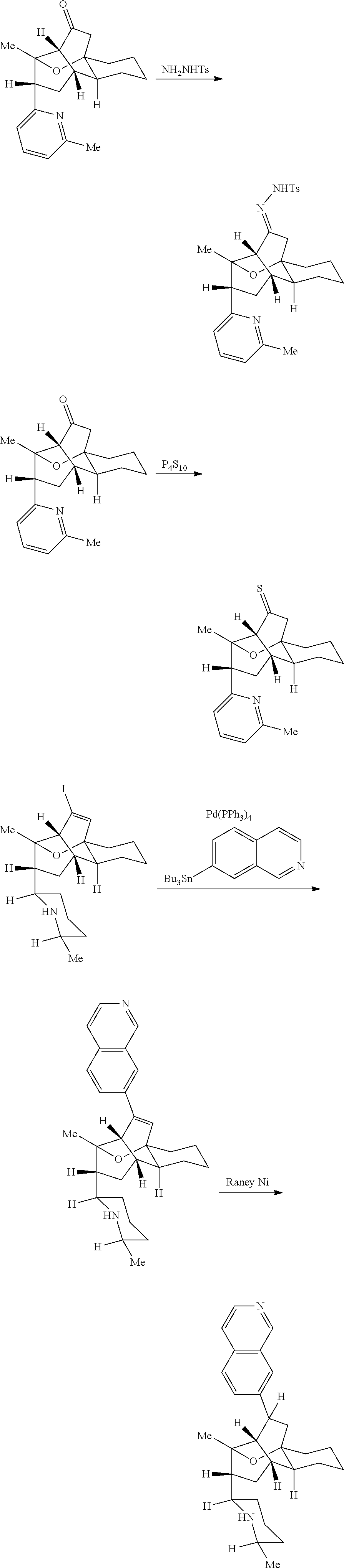

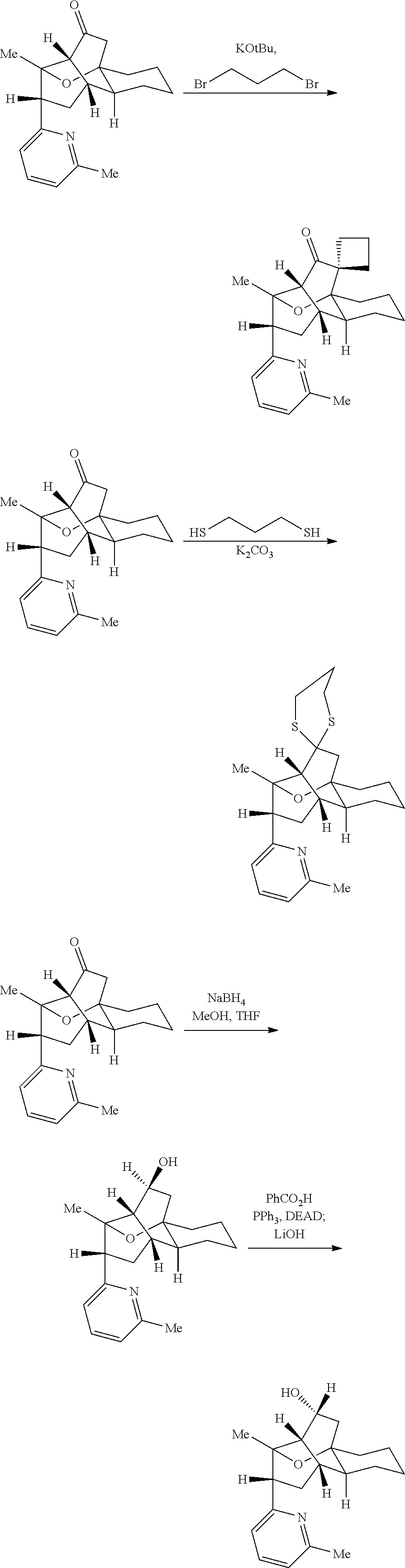

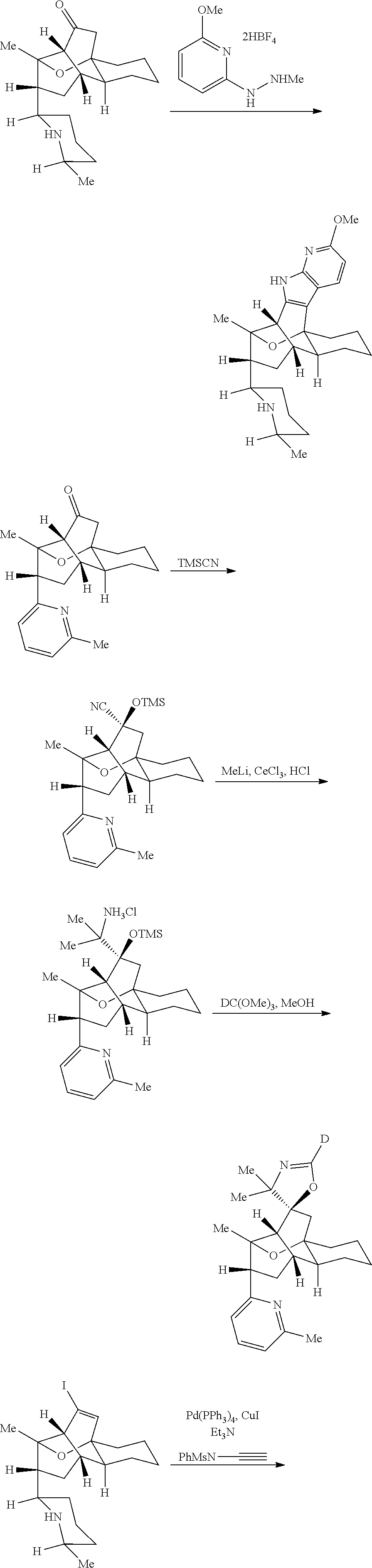

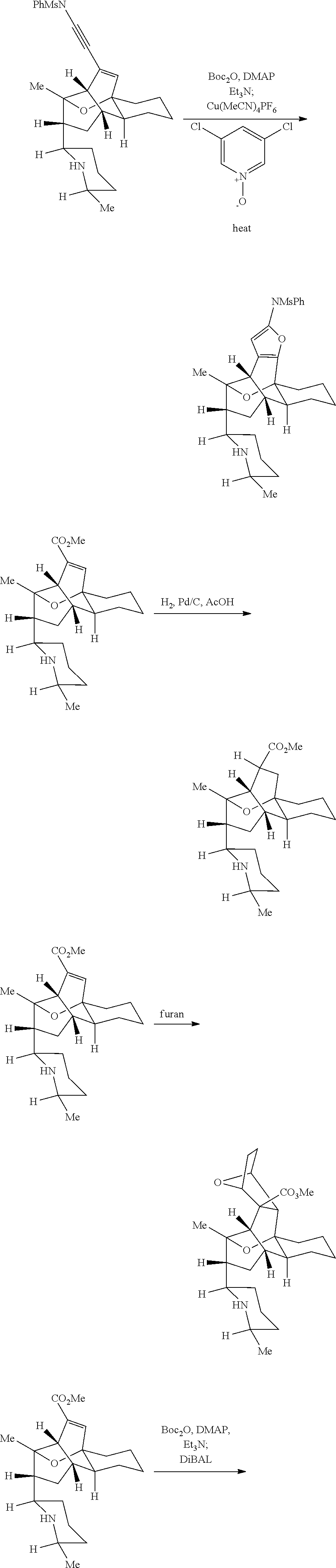

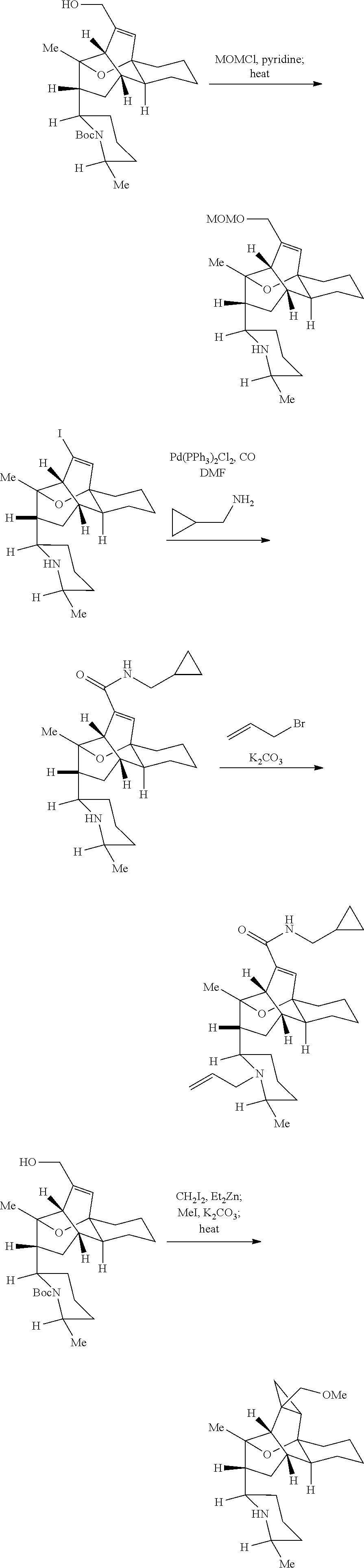

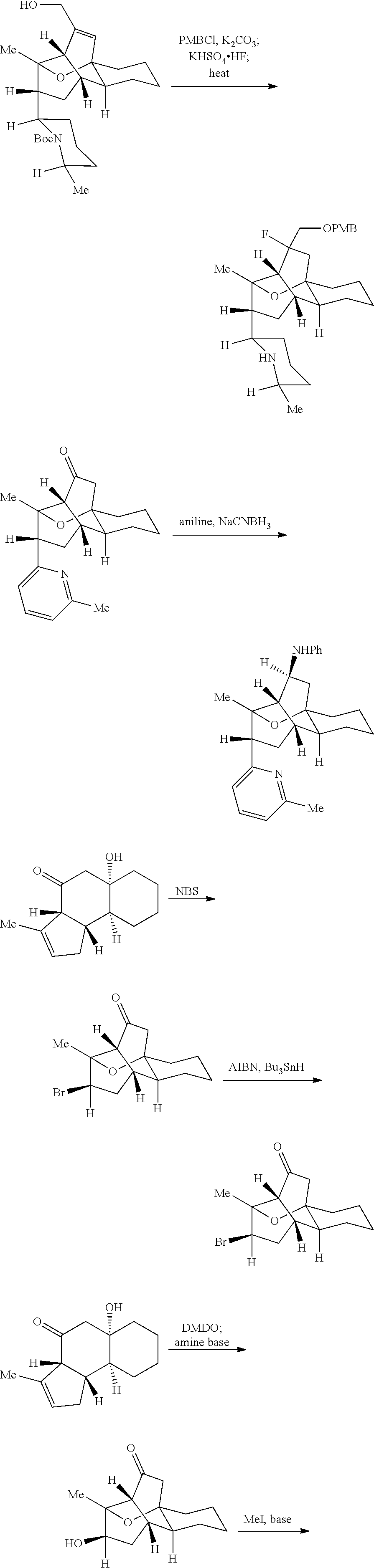

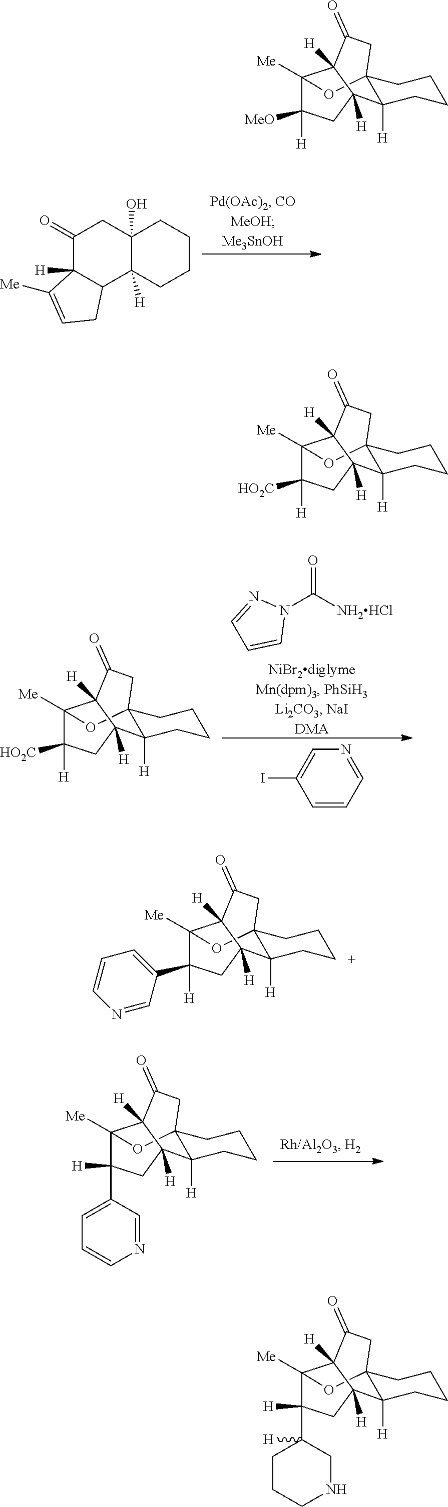

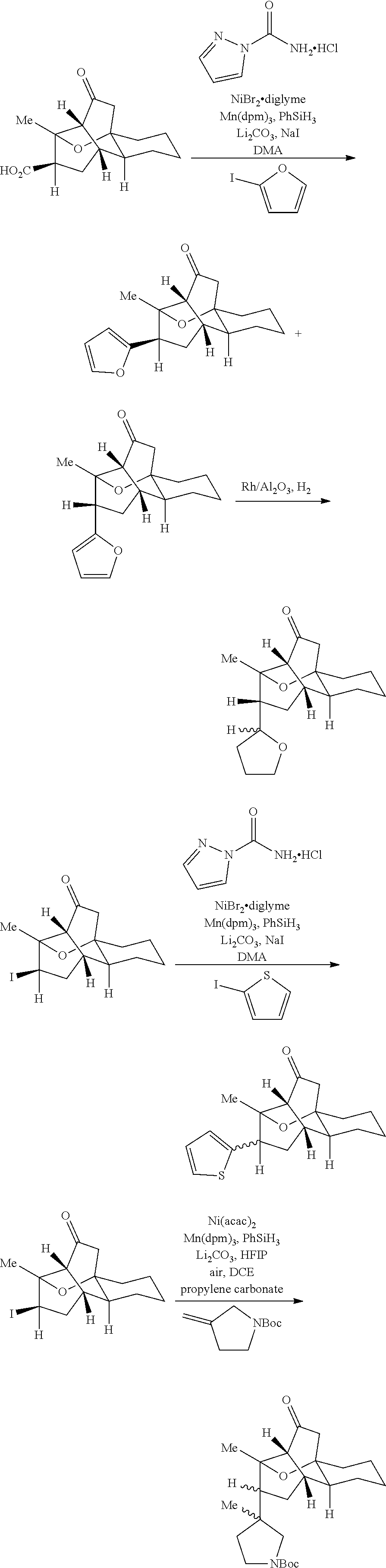

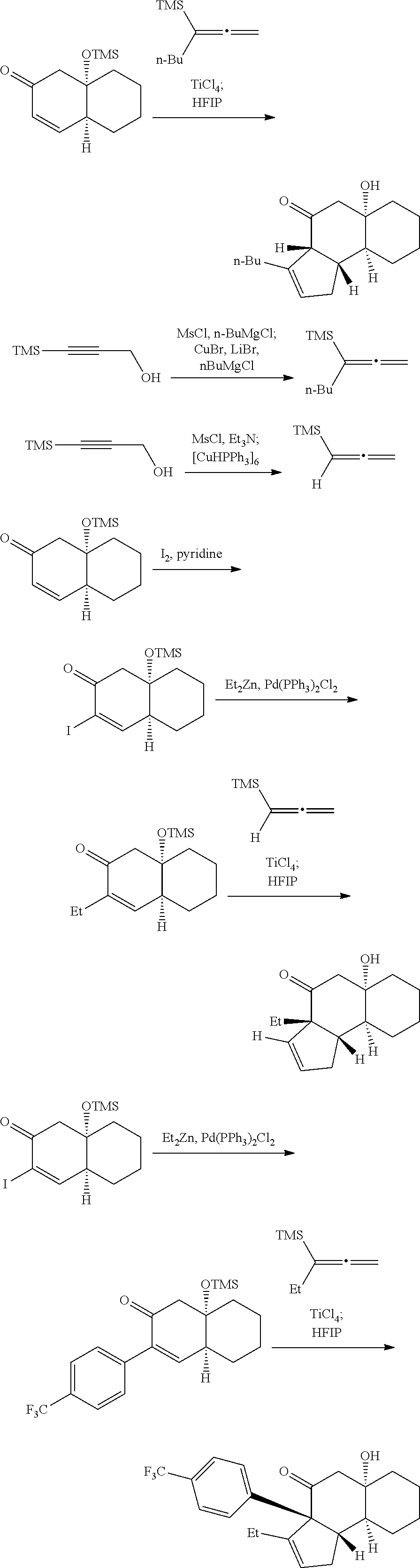

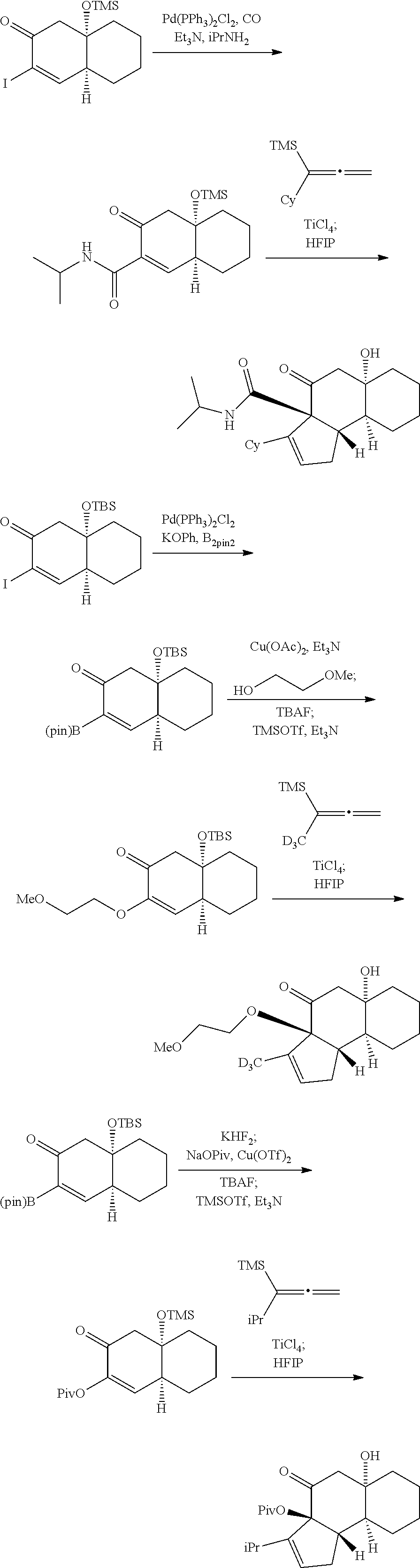

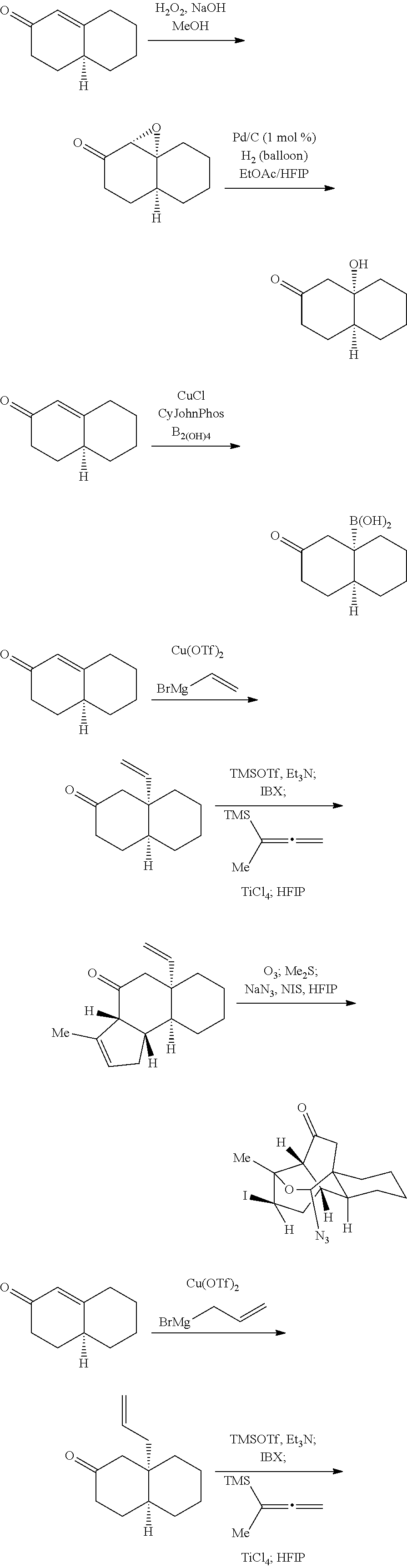

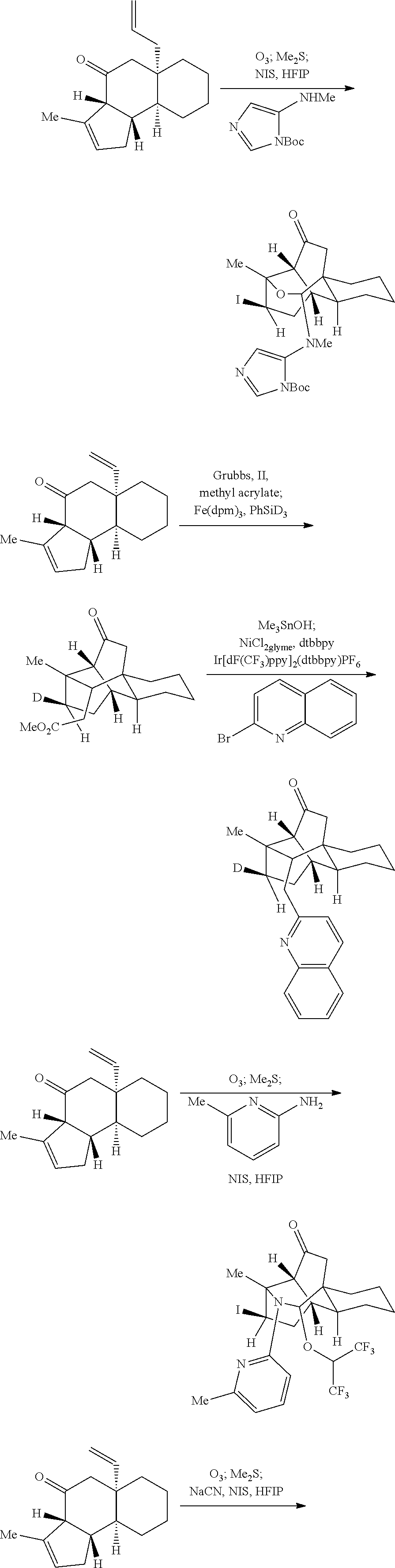

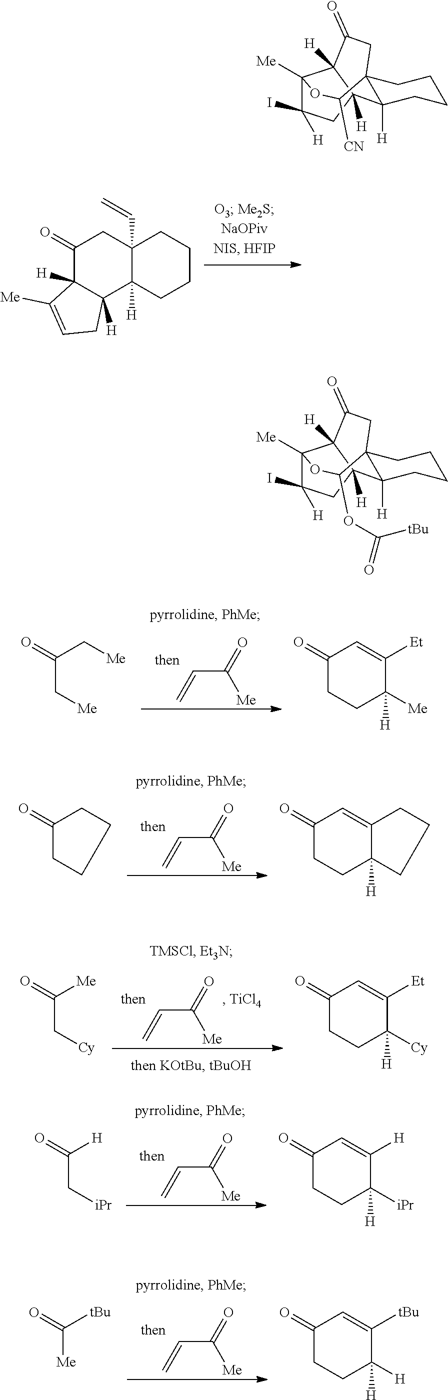

### Embodiment 35

The method according to any one of embodiments 31-33, wherein the opioid receptor is a mu-opioid receptor (MOR), a kappa-opioid receptor (KOR) or a delta-opioid receptor (DOR).

### Embodiment 36

The method according to embodiment 35, wherein the opioid receptor is a KOR or MOR.

### Embodiment 37

A method of treating a disorder selected from the group consisting of substance abuse disorder, major depressive disorder, resistant depression, and impulse control disorder in a subject in need of such treatment comprising administering to the subject a therapeutically effective amount of a compound of Formula (I)

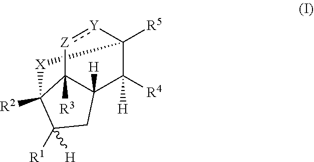

wherein:
[symbol] is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷; Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
   each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇cycloalkyl), —NMsPh, and —C(═O)OH;
R² and R³ are independently H, C₁-C₆ alkyl, (C₁-C₆ alkyl)-C(═O)—, hydroxy, or —O—(C₁-C₆ alkyl);
   or R² and R³ together form C₃-C₇ cycloalkyl or C₃-C₇ heterocycloalkyl;
R⁴ and R⁵ are each independently H or C₁-C₆ alkyl;
   or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
   or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
   or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
   or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
   or R⁶ and R⁷ together form monocyclic or bicyclic C₃-C₁₄ cycloalkyl or C₃-C₁₄ heterocycloalkyl, each individually and optionally substituted with 1-3 R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl);
   or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
   each R^{p} independently is H, C₁-C₆ alkyl, (C₁-C₆ alkyl)-C(═O)—, —Si(C₁-C₆ alkyl)₃, —S(═O)₂—(C₁-C₆ alkyl), or —S(═O)₂—(C₆-C₁₀ aryl);
   R^{q}, R^{s}, and R^{t} are each independently H or C₁-C₆ alkyl;
   wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
   wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
   or a pharmaceutically acceptable salt thereof.

### Embodiment 38

The method according to embodiment 37, wherein, in Formula (I):
[symbol] is a double bond;
X is O;
Y is CR⁶;
Z is CR⁷;
R¹ is C₆-C₁₀ aryl or C₅-C₁₀ heteroaryl, optionally substituted with 1-3 substituents independently selected from the group consisting of C₁-C₆ alkyl, halo, —OH, —O—C₁-C₆ alkyl, —CN, —NO₂, —CF₃, methylene cyclopropyl, and —C(═O)OH;
R² is C₁-C₆ alkyl;
R³ is H;
R⁴ and R⁵ together with the carbon atoms to which they are shown attached form a C₃-C₇ cycloalkyl, which is optionally substituted with 1-2 substitutents independently selected from the group consisting of C₁-C₆ alkyl, halo, —OH, —O—C₁-C₆ alkyl, —CN, —NO₂, —CF₃, and —C(═O)OH;
R⁶ is H; and
R⁷ is —C(═O)—(C₁-C₆ alkyl).

### Embodiment 39

The method according to embodiment 37 or 38, wherein the compound of Formula (I) or a pharmaceutically acceptable salt thereof is the compound of Formula (2):

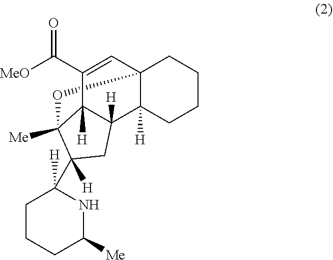 [SMILES: COC(=O)C1=C[C@@]23CCCC[C@H]2[C@@H]2C[C@H]([C@@H]4CCC[C@H](C)N4)[C@](C)(O3)[C@H]12]

or a pharmaceutically acceptable salt thereof.

### Embodiment 40a

The method according to embodiment 37, wherein the compound of Formula (I) or a pharmaceutically acceptable salt thereof is selected from the group consisting of:

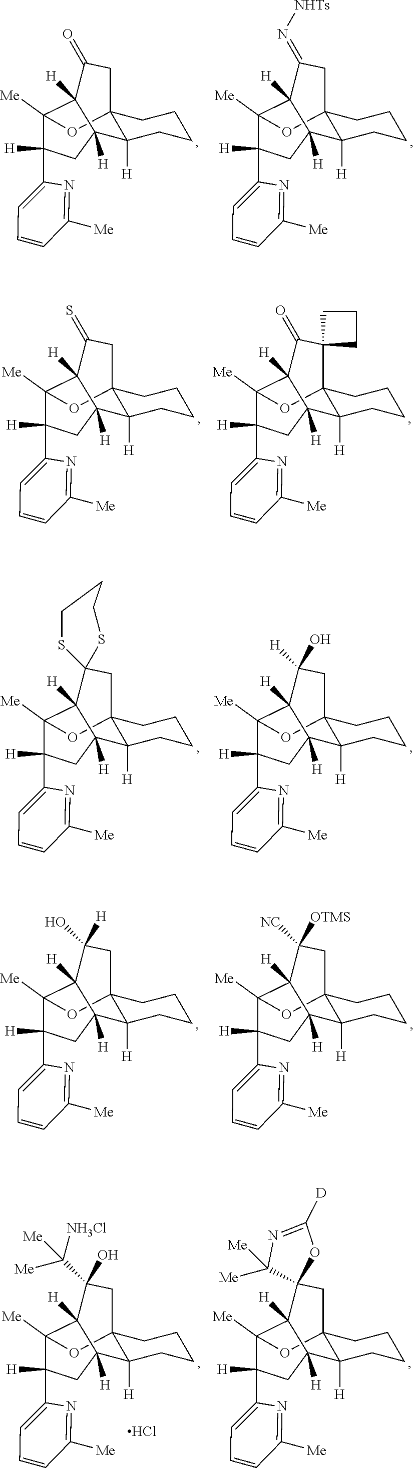

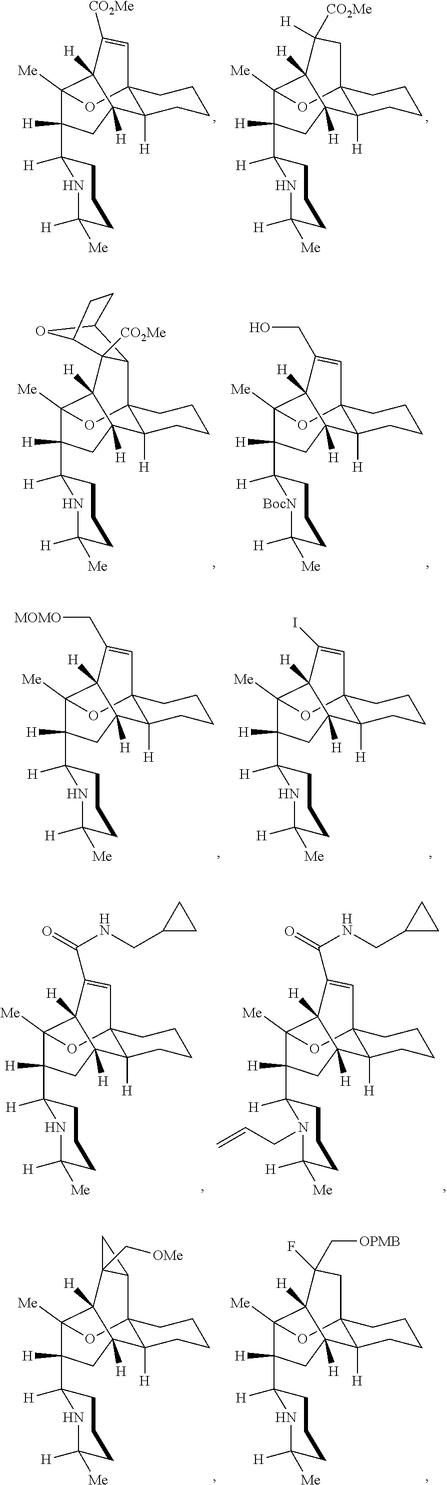

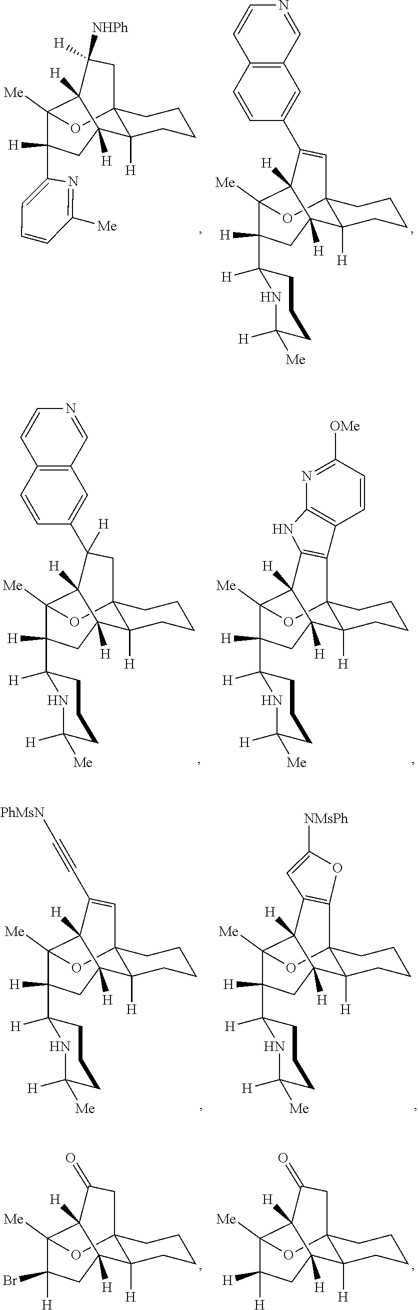

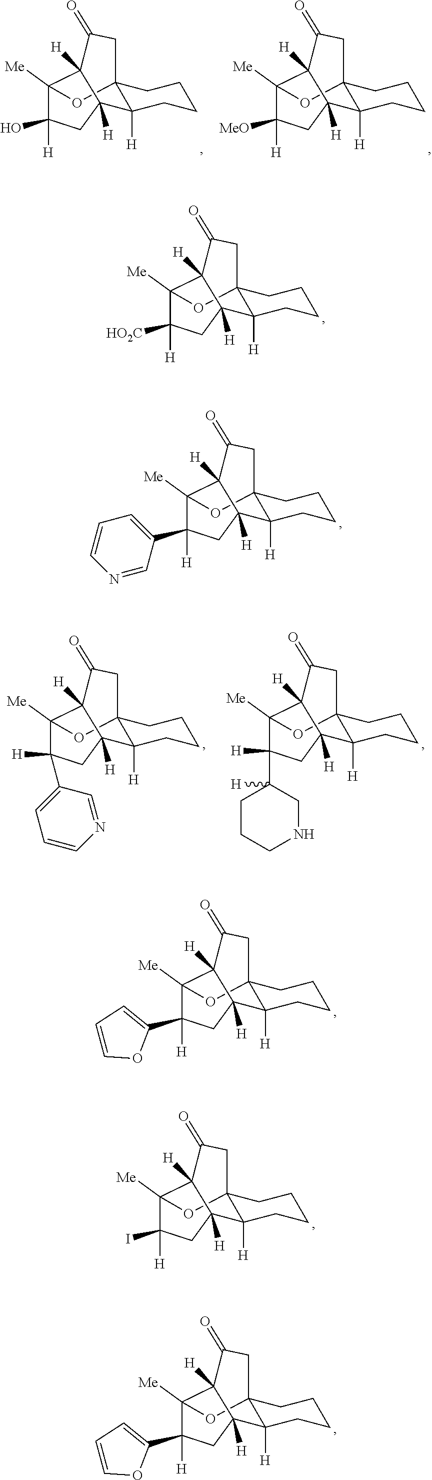

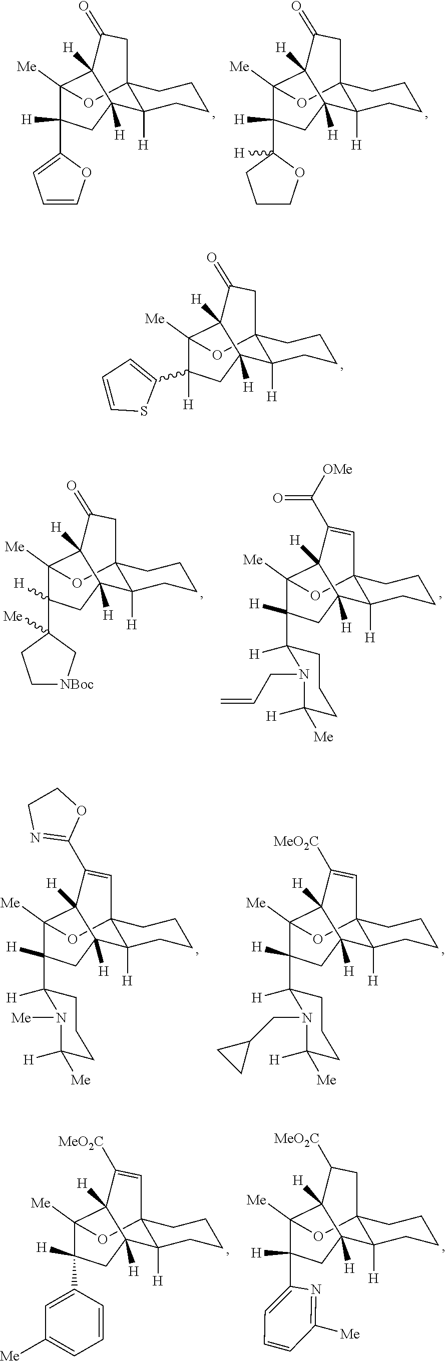

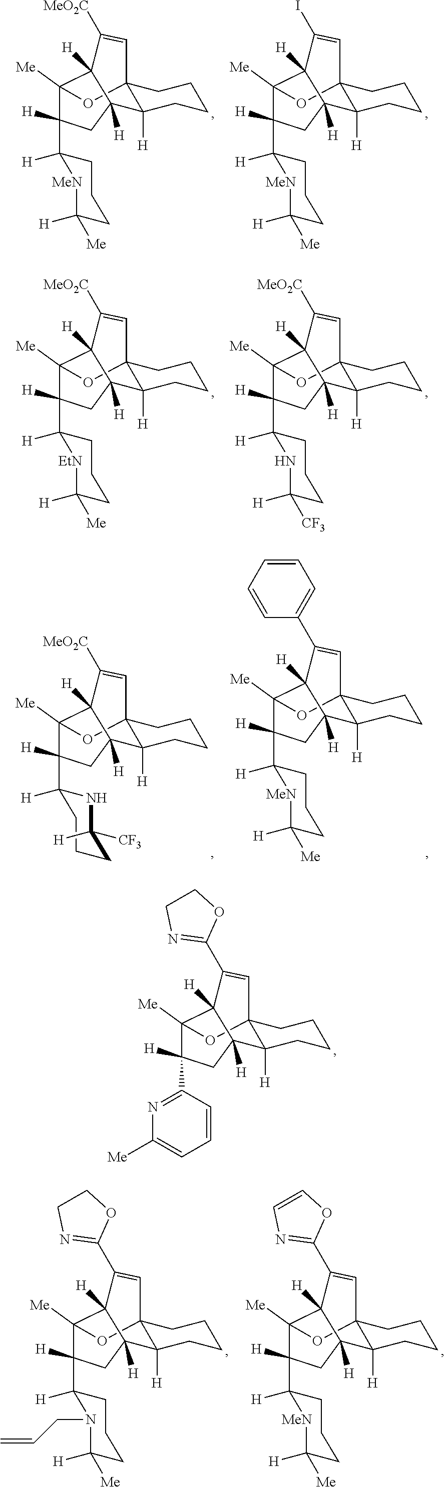

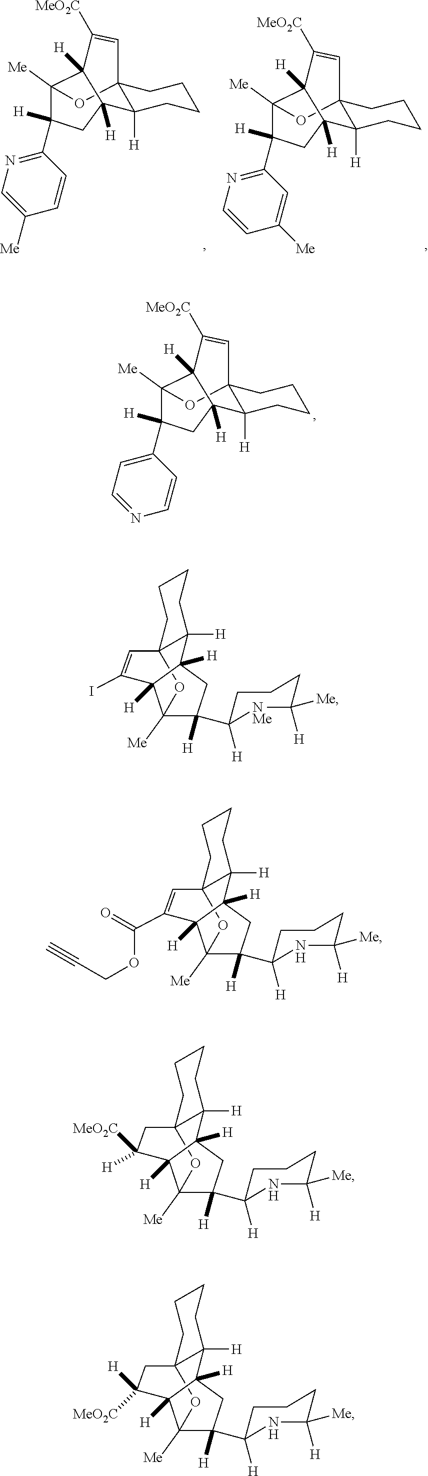

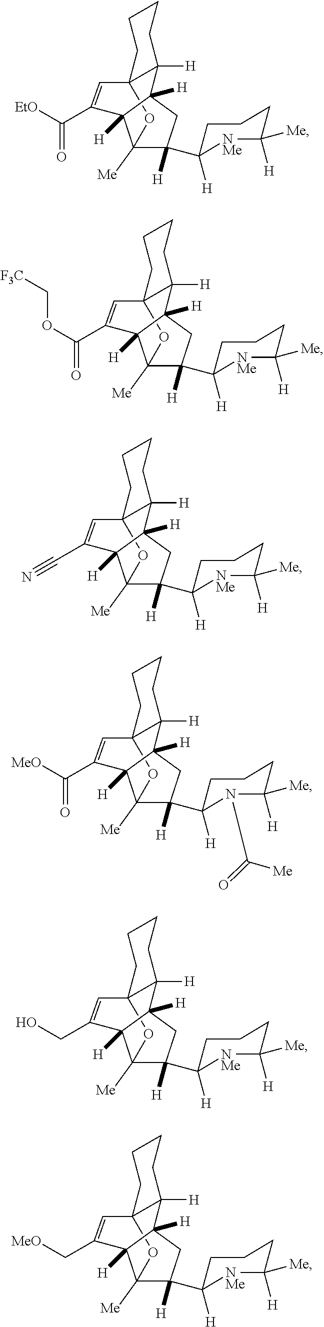

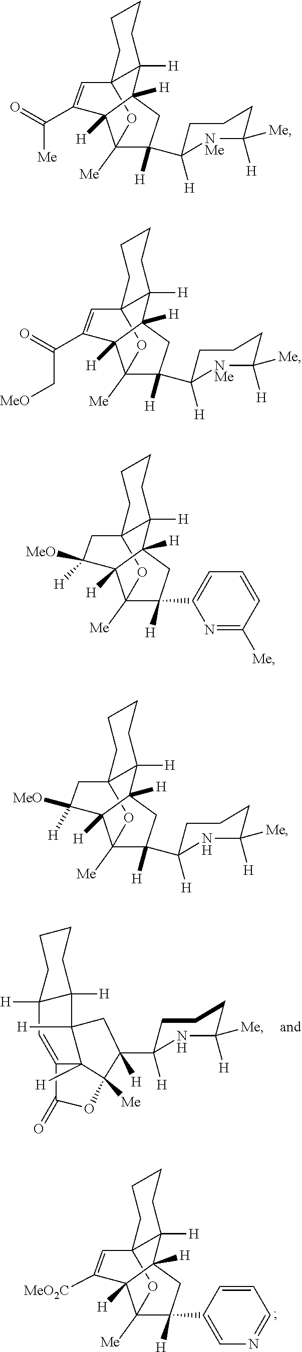

or a pharmaceutically acceptable salt thereof.

### Embodiment 40b

The method according to embodiment 37, wherein the compound of Formula (I) or a pharmaceutically acceptable salt thereof is selected from the group consisting of:

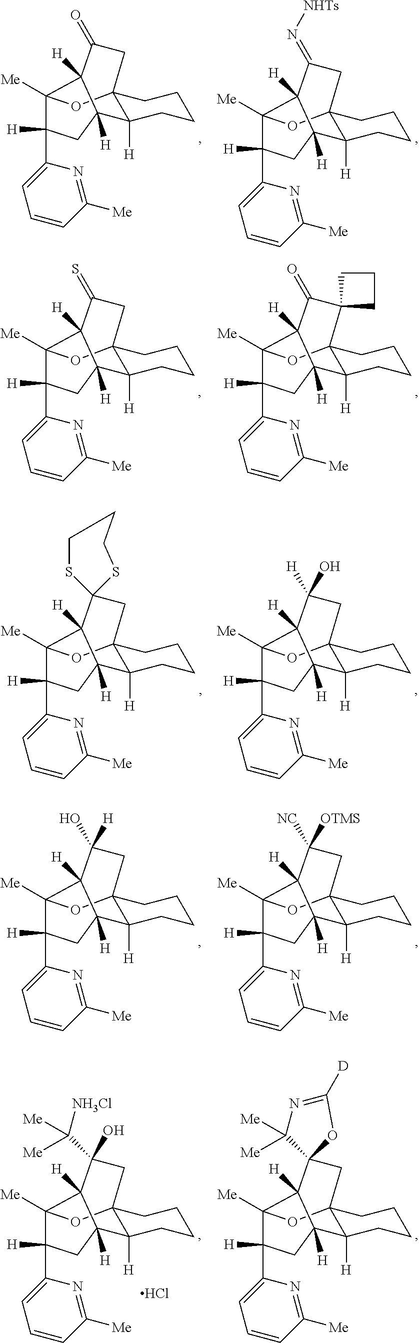

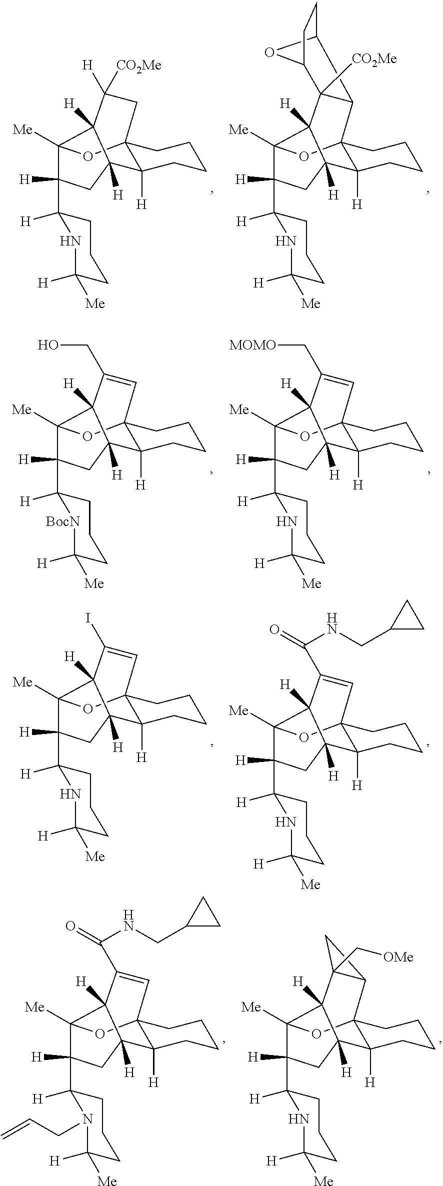

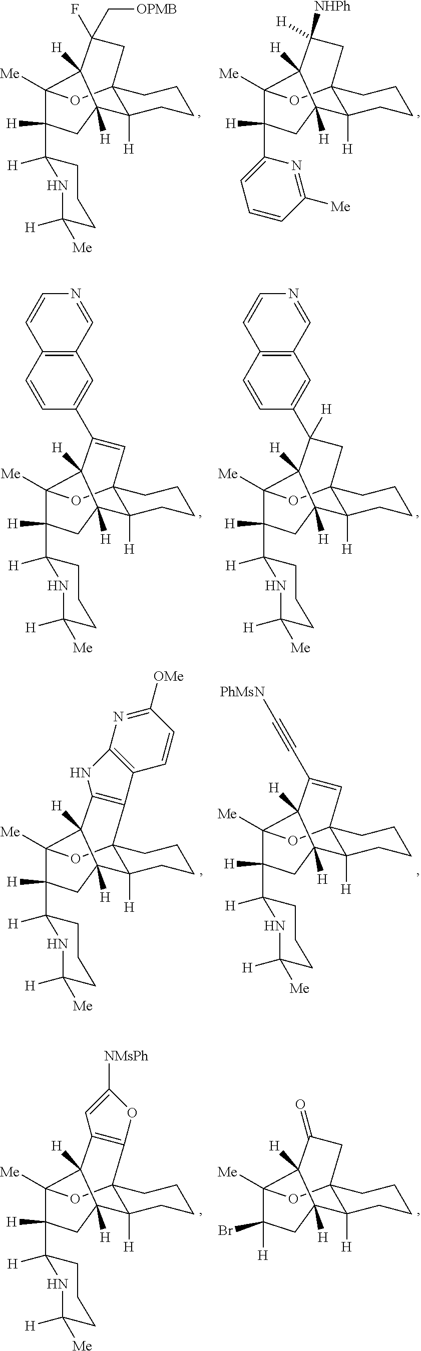

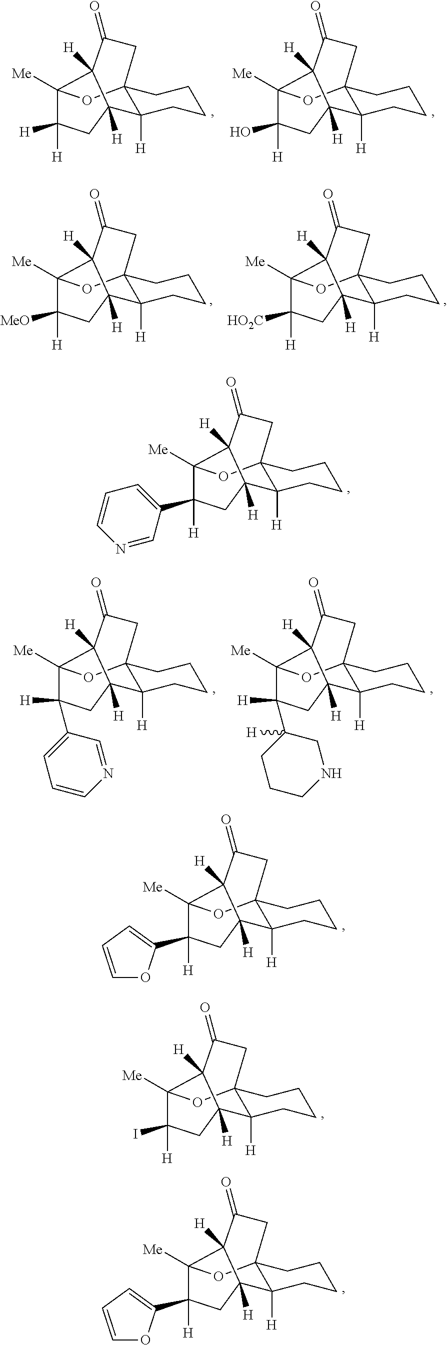

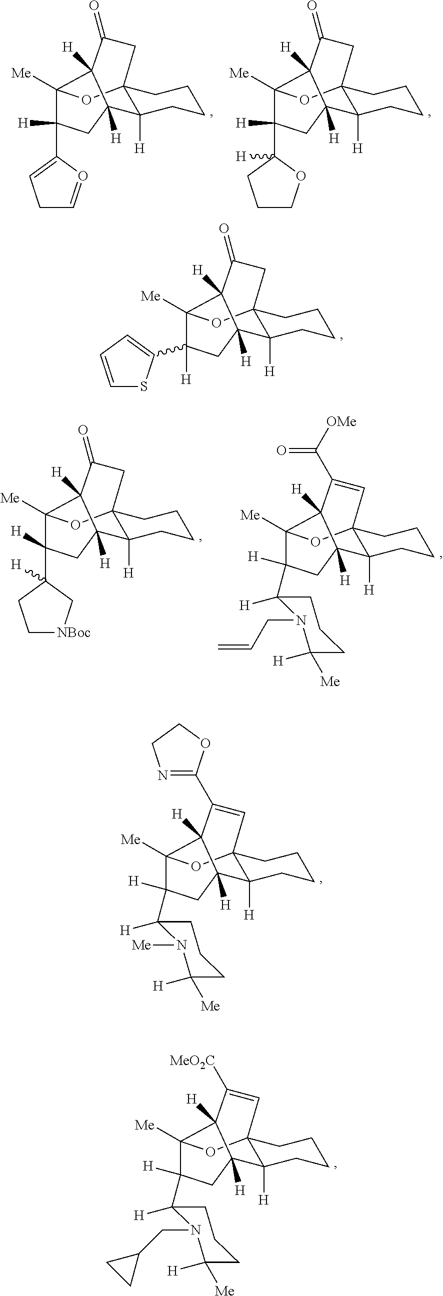

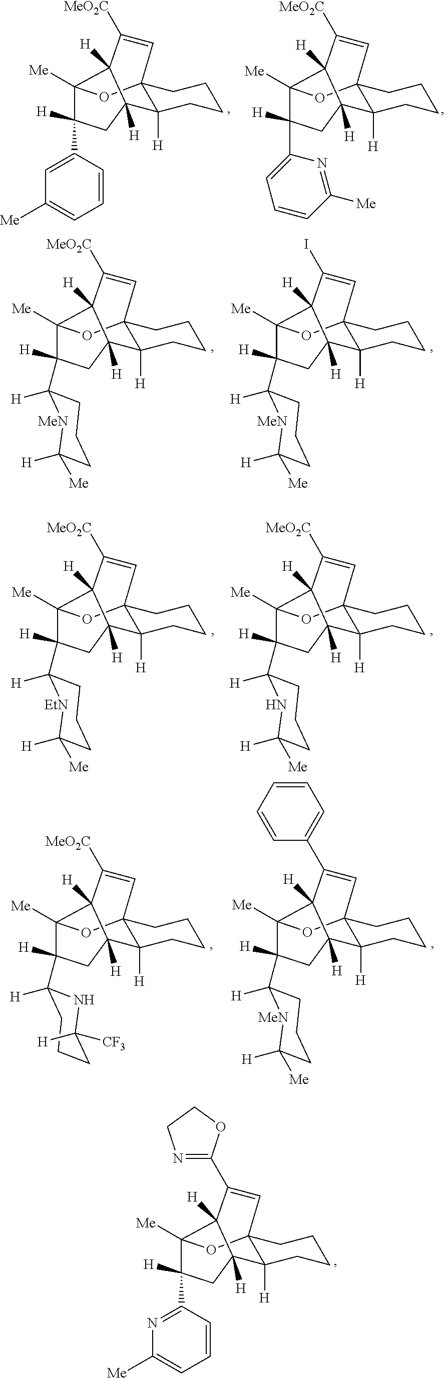

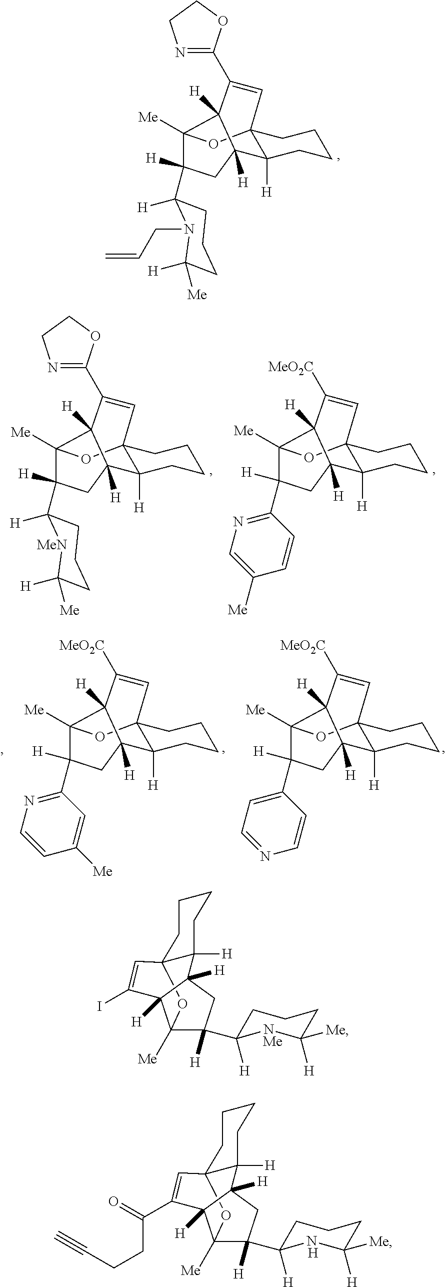

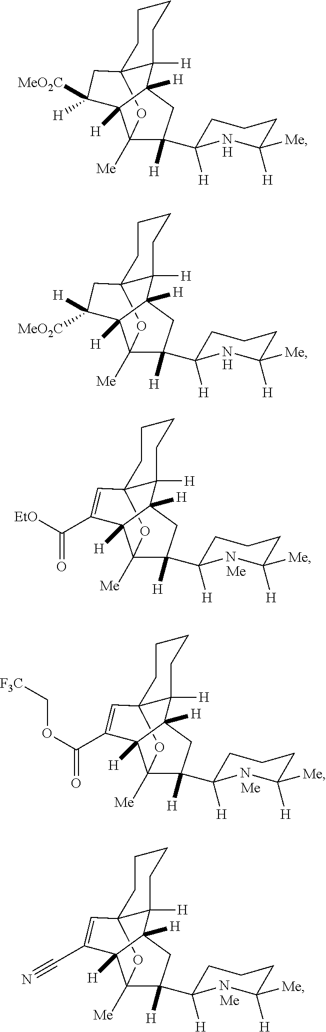

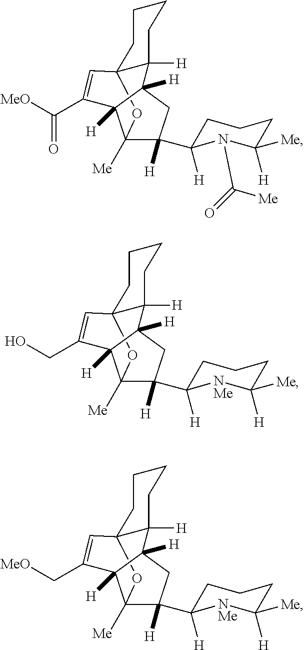 [SMILES: COC(=O)C1=CC23CCCCC2[C@@H]2C[C@H](C4CCCC(C)N4C(C)=O)C(C)(O3)[C@H]12 ; CC1CCCC([C@H]2C[C@H]3C4CCCCC45C=C(CO)[C@H]3C2(C)O5)N1 ; COCC1=CC23CCCCC2[C@@H]2C[C@H](C4CCCC(C)N4)C(C)(O3)[C@H]12]

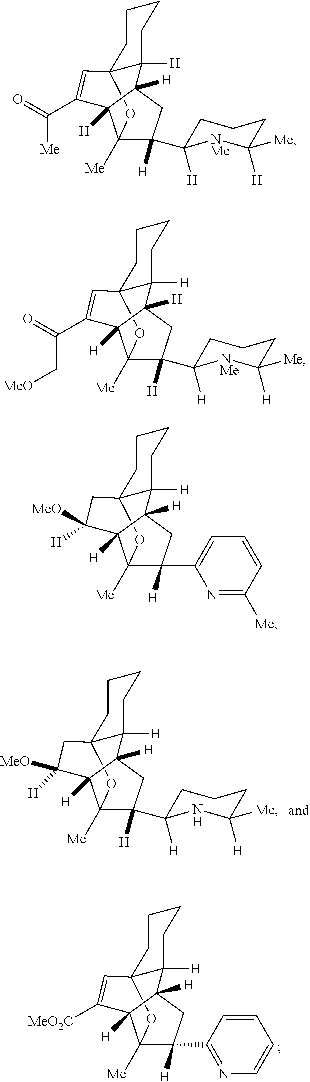

or a pharmaceutically acceptable salt thereof.

### Embodiment 41

A method of agonizing an opioid receptor in a subject in need of such agonization, comprising administering to the subject a therapeutically effective amount of the compound of Formula (II):

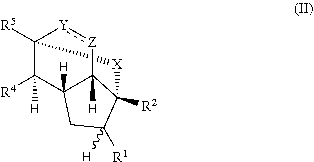

wherein:
[symbol] is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷; Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
   each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇cycloalkyl), —NMsPh, and —C(═O)OH;
R² is H, C₁-C₆ alkyl, (C₁-C₆ alkyl)-C(═O)—, hydroxy, or —O—(C₁-C₆ alkyl);
R⁴ and R⁵ are each independently H or C₁-C₆ alkyl;
   or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
   or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
   or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
   or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
   or R⁶ and R⁷ together form monocyclic or bicyclic C₃-C₁₄ cycloalkyl or C₃-C₁₄ heterocycloalkyl, each individually and optionally substituted with 1-3 R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl);
   or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
   each R^{p} independently is H, C₁-C₆ alkyl, (C₁-C₆ alkyl)-C(═O)—, —Si(C₁-C₆ alkyl)₃, —S(═O)₂—(C₁-C₆ alkyl), or —S(═O)₂—(C₆-C₁₀ aryl);
   R^{q}, R^{s}, and R^{t} are each independently H or C₁-C₆ alkyl;
   wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
   wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
   or a pharmaceutically acceptable salt thereof.

### Embodiment 42

The method according to embodiment 41, wherein, in Formula (II):
[symbol] is a double bond;
X is O;
Y is CR⁶;
Z is CR⁷;
R¹ is C₆-C₁₀ aryl or C₅-C₁₀ heteroaryl, optionally substituted with 1-3 substituents independently selected from the group consisting of C₁-C₆ alkyl, halo, —OH, —O—C₁-C₆ alkyl, —CN, —NO₂, —CF₃, methylene cyclopropyl, and —C(═O)OH;
R² is C₁-C₆ alkyl;
R⁴ and R⁵ together with the carbon atoms to which they are shown attached form a C₃-C₇ cycloalkyl, which is optionally substituted with 1-2 substitutents independently selected from the group consisting of C₁-C₆ alkyl, halo, —OH, —O—C₁-C₆ alkyl, —CN, —NO₂, —CF₃, and —C(═O)OH;
R⁶ is H; and
R⁷ is —C(═O)—(C₁-C₆ alkyl).

### Embodiment 43

The method of according to embodiment 41 or 42, wherein the compound of Formula (II) or a pharmaceutically acceptable salt thereof is the compound of Formula (1):

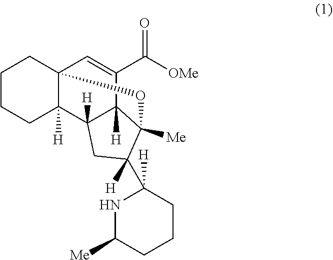 [SMILES: COC(=O)C1=C[C@]23CCCC[C@@H]2[C@H]2C[C@@H]([C@H]4CCC[C@@H](C)N4)[C@@](C)(O3)[C@@H]12]

or a pharmaceutically acceptable salt thereof.

### Embodiment 44

The method according to any one of embodiments 41-43, wherein the opioid receptor is a kappa-opioid receptor (KOR).

### Embodiment 45

A method of treating pain, itching, depression or dissociative hallucination in a subject in need of such treatment comprising administering to the subject a therapeutically effective amount of the compound of Formula (II):

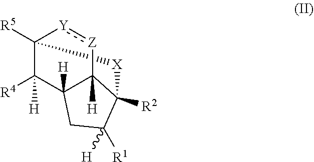

wherein:
[symbol] is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷;
   or R⁶ and R⁷ together form monocyclic or bicyclic C₃-C₁₄ cycloalkyl or C₃-C₁₄ heterocycloalkyl, each individually and optionally substituted with one or more R when [symbol] is a single bond;
Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
   each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇cycloalkyl), —NMsPh, and —C(═O)OH;
R² is H, C₁-C₆ alkyl, (C₁-C₆ alkyl)-C(═O)—, hydroxy, or —O—(C₁-C₆ alkyl);
R⁴ and R⁵ are each independently H or C₁-C₆ alkyl;
   or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
   or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
   or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
   or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
   or R⁶ and R⁷ together form monocyclic or bicyclic C₃-C₁₄ cycloalkyl or C₃-C₁₄ heterocycloalkyl, each individually and optionally substituted with 1-3 R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl); or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
each R^{p} independently is H, C₁-C₆ alkyl, —C(═O)—(C₁-C₆ alkyl), —Si(C₁-C₆ alkyl)₃, —S(═O)₂—(C₁-C₆ alkyl), —S(═O)₂—(C₆-C₁₀ aryl), or —NHTs;
R^{q}, R^{s}, and R^{t} are each independently H or C₁-C₆ alkyl;
   wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
   wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
   or a pharmaceutically acceptable salt thereof.

### Embodiment 46

The method according to embodiment 45, wherein, in Formula (II):
[symbol] is a double bond;
X is O;
Y is CR⁶;
Z is CR⁷;
R¹ is C₆-C₁₀ aryl or C₅-C₁₀ heteroaryl, optionally substituted with 1-3 substituents independently selected from the group consisting of C₁-C₆ alkyl, halo, —OH, —O—C₁-C₆ alkyl, —CN, —NO₂, —CF₃, methylene cyclopropyl, and —C(═O)OH;
R² is C₁-C₆ alkyl;
R⁴ and R⁵ together with the carbon atoms to which they are shown attached form a C₃-C₇ cycloalkyl, which is optionally substituted with 1-2 substitutents independently selected from the group consisting of C₁-C₆ alkyl, halo, —OH, —O—C₁-C₆ alkyl, —CN, —NO₂, —CF₃, and —C(═O)OH;
R⁶ is H; and
R⁷ is —C(═O)—(C₁-C₆ alkyl).

The application further provides the method according to Embodiment 46, wherein the compound of Formula (II) or a pharmaceutically acceptable salt thereof is selected from the group consisting of the enantiomers (non-superimposable mirror image stereoisomers) of the compounds of Embodiment 34, or a pharmaceutically acceptable salt thereof.

### Embodiment 47

The method according to embodiment 45 or 46, wherein the compound of Formula (II) or a pharmaceutically acceptable salt thereof is the compound of Formula (1):

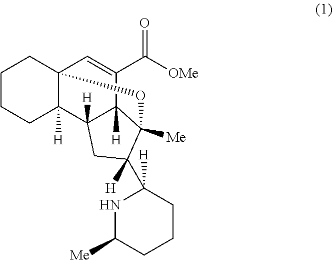 [SMILES: COC(=O)C1=C[C@]23CCCC[C@@H]2[C@H]2C[C@@H]([C@H]4CCC[C@@H](C)N4)[C@@](C)(O3)[C@@H]12]

or a pharmaceutically acceptable salt thereof.

### Embodiment 48

A compound of formula I, having the structure of any one of the group consisting of:

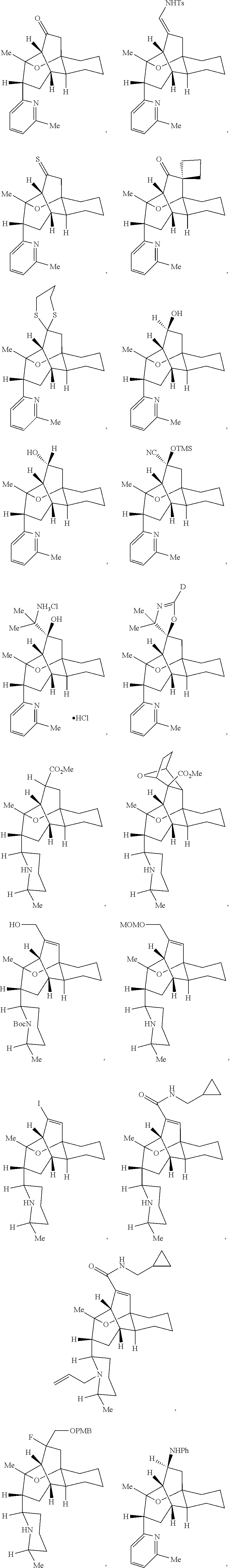

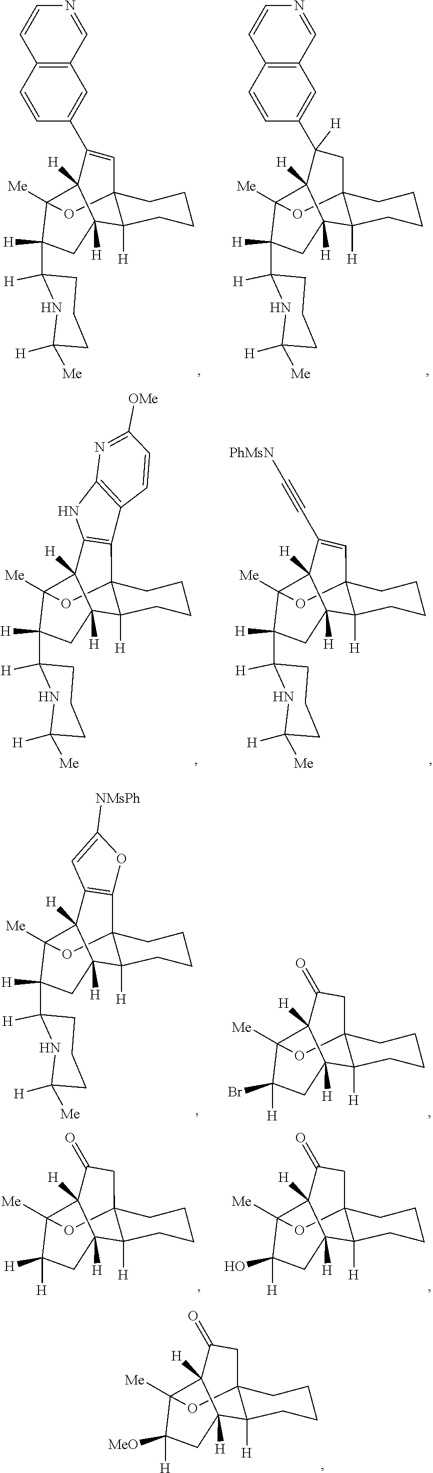

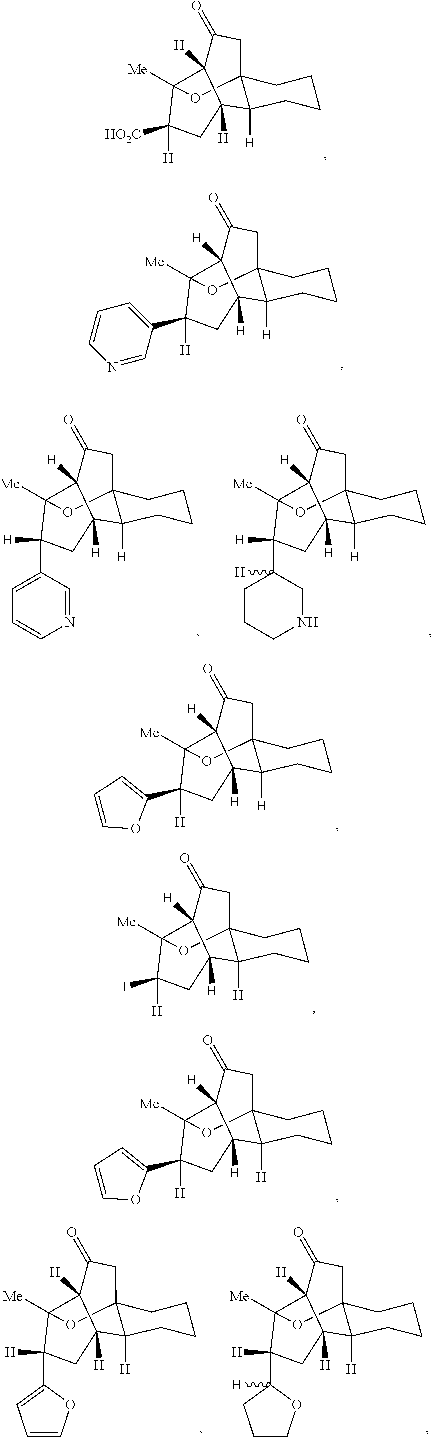

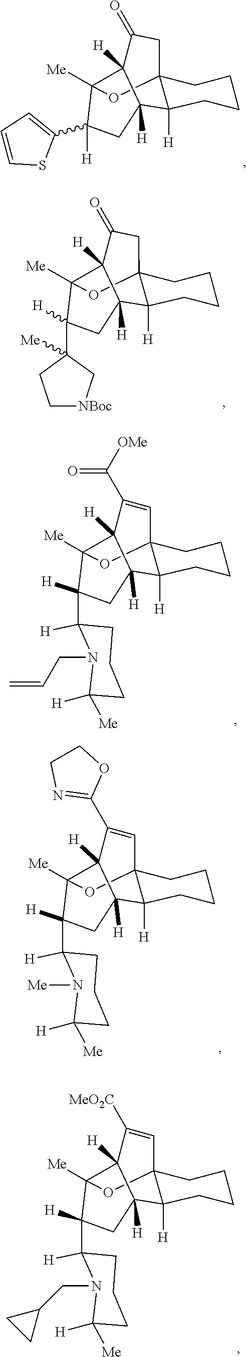

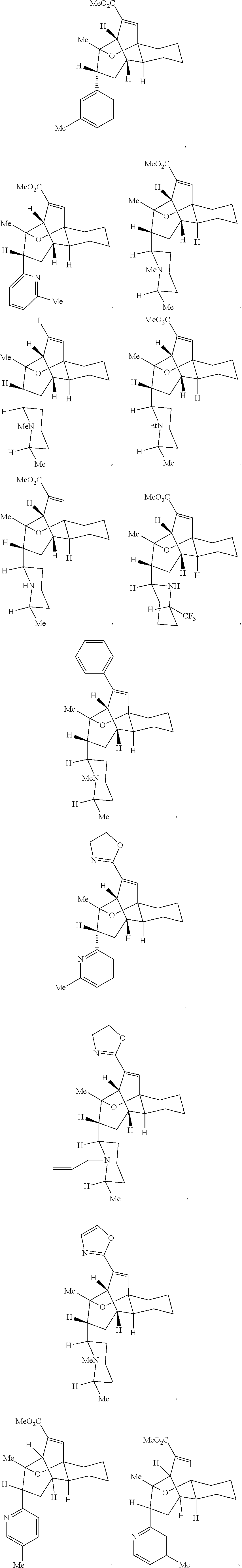

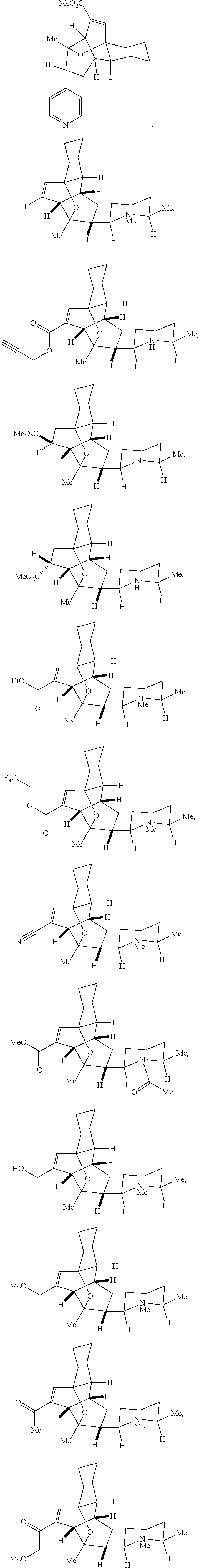

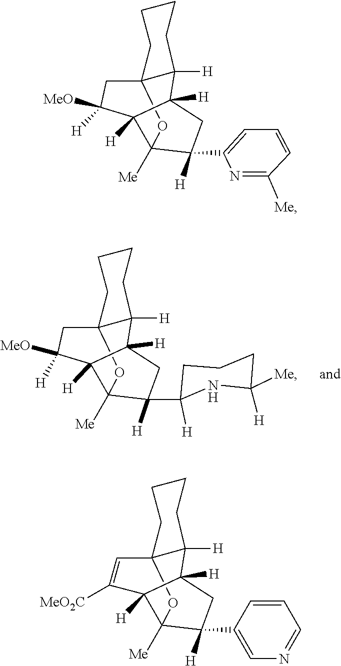

including any enantiomers, scalemic or racemic mixtures, and pharmaceutically acceptable salts thereof.

### Embodiment 49

A method of treating pain, itching, depression or dissociative hallucination in a subject in need of such treatment comprising administering to the subject a therapeutically effective amount of a compound of claim 48, including enantiomers, scalemic and racemic mixtures, and pharmaceutically acceptable salts thereof.

### Embodiment 50

Any process, method or compound as disclosed herein.

### Administration and Pharmaceutical Composition

In general, the compounds described herein will be administered in a therapeutically effective amount by any of the accepted modes of administration for agents that serve similar utilities. Therapeutically effective amounts of a compound described herein may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound, i.e., the active ingredient, will depend upon numerous factors such as the severity of the disease to be treated, the age and relative health of the patient, the potency of the compound being utilized, the route and form of administration, and other factors.

In general, compounds described herein will be administered as pharmaceutical compositions by any one of the following routes: oral, systemic (e.g., transdermal, intranasal or by suppository), parenteral (e.g., intramuscular, intravenous, intrasternal or subcutaneous) topical (e.g., application to skin) administration, or through an implant. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound described herein in combination with at least one pharmaceutically acceptable carrier/excipient. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be chosen from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound described herein in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 20th ed., 2000).

The level of the compound in a formulation can vary within the full range employed by those skilled in the art. Typically, the formulation will contain, on a weight percent (wt %) basis, from about 0.01-99.99 wt % of a compound described based on the total formulation, with the balance being one or more suitable pharmaceutical excipients. Preferably, the compound is present at a level of about 1-80 wt %.

A compound described herein may be used in combination with one or more other drugs in the treatment of diseases or conditions for which a compound described herein or the other drugs may have utility, where the combination of the drugs together are safer or more effective than either drug alone. Such other drug(s) may be administered, by a route and in an amount commonly used therefore, contemporaneously or sequentially with a compound described herein. When a compound described herein is used contemporaneously with one or more other drugs, a pharmaceutical composition in unit dosage form containing such other drugs and a compound described herein is preferred. However, the combination therapy may also include therapies in which a compound described herein and one or more other drugs are administered on different overlapping schedules. It is also contemplated that when used in combination with one or more other active ingredients, a compound described herein and the other active ingredients may be used in lower doses than when each is used singly.

Accordingly, a pharmaceutical composition described herein also can include those that contain one or more other active ingredients, in addition to a compound described herein.

### EXAMPLES

### General Examples for the Processes and Compounds of the Invention

As set forth in the background section, access to Access to GB18 via chemical synthesis poses significant challenge due to its unprecedented polyketide-derived ring structure, strained ether and stereogenic attached-ring motif that positions the piperidine moiety on the hindered, concave face of the oxa-tetracycle. Retrosynthetic cleavage of the attached-ring bridge to a β-haloether/pyridine pair reduces complexity by 28% (448 to 325 mcbits), but control of bridgehead stereochemistry is non-obvious. The attached-pyridine (2) can undergo facile bond rotation to expose either face for hydrogenation. Stereoselective appendage of the pyridine onto the carbocyclic core should overcome the steric repulsion of the endo-face and the tendency of the bridging ether to fragment by ketone E1cB elimination or organometallic E2 elimination. Solutions to both of these problems were unavailable from prior literature and required significant experimentation (vide infra). The potential simplicity offered by this approach of the present invention, however, mitigated its inherent risk. Cross-coupling substrate 3 might be accessed by haloetherification, which keyed the use of a Danheiser annulation transform to the unencumbered convex face of enone 4. The effectiveness of this design is established by execution on large scale to procure large quantities of GB18. Access to nat-GB18 allowed its annotation as a potent antagonist for kappa-(10 nM IC₅₀) and mu-opioid (12 nM) receptors.

Enone 4 was available in 4 steps from 5, the Robinson annulation product of cyclohexanone and methyl vinyl ketone. Conjugate borylation using catalytic Cu(I)-JohnPhos was competent to access cis-decalone stereoselectively in one step, but a two-step protocol of hydrogen peroxide-mediated epoxidation¹⁵ and palladium-catalyzed hydrogenation¹⁶ could be easily scaled to access 29.4 grams (175 mmol) of crystalline and highly pure 5. Use of 1:1 EtOAc:HFIP was important to minimize catalyst loading (1% Pd/C vs. 10% in EtOAc alone), prevent overreduction and ensure reproducible yields. Desaturation was achieved by regioselective (95:5 Δ^{2,3}: Δ^{1,2}) double silylation, followed by catalytic Saegusa oxidation. Although high yields of 4 required 10 mol % Pd(OAc)₂, the oxidation could be run in DMSO at 2 M silyl enol ether 11 (initially biphasic) under a balloon of O₂ and directly chromatographed without workup (13:1 SiO₂:crude 11) to provide a 71 wt % solution of the volatile enone 4 in toluene. Danheiser annulation¹⁷ proceeded with excellent stereoselectivity (concave facial addition product not detected) in the presence of 1.4 equiv. TiCl₄ to provide, initially, an alkenylsilane with the tert-alkyl silyl ether still intact. Double desilylation could be effected in situ by the addition of HFIP at −40° C. to provide a 64% yield of 8 (¹H NMR yield), where acidic desilylation is likely assisted by the residual Ti⁴⁺ Lewis acid. Without chromatography, the product (13.4 g in one run) could be obtained in 98% purity (qNMR) via a single recrystallization of crude material. Desilylation in situ proved significantly more effective than a two-step operation of work-up and subjection to TBAF, K₂CO₃/MeOH or BF₃·Et₂O, which caused varying levels of alkene isomerization or alcohol elimination. This sensitivity also frustrated haloetherification with NBS, TBCHD, Bu₄NBr₃ etc. and led to complex mixtures that were difficult to characterize. Iodoetherification using N-iodosuccinimide (NIS) in HFIP proved unique to yield bridging ether 3 with few byproducts even on large scale (5.66 g, 82%).

Cross-coupling of 3 with a suitable attached-ring partner required extensive experimentation to arrive at a scalable, reproducible and stereoselective procedure. We discovered the photosensitivity of 3 early: attempts to leverage photoredox catalysis led to complex mixtures, as did simple irradiation with visible light LEDs. The material darkened noticeably over a few hours if unprotected from ambient light. Metallated pyridines were unsuccessful for cross-coupling as were Minisci conditions for C-centered radical addition to pyridines; both delivered a complex mixture of materials and no discernable 2. Weix cross-electrophile coupling (Zn⁰, NiCl₂·glyme)¹⁸ with 6-bromo-2-picoline (12), however, provided a mixture of both 2 (ca. 5% yield) and undesired epimer 2b. Unlike prior attempts, byproducts were few and easily identifiable, including 6,6′-dimethyl-2,2′-bipyridine (13), protodehalogenated substrate 14, ether fragmentation product 8 (reversion to the Danheiser annulation product) and ether fragmentation product 15. These latter two byproducts reflect the strain imparted by the oxygen bridge, its instability and tendency to eliminate. Insoluble and soluble reductants alike, however, did not substantively increase yield: Mn⁰ and Zn⁰ favored 8 and TDAE¹⁹⁻²⁰ favored 14. No reducing systems perturbed diastereoselectivity, which remained near unity but did not strongly disfavor 2. We recently reported a suite of alkene hydrofunctionalizations that merged, for the first time, metal-hydride hydrogen atom transfer (MHAT) catalysis with canonical nickel cross-coupling cycles.²¹ Although silanes served as hydride sources to form high energy M−H complexes,²² the MHAT catalyst itself appeared necessary for reduction of Ni²⁺ to Ni^{1+,23} Therefore we explored whether Mn, Fe or Co complexes combined with silane could improve the production of 2. The most promising results were delivered by a Mn(dpm)₃/PhSiH₃ reducing system. An exhaustive screen of solvents found DMA to improve the impurity profile versus other solvents but retard reaction rate, whereas PhMe led to fast reactions but more byproducts; a 1:1 mixture of both proved ideal. A high concentration of I⁻ also decreased reaction times from 12 to 3-6 h and 35° C. as reaction temperature provided the fastest rates with the fewest impurities. The amidine ligands reported by Weix²⁴ did not deliver high yields but perturbed stereoselectivity and pointed us towards the best solution, 1H-pyrazole-1-carboxamidine (16) (Praxidine), a commercially available guanidinylating reagent and anti-inflammatory agent, which favored endo-stereoisomer 2 by over 10:1 versus the exo-product 2b. The stereochemical model for capture of the intermediate, unstabilized carbon-centered radical is unclear but may derive from hydrogen bonds between the pyridine-nickel-ligand complex and the Lewis basic oxygen bridge of 3. The reaction could be scaled up to produce 2.9 grams of 2 in a single pass.

This endo-selective cross-electrophile established one of the bridgehead stereocenters of the attached-ring but the piperidine bridgehead carbon represented a different challenge. The pyridine ring underwent free rotation (established by NOE) to expose both prochiral faces, which suggested hydrogenation might prove non-selective. After extensive strategies to lock the conformation of the ring—quantitative pyridine protonation, poly-hydrogen bond donors, chelating Lewis acids—this expectation was met: an equimolar mixture of stereoisomers was generated in each attempt. We found, however, that N-oxidation effectively differentiated prochiral faces of the pyridine and led to effective diastereoselective hydrogenation (6:1 dr) using Rh/Al₂O₃. This ability to selectively react a single face of this rotatable attached-ring system did not result from hindered rotation according to NOE. We suspect that, instead, the two Lewis basic groups (N-oxide, ether) coordinate the metal surface via two-point binding and allow delivery of hydrogen from only one face. The reaction was chemoselective for reduction of the pyridine ring and the resulting hydroxylamine, and did not effectively reduce the ketone until late stages of the reaction.

Having solved the three main challenges of the synthesis—the unprecedented core, the strained ether, the stereogenic attached-rings—completion of the synthesis became straightforward. The ketone was converted via Barton iodination to its corresponding iodoalkene, which could be converted to the native methylcarboxylate using palladium-catalyzed carbonylation. Each step could be scaled to multigram quantities with few changes to the small-scale procedures. The brevity of the route, its simplicity and practicality allowed reproducible production of GB18 by a single chemist. The two enantiomers could be separated by preparative chiral SFC, crystallized to identify the absolute configuration of each antipode and assayed to determine potential central nervous system targets.

GB18 had been singled out as a potential psychotropic principle of Galbulimima sp. due to its inhibition of preening (5 mg/kg) without effect on the pain threshold. Both nat-GB18 and ent-GB18 were therefore screened by the NIMH Psychoactive Drug Screening Program to identify high affinity receptor targets that might affect mood or behavior. Whereas nat-GB18 showed low or statistically-insignificant (p>0.05) binding at 10 μM to >40 common drug targets, it displaced[³H]U-69593 (87%, p≤0.001) from kappa-opioid receptors (KOR) and [3H]DAMGO (85%, p≤0.001) from mu-opioid receptors (MOR). Follow-up TANGO assays identified nat-GB18 as a potent antagonist at both KOR (IC₅₀=10 nM) and MOR (IC₅₀=12 nM)(delta- and nociceptin opioid receptors were not strongly ligated nor were Mas-related GPCRs). ent-GB18 displaced[³H]U-69593 from KOR with moderate affinity (56% at 10 μM, p≤0.001; Kᵢ=1.1 μM) but functioned as an agonist (EC₅₀=ca. 12.5 M).

The identification of MOR and KOR as high-affinity receptors for GB18 represent the first new target assignment for the GB alkaloids in over 35 years, since the identification of himbacine as a muscarinic receptor antagonist. Overall homology among the GB alkaloids illustrates how the relatively small topology differences between himbacine and GB18, both class 1 alkaloids, impart changes to binding affinity among rhodopsin-like GPCRs (M1-5, subfamily A18 vs. opioid receptors, subfamily A4). A correlation between structure and GPCR selectivity is also demonstrated by himbacine vs. Vorapaxar (enantiomeric series, antagonist of PAR1, subfamily A15), and may serve as an organizing principle to classify GB alkaloids by function. This functional organization as a GPCR ligand set, prompted by the synthesis and target assignment of GB18, complements existing characterization by structure and biosynthesis, and may begin to explain the diverse nervous system effects ascribed to the GB alkaloids over 50 years ago. The robust synthesis platform described here allows the exploration of both enantiomeric series, diverse heterocyclic attached-ring analogs and core functional groups—all of which are expected to affect GPCR affinity and selectivity. GB18 itself is freely available to interested parties.

### REFERENCES

1. Rinner, U.; Lentsch, C.; Aichinger, C. “Synthesis of Galbulimima Alkaloids” Synthesis, 2010, 22, 3763.
2. Bhattacharyya, D. “The Galbulimima alkaloids—a new frontier in alkaloid synthesis” Tetrahedron, 2011, 67, 5535.
3. Rinner, U. Galbulimima alkaloids. Alkaloids. Chem. Biol. 2017, 78, 109-166.
4. Thomas, B, “Galbulimima belgraveana (F. Muell) Sprague, Galbulimima agara.” Eleusis: Journal of Psychoactive Plants and Compounds, 1999, 2, 82-88.
4. Thomas, B. Psychoactive Properties of Galbulimima Bark. J. Psychoactive Drugs 2005, 37, 109.
5. Thomas, B. “Galbulimima bark and ethnomedicine in Papua New Guinea” 2006, 49, 57-59.
6. Thomas, B. “Psychoactive plant use in Papua New Guinea” Science in New Guinea, 2000, 25, 33-59.
7. Gilani, H. & Coblin, L. B. “The cardio-selectivity of himbacine: A muscarine receptor antagonist” Naunyn-Schmiedeberg's Archives of Pharmacology 1986, 332, 16.
8. Neumann, K. 1998. “The Synthesis of the Galbulimima Alkaloids Himgravine and Himbacine: Potential Therapeutic Agents for Alzheimer's Disease” TMR-Grants. FMBICT960878. Category 30 (B30).
9. Chackalamannil, S.; Doller, D.; McQuade, R.; Ruperto, V. “Himbacine analogs as muscarinic receptor antagonists-effects of tether and heterocyclic variations” Bioorg. Med. Chem. Lett. 2004, 14, 3967.
10. Heardown, M. J. Expert Opin. Therap. Patents 2002, 12, 863.
12. Wolde Mussie, E.; Ruiz, G. “Method for reducing intraocular pressure in the mammalian eye by the administration of muscarine antagonists” U.S. Class. 514/219, Patent #5716952 (02 10 98).
13. Ritchie, E.; Taylor, W. C. The Galbulimima Alkaloids. In The Alkaloids; 1967; Vol. IX, pp 529-543.
14. Binns, S.; Dunstan, P. J.; Guise, G. B.; Holder, G. M.; Hollis, A. F.; McCredie, R. S.; Pinhey, J. T.; Prager, R. H.; Rasmussen, M.; Ritchie, E.; Taylor, W. C. “The Chemical Constituents of Galbulimima species” Aust. J. Chem. 1965, 18, 569.
15. Klix, R. C.; Bach, R. D. 1,2-Carbonyl Migrations in Organic Synthesis. An Approach to the Perhydroindanones. J. Org. Chem. 1987, 52, 580-586
16. Torii, S.; Okumoto, H.; Nakayasu, S.; Kotani, T. Hydrogenolysis of α,β-Epoxyketone and Ester to Aldol in Pd(0)/HCOOH/Et3N and H2/Pd/C Reduction Media. Chem. Lett. 1989, 1975-1978.
17. Danheiser, R. L.; Carini, D. J.; Basak, A. (Trimethylsilyl)Cyclopentene Annulation: A Regiocontrolled Approach to the Synthesis of Five-Membered Rings. J. Am. Chem. Soc. 1981, 103, 1604-1606.
18. Hansen, E. C.; Li, C.; Yang, S.; Pedro, D.; Weix, D. J. Coupling of Challenging Heteroaryl Halides with Alkyl Halides via Nickel-Catalyzed Cross-Electrophile Coupling. J. Org. Chem. 2017, 82, 7085-7092.
19. Anka-Lufford, L. L.; Huihui, K. M. M.; Gower, N. J.; Ackerman, L. K. G.; Weix, D. J. Nickel-Catalyzed Cross-Electrophile Coupling with Organic Reductants in Non-Amide Solvents. Chem.—A Eur. J. 2016, 22, 11564-11567.
20. Charboneau, D. J.; Huang, H.; Barth, E. L.; Germe, C. C.; Hazari, N.; Mercado, B. Q.; Uehling, M. R.; Zultanski, S. L. Tunable and Practical Homogeneous Organic Reductants for Cross-Electrophile Coupling. J. Am. Chem. Soc. 2021, doi: 10.1021/jacs.1c10932.
21. S. A. Green, T. R. Huffman, R. O. McCourt, V. van der Puyl and R. A. Shenvi, J. Am. Chem. Soc. 2019, 141, 7709-7714.
22. Shevick, S. L.; Wilson, C. V.; Kotesova, S.; Kim, D.; Holland, P. L.; Shenvi, R. A. “Catalytic hydrogen atom transfer to alkenes: a roadmap for metal hydrides and radicals” Chem. Sci. 2020, 12401-12422.
23. S. L. Shevick, C. Obradors and R. A. Shenvi, J. Am. Chem. Soc. 2018, 140, 12056-12068.
24. Hansen, E. C.; Pedro, D. J.; Wotal, A. C.; Gower, N. J.; Nelson, J. D.; Caron, S.; Weix, D. J. “New ligands for nickel catalysis from diverse pharmaceutical heterocycle libraries” Nature Chem. 2016, 8, 1126.

### Synthetic Examples

### Abbreviations

DMF=N,N-dimethylformamide
DMSO=dimethylsulfoxide
dtbbpy=4,4′-di-tert-butyl-2,2′-dipyridyl
Bipy=2,2′-bipyridine
HFIP=1,1,1,3,3,3-hexafluoro-2-propanol
LED=light-emitting diode
NMR=nuclear magnetic resonance
TFA=2,2,2-trifluoroacetic acid
THF=tetrahydrofuran
TMS=trimethylsilyl
OAc=acetate
Ac=acetyl
OTf=—O—S(═O)₂—CF₃;
Tf=Triflyl
OMs=—O—S(═O)₂—CH₃
Ms=Mesyl
Ts=Tosyl
OTs=—O(S═O)₂-p-toluyl
ONf=—O—S(═O)₂—(CF₂)₃—CF₃
PMB=p-methoxy benzyl
OPMB=—O-(p-methoxy benzyl)
MOMO=methoxymethyloxy=—O—CH₂—O—CH₃
—NMsPh=—N(—S(═O)₂—CH₃)(Ph)

### Materials and Methods

All reactions were carried out under positive pressure of argon unless otherwise noted. Glassware was oven-dried at 120° C. for a minimum of 12 hours, or flame-dried with a propane torch under vacuum (<1 torr). Anhydrous tetrahydrofuran (THF) containing 250 ppm BHT (peroxide inhibitor) was purchased from MilliporeSigma/SigmaAldrich. Anhydrous toluene was obtained by passing the previously degassed solvent through an activated alumina column. Other commercially available solvents or reagents were used without further purification unless otherwise noted. Reactions were monitored by thin layer chromatography (TLC) using precoated silica gel plates from EMD Chemicals (TLC Silica gel 60 F254, 250 μm thickness). Flash column chromatography was performed over Silica gel 60 (particle size 0.04-0.063 mm) from EMD Chemicals and activated basic alumina (Brockmann I, 150 mesh) from Acros. Room temperature or ambient temperature in Beckman Building, Lab 420 is 22° C. Organic solvent from crude reaction mixtures and solutions of pure compounds was evaporated on a Büchi Rotavapor R³.

Hexanes (ACS grade), ethyl acetate (ACS grade), diethyl ether (anhydrous ACS grade), dichloromethane (ACS grade), chloroform (ACS grade), and isopropanol (ACS grade) were purchased from Fisher Chemical and used without further purification. Anhydrous tetrahydrofuran, DMF, and acetonitrile were purchased from Sigma-Aldrich. Anhydrous DMSO was purchased from Acros Organics. Anhydrous ethanol was obtained from Pharmco-Aaper.

Commercially available substrates were used without further purification unless otherwise noted. The reactions were monitored by thin layer chromatography (TLC) using precoated silica gel plates from EMD Chemicals (TLC Silica gel 60 F254) or by LC/MS on an Agilent 6120 Quadrupole system with an ESI probe. Flash column chromatography was performed over Silica gel 60 (particle size 0.04-0.063 mm) from Fischer Scientific or Florsil® from Sigma Aldrich or Acros Organics. ¹H NMR and ¹³C NMR spectra were recorded on Bruker DPX-400, a Bruker DPX-500 or Bruker DPX-600 equipped with cryoprobe, and the residual solvent peaks were used as internal standard (CDCl₃: 7.26 ppm H NMR, 77.16 ppm ¹³C NMR, DMSO-d₆: 2.50 ppm H NMR, 39.52 ppm ¹³C NMR, MeOH-d₄: 3.31 ppm ¹H NMR, 49.00 ppm ¹³C NMR;). NMR data is denoted with apparent multiplicities: s=singlet, d=doublet, t=triplet, q=quartet, and combinations thereof.

Quantitative ¹H NMR (qNMR) yield and purity determinations were performed with ns=1, d1=30 s, integrating versus a known quantity of internal standard (e.g. 1,3,5-trimethoxybenzene). Isolated yields refer to >95% pure material unless otherwise noted.

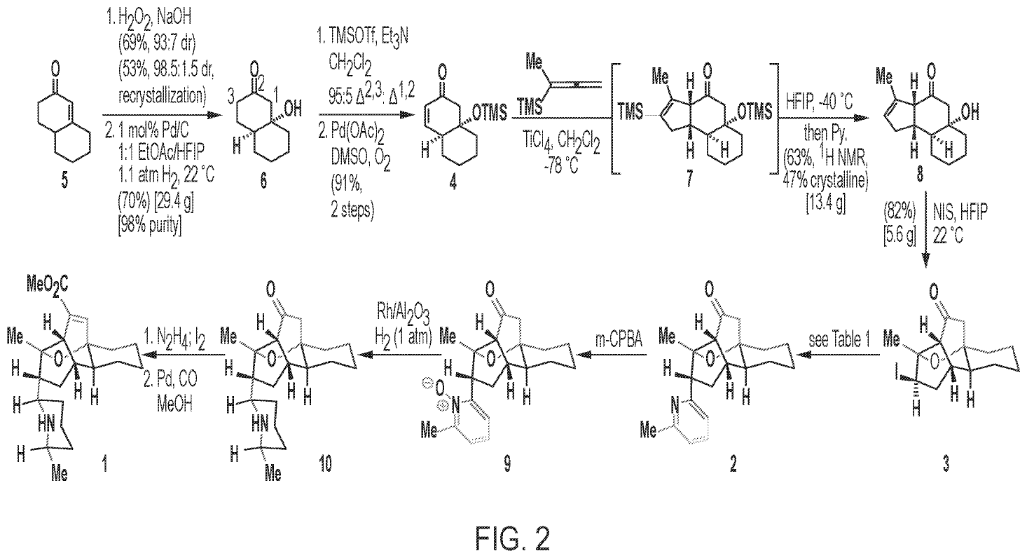

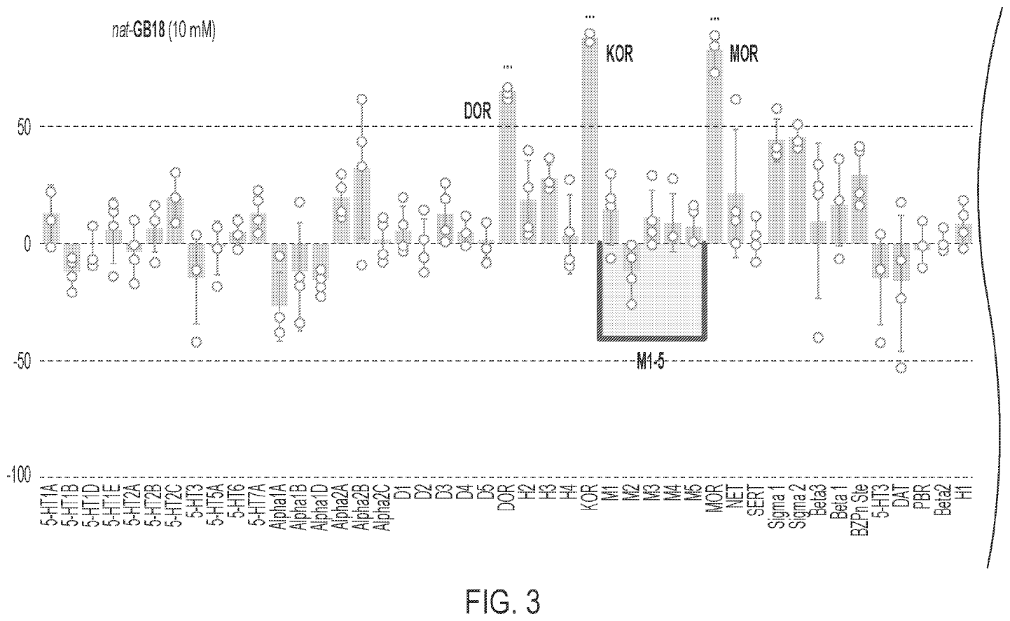

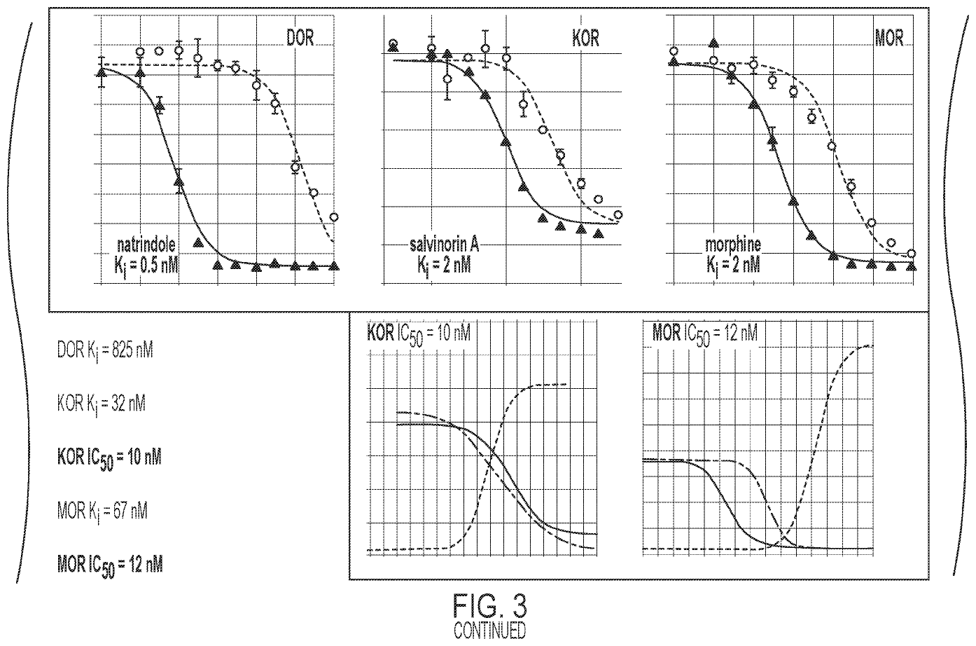

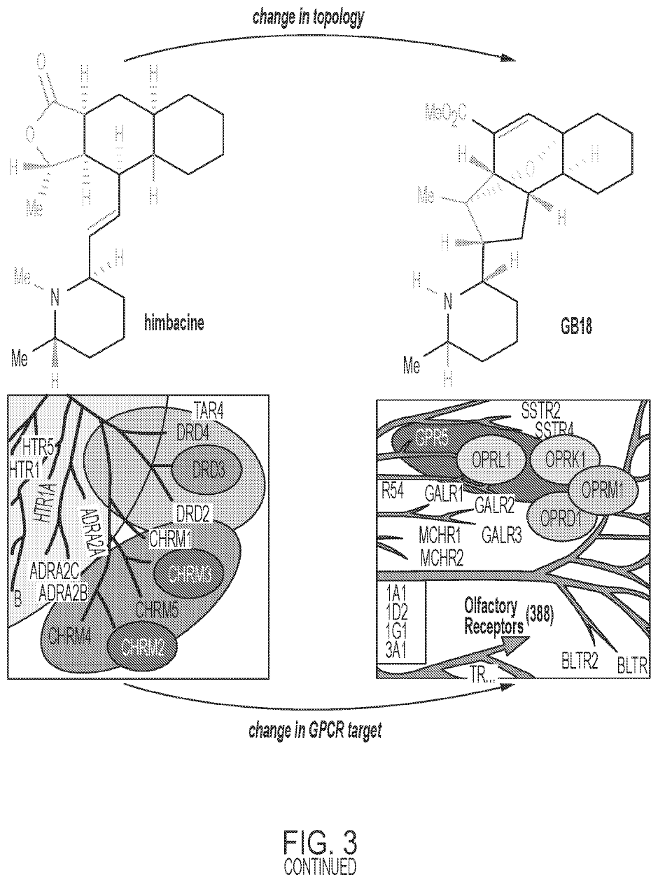

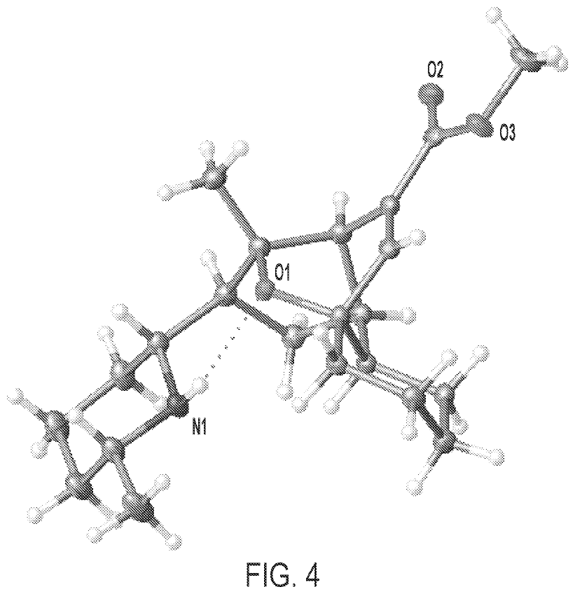

A summary of the synthetic route to GB18 is shown in FIG. 2. The following experimental section should be read in conjunction with FIG. 2.

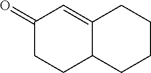 [SMILES: O=C1C=C2CCCCC2CC1]

4,4a,5,6,7,8-hexahydronaphthalen-2 (3H)-one (5)

O═C1CCC(CCCC2)C2=C1

### Robinson Annulation.

To a 2 L round bottom flask equipped with a stir bar was added cyclohexanone (98.2 g, 104 mL, 1.00 mol, 1 equiv.) and toluene (1000 mL) in air. Pyrrolidine (Alfa-Aesar, 131 mL, 1.6 equiv.) was added, giving a turbid mixture and mild exotherm. A Dean-Stark apparatus with condenser was affixed and the mixture was heated at vigorous reflux using an oil bath. The Dean-Stark trap was periodically drained to give, typically, 25-30 mL of a pyrrolidine-containing aqueous phase. When no additional water was condensed (typically 3 h) the Dean-Stark apparatus was replaced with a distillation head and the mixture was concentrated under reflux at atmospheric pressure to remove excess pyrrolidine, giving ca. 800 mL distillate. Additional toluene (200 mL) was added and an additional ca. 200 mL distillate was collected. ¹H NMR analysis of the resulting dark brown solution showed <0.1 equiv. residual pyrrolidine (integration of § 4.32 (bt, J=3.5 Hz, 1H, enamine olefin CH) vs & 2.94 (bs, 4H, pyrrolidine NCH₂)).

The reaction mixture was diluted with additional toluene (900 mL), charged with hydroquinone (550 mg, 5 mmol, 0.005 equiv.), and the hood lights were turned off. Methyl vinyl ketone (Alfa-Aesar, stabilized, freshly distilled from CaCl₂), 81.0 mL, 999 mmol, 0.99 equiv.) was charged to a dropping funnel and added over 30 min without external cooling (final external temperature 43° C.) to give an orange-brown mixture containing a small quantity of fine dark solid. A condenser was affixed to the reaction flask and the mixture was heated to reflux for 16 h. A solution of water (75 mL), AcOH (75 mL), and NaOAc (37.5 g) was added and the reaction was heated to reflux for an additional 4 h. The mixture was allowed to cool to room temperature and the layers were separated. The aqueous layer was extracted with diethyl ether (200 mL), and the combined organic layers were washed with aqueous HCl (2 M, 500+100 mL), saturated aqueous NaHCO₃ (200+100 mL), brine (100 mL), dried over MgSO₄ and concentrated under reduced pressure to give a dark brown oil (133 g). Short path vacuum distillation (pot temperature 105-110° C., head temperature 72-76° C., 1.2-1.3 torr) afforded 111 g (74%) of a clear, pale yellow liquid as a 92.0:7.98 (¹H NMR integration) mixture of conjugated:unconjugated enones (see below). A small portion of this material was subjected to column chromatography (SiO₂, load hexanes, elute 10-15% EtOAc/hexanes) to afford analytical samples of conjugated (5) and unconjugated (SI-1) enones.

¹H NMR (600 MHZ, CDCl₃) δ 5.81 (bs, 1H), 2.47-2.39 (m, 1H), 2.39 (dt, J=16.3, 4.9 Hz, 1H), 2.33-2.25 (m, 1H), 2.29 (ddd, J=16.3, 12.9, 5.1 Hz, 1H), 2.19 (tdt, J=14.3, 13.3, 5.4, 1.5 Hz, 1H), 2.08 (dq, J=13.6, 5.0 Hz, 1H), 1.95 (ddq, J=13.1, 5.5, 2.9 Hz, 1H), 1.90 (ddp, J=13.4, 5.4, 2.7 Hz, 1H), 1.86-1.80 (m, 1H), 1.63 (tdd, J=13.4, 9.2, 4.6 Hz, 1H), 1.48 (qt, J=12.8, 3.3 Hz, 1H), 1.39 (qt, J=13.1, 3.6 Hz, 1H), 1.20 (qd, J=12.9, 3.6 Hz, 1H).

¹³C NMR (151 MHz, CDCl₃) δ 200.3, 167.5, 124.5, 38.1, 36.7, 35.7, 34.6, 29.3, 27.1, 25.7.

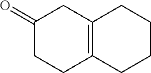 [SMILES: O=C1CCC2=C(CCCC2)C1]

### SI-1

¹H NMR (600 MHZ, CDCl₃) δ 2.73 (bs, 2H), 2.47 (t, J=7.0 Hz, 2H), 2.34-2.28 (m, 2H), 1.96 (bs, 2H), 1.88 (bs, 2H), 1.67-1.58 (m, 4H).

¹³C NMR (151 MHz, CDCl₃) δ 211.5, 128.8, 126.0, 44.6, 39.2, 30.9, 29.9, 29.8, 23.0, 22.6.

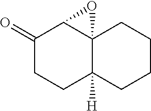 [SMILES: O=C1CC[C@@H]2CCCC[C@@]23O[C@@H]13]

(1aR,4aS,8aR)-hexahydro-3H-naphtho[1,8a-b]oxiren-2(1aH)-one (SI-2)

O═C1CC[C@@](CCCC2)([H])[C@]32[C@H]1O3

### Epoxidation.

To a 2 L three-necked round bottom flask was added a portion of the mixture of enones 5 and SI-1 from the previous step (92.0:8.0 conjugated:unconjugated ratio, 75.1 g, 500 mmol, 1 equiv.), methanol (750 mL) and water (68 mL) under ambient atmosphere. An overhead stirrer and low-temperature thermometer were equipped, and the reaction mixture was cooled to an internal temperature of 0° C. using an ice bath. Hydrogen peroxide solution (50% w/w in H2O, Sigma, stabilized, 85.2 mL, ˜1500 mmol, ˜3 equiv.) was added slowly (slight temperature increase) and stirring on ice was continued, bringing the internal temperature back to 0° C. A 100 mL pressure-equalizing dropping funnel was equipped and charged with aqueous sodium hydroxide (˜6 M, 10.0 g in 42 mL H2O, 250 mmol, 0.5 equiv.). With rapid stirring (max stirrer speed, at least 1500 rpm) and external cooling with an ice bath, approximately 10% of the sodium hydroxide solution was added over 10 min. The internal temperature rose quickly to ˜13° C. at 1-2 min after addition was paused, and gradually fell back to 0° C. over 20 min (see note 1). TLC analysis indicated significant conversion of starting material. The remaining 90% of the sodium hydroxide solution was added over 10 min, with the internal temperature between 3-5° C. At the end of the addition, a ca. 15° C. water bath was applied externally for 1 h, followed by a ca. 20° C. water bath for 2 h. No additional exotherms were observed. The reaction was poured into a mixture of ice (500 mL) and water (500 mL), and extracted with diethyl ether (2×1 L+2× 500 mL). The combined organic layers were washed with saturated aqueous sodium bicarbonate (4×300 mL, see note 2), water (300 mL), brine (200 mL), dried over MgSO₄, and concentrated under reduced pressure to afford a pale-yellow oil (64 g, see note 3). Short path vacuum distillation (pot temperature 92-95° C., head temperature 67-68° C., 0.7-0.75 torr) furnished 57.39 g (69%) of a colourless liquid as a 93:7 cis-:trans-mixture of epoxides (see note 4). The liquid was dissolved in 325 mL of 2% Et₂O/pentane in a 500 mL RBF, seeded with >99:1 dr crystals (50 mg, 0.001 equiv., see note 5), and immediately placed in a −20° C. freezer for 46 h (see note 6). The crystallization flask was placed in an ice-salt bath outside the freezer (−15-−20° C.) and the crystals were broken up quickly with a large spatula. The crystals were collected by vacuum filtration, washed three times with 2% Et₂O/pentane precooled in a −78° C. bath (<−50° C. internal temperature), and dried under vacuum with nitrogen sweep (Org. Synth. 2018, 95, 218-230) to afford 43.93 g (53%) of colourless crystals, ca. 98.5:1.5 dr.

Note 1—Efficient heat dispersion is important during the initial addition of sodium hydroxide solution, else lower yields and dr are obtained.

Note 2—This procedure removes all appreciable quantities of hydrogen peroxide from the organic phase. Washing with sulfur-based reductants gives lower yields upon distillation. The combined aqueous layers contain excess hydrogen peroxide which should be quenched with sodium sulfite or thiosulfate before disposal.

Note 3—Small traces of organic peroxides, which may decompose thermally, are likely present. During distillation, a slow temperature ramp and caution are recommended.

Note 4—Distillation prior to crystallization is required, else high Pd loading is required in the following step.

Note 5—Obtained from a previous run or chromatography (SiO₂, load minimal hexanes, elute 20% Et₂O/hexanes) followed by slow evaporation of a solution of SI-2 in 2% Et₂O/pentane at −20° C. (lightly sealed flask or vial in a −20° C. freezer). Seeding is not strictly necessary but was performed on large scale to avoid reproducibility issues.

Note 6—The product is highly soluble in the crystallization solvent system at room temperature.

¹H NMR (600 MHZ, CDCl₃) δ 3.03 (s, 1H), 2.39-2.30 (m, 1H), 2.25-2.12 (m, 3H), 2.00-1.89 (m, 2H), 1.86-1.73 (m, 2H), 1.47-1.29 (m, 5H)

¹³C NMR (151 MHZ, CDCl₃) δ 206.8, 67.9, 61.7, 36.1, 34.0, 32.3, 30.4, 26.2, 25.7, 21.8.

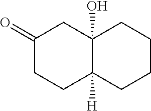 [SMILES: O=C1CC[C@@H]2CCCC[C@]2(O)C1]

(4aS,8aS)-8a-hydroxyoctahydronaphthalen-2(1H)-one (6)

O═C1CC[C@@](CCCC2)([H])[C@@]2(O)C1

### Epoxide Hydrogenation.

A mixture of ethyl acetate (650 mL) and HFIP (650 mL, see note 1) in a 2 L round-bottom flask was sonicated with nitrogen sparging for 30 min. Meanwhile, a 3 L three-necked RBF containing a large football-shaped stir bar was charged with epoxide SI-2 (98.5:1.5 dr, 41.56 g, 250 mmol, 1 equiv.), then evacuated and backfilled with nitrogen (repeated 3×). Pd/C (Sigma-Aldrich, matrix supported, lot #MKCL5758, 5 wt % Pd, 5.32 g, 2.5 mmol, 0.01 equiv.) was added cautiously (see note 2) and the reaction vessel was evacuated and backfilled with nitrogen (repeated 3×). The sparged solvent mixture was cannulated into the reaction vessel under a positive pressure of nitrogen, and the headspace was lightly evacuated (see note 3) and backfilled with nitrogen (repeated 3×). A blast shield was equipped (see note 4) then a H2 balloon with 3-way tap was carefully equipped and the reaction vessel headspace was lightly evacuated (see note 3) and backfilled with H2 (repeated 5×). The depleted balloon was cautiously replaced with a fresh 4-layer balloon of H2, and the reaction was stirred at 1200 rpm for 20 h. ¹H NMR analysis of a reaction aliquot showed ˜1% epoxide remaining. The reaction was purged by flushing the headspace with nitrogen while stirring at 1200 rpm for at least 2 min, then the reaction was filtered over celite with EtOAc (see note 2). The filtrate was concentrated under reduced pressure, azeotroped twice with PhMe, then placed under high vacuum to afford an off-white, wet-looking powder (43.19 g). The product was dissolved in minimal refluxing 60% ethyl acetate/hexanes (250-300 mL required for complete dissolution), seeded with >99:1 dr crystals (20 mg, 0.0005 equiv., see note 5) and allowed to cool over 2-3 h to room temperature, then in an ice bath for 12 h, then at −20° C. for 12 h. The crystals were broken up with a large spatula and filtered. The filter cake was washed with 60% ethyl acetate/hexanes precooled in a −78° C. bath (internal temperature <−50° C.)(washed 3×), then dried at the pump and under high vacuum to afford colourless fine rod-shaped crystals (29.40 g, 98% purity (qNMR), 70% yield).

Note 1—Significant exotherm is observed on mixing. Solvent mixture should be allowed to cool back to room temperature before transfer to reaction vessel.

Note 2—Dry Pd/C sparks readily on exposure to air. Do not allow Pd/C filter cake to dry out during filtration, and dispose of all Pd/C containing residues separately in a water-wet container.

Note 3—Reaction mixture should be exposed to high vacuum until slight foaming is observed then immediately backfilled.

Note 4—Although the flammabilities of the reaction mixture and headspace are diminished by the use of HFIP, exclusion of oxygen and use of a blast shield are highly recommended as a precaution.

Note 5—Seed crystals can be obtained from either a previous batch, or chromatography (SiO₂, load minimal CH₂Cl₂, 33-50% EtOAc/hexanes) followed by slow evaporation from EtOAc.

¹H NMR (600 MHZ, CDCl₃) δ 2.75 (bd, J=14.2 Hz, 1H), 2.39-2.31 (m, 1H), 2.30-2.22 (m, 2H), 2.16 (bd, J=14.2 Hz, 1H), 1.79-1.67 (m, 6H), 1.66-1.55 (m, 2H), 1.52-1.43 (m, 1H), 1.43-1.32 (m, 2H)

¹³C NMR (151 MHz, CDCl₃) δ 211.4, 76.6, 50.3, 41.6, 39.3, 37.7, 28.1, 27.1, 24.5, 23.8.

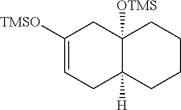

(((4aS,8aS)-4,4a,5,6,7,8-hexahydronaphthalene-2,8a (1H)-diyl)bis(oxy))bis(trimethylsilane)

(SI-3) [TMSO]C1=CC[C@@](CCCC2)([H])[C@@]2(O[Si](C)(C)C)C1

### Silylation.

To a flame-dried 1 L three-necked round bottom flask equipped with a stir bar and low temperature thermometer under argon was added recrystallized alcohol 6 (28.60 g, 170 mmol, 1 equiv.) and dichloromethane (340 mL). The resulting slurry was cooled in an ice bath and triethylamine (118.5 mL, 850 mmol, 5 equiv.) was added. Trimethylsilyl triflate (TMSOTf) (76.96 mL, 425 mmol, 2.5 equiv.) was added over 50 min via syringe, during which time the internal temperature remained between 2-6° C. The ice bath was removed and the clear dark yellow-orange solution was allowed to warm in ambient air. ¹H NMR analysis (C₆D₆, see note 1) of a quenched (sodium bicarbonate, sat. aq.) aliquot taken at 1 h following complete addition of trimethylsilyl triflate indicated complete conversion. At 2 h following complete addition of trimethylsilyl triflate, the reaction was placed in an ice bath and diluted with ice-cold sodium bicarbonate (sat. aq., 400 mL). The aqueous phase was extracted with ether (800 mL, then 2×100 mL), and the combined organic layers were washed with water (200 mL), brine (200 mL), dried over sodium sulfate, and concentrated under reduced pressure to give a light orange-yellow oil containing a small quantity of immiscible dark red droplets. The oil was carefully transferred to a 500 mL round bottom flask, washing with a small amount of pentane and avoiding transfer of the dark red impurities, then concentrated in vacuo (0.3 torr, 23° C.) for <30 min to give a light orange-yellow oil (53.41 g) containing <1 mol % putative Et₃N-derived impurities (determined by total integration of the ¹H NMR peaks δ 0.9-1.1 ppm, t, 9H, assigned to NCH₂CH₃, see note 2). A small portion of material was removed and could be subjected to column chromatography (Florisil®, load hexanes, elute hexanes-5% Et₂O/hexanes) to furnish material for characterization (see note 1). The remaining oil was directly subjected to the subsequent Saegusa reaction.

Note 1: Compound 6 is acid-sensitive; NMR analysis should be conducted in CDCl₃ that was freshly passed through basic alumina or C₆D₆.

Note 2: High levels of Et₃N-derived impurities necessitate higher loadings of Pd(OAc) 2 in the following step.

¹H NMR (600 MHZ, C₆D₆) δ 4.88 (m, 1H), 2.58 (bd, J=15.7 Hz, 1H), 2.45 (ddt, J=17.6, 3.1, 1.6 Hz, 1H), 2.13 (bd, J=17.6 Hz, 1H), 1.75 (dbm, J=16.4 Hz, 1H), 1.67 (dbt, J=12.8, 4.4 Hz, 1H), 1.63-1.40 (m, 5H), 1.31-1.20 (m, 2H), 1.20-1.12 (m, 1H), 0.22 (s, 9H), 0.19 (s, 9H).

¹³C NMR (151 MHZ, C₆D₆) δ 147.1, 100.9, 75.9, 41.4, 39.8, 38.9, 29.3, 27.7, 24.8, 24.3, 2.9, 0.5.

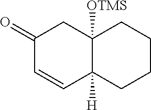

(4aS,8aS)-8a-((trimethylsilyl)oxy)-4a,5,6,7,8,8a-hexahydronaphthalen-2(1H)-one (4)

O═C₁C═C[C@@](CCCC2)([H])[C@@]2(O[Si](C)(C)C)C1

Saegusa oxidation. To a 500 mL round-bottom flask containing silyl enol ether SI-3 as described above (assume 170 mmol, 1 equiv.) was added a large football-shaped stir bar. DMSO (85 mL), and Pd(OAc)₂ (3.817 g, 17 mmol, 0.1 equiv.). The biphasic mixture was sonicated briefly to dissolve Pd(OAc)₂, giving a turbid top layer and a dark red-brown bottom layer. A rubber septum, 18-gauge 3-inch needle fed by a 4-layer balloon filled with O₂ gas, and an 18-gauge needle outlet were equipped, and the reaction was stirred at 1200 rpm for 5 min. The balloon was replaced with a fresh 4-layer O₂ balloon and the vent needle was removed. The reaction was stirred at 1200 rpm for 14 h, after which ¹H NMR analysis of a reaction aliquot showed no discernible starting material remaining. The reaction was loaded onto a dry SiO₂ frit (7 cm height×16 cm diameter) topped with filter paper, transferring with minimal dichloromethane. The frit was eluted with hexanes (500 mL) and the filtrate (containing ˜1.5 g mixed enone and non-polar impurities) was set aside. Further elution with 50% ether in hexanes (3 L), concentration and co-evaporation with PhMe (3×) afforded enone 4 as a pale yellow liquid (51.99 g, 70.94 wt % solution in toluene, 91% yield over two steps from hydroxyketone 6 by ¹H NMR vs 1,3,5-trimethoxybenzene as internal standard). Submitting a small portion of this material to column chromatography (SiO₂, load hexanes, elute 20% Et₂O/hexanes) afforded analytically pure 4.

¹H NMR (600 MHZ, CDCl₃) δ 6.75 (dd, J=10.1, 4.1 Hz, 1H), 5.97 (dd, J=10.1, 1.7 Hz, 1H), 2.70 (d, J=16.0 Hz, 1H), 2.49-2.40 (m, 1H), 2.43 (d, J=16.0 Hz, 1H), 2.02-1.95 (m, 1H), 1.77-1.63 (m, 2H), 1.57-1.43 (m, 2H), 1.43-1.25 (m, 3H), 0.09 (s, 9H).

¹³C NMR (151 MHZ, CDCl₃) δ 199.5, 153.2, 128.4, 76.4, 49.3, 45.8, 37.0, 29.2, 23.8, 23.0, 2.7.

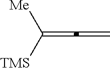

buta-2,3-dien-2-yltrimethylsilane (SI-4)

C═[C]═C(C)[Si](C)(C)C

Allene SI-4 can be synthesized by the procedure of Danheiser and coworkers, but consistently gave yields lower than those previously reported (˜35-53% vs 72-75%) in our hands. Additionally, we have found the intermediate propargyl mesylate is sensitive to long incubation even at low temperature, as well as to CuBr/LiBr purity; these considerations are important to minimize formation of 3-trimethylsilyl-2-propargyl chloride/bromide (up to 10-15 mol % impurity) which was extremely difficult to separate from desired product allene. Use of allene that contained residual 3-trimethylsilyl-2-propargyl chloride/bromide in the subsequent Danheiser annulation does not seem to give appreciably different reaction profiles to runs using propargyl halide-free allene (by TLC) but is deleterious to product stability following workup, particularly on large scale.

Using literature prep (Danheiser and coworkers, Org. Synth. 1988, 66, 1) with following modifications:

CuBr purification. Commercial CuBr (Alfa, dull forest green in appearance or Oakwood, whitish green in appearance; 135 g) was ground into a fine homogenous powder with a pestle (no mortar necessary, can use large weigh-paper, see note 1). The powdered CuBr was added to a large Erlenmeyer flask (min 4 L, ideally larger) and dissolved in concentrated aqueous HBr (48%, typically 600-650 mL, see note 2). The resulting dark purple solution was diluted with water (3.3 L) to give a dull green mixture with some white precipitate. The mixture was split between 2-3 additional large vessels and diluted with copious water (ca. 8-10 L total) to effect complete precipitation, as signalled by a light blue supernatant which does not change in hue on further dilution with water (see note 3). The combined mixtures were allowed to settle and most of the supernatant was decanted. The remaining mixture was vacuum-filtered through a foil-covered fritted glass column (24-30 cm length, 4-5 cm diam) containing 24/40 joints at both ends, then washed continuously with water until the filtrate was colorless. Further washing was conducted with absolute ethanol, then diethyl ether, ensuring that the top of the filter cake was not exposed to air from the start of water washing until the ether wash was completed. The resulting dense white filter cake containing a thin, crumbly top layer of peach-colored solid was dried under vacuum with nitrogen sweep (Org. Synth. 2018, 95, 218-230) and light external heating (heat gun on low setting) applied to the side of the column. When the filter cake was warmed to room temperature, the peach-colored solid was removed and discarded, and the filter cake quickly transferred to a 125 mL round-bottom flask with minimal exposure to air and light (ca. 100 g white solid). The CuBr was dry-stirred under high vacuum (<1 torr) in a 130° C. oil bath for at least 24 h to afford an off-white, faintly yellow solid which was immediately used for the cuprate formation.

Note 1—without crushing, impure CuBr is difficult to completely dissolve in concentrated HBr. Complete dissolution prior to precipitation is required for high purity CuBr.

Note 2—there should be no clumps of solid remaining; if so, add extra concentrated HBr to effect complete dissolution.

Note 3—the precipitated CuBr appears as a white solid which appears stable to ambient laboratory atmosphere and light during the timescale of purification (30 min-1 h) prior to washing with water. If concentrated supernatant is allowed to evaporate, a purple residue can be observed.

LiBr purification. LiBr was dry-stirred under high vacuum (<1 torr) in a 130° C. oil bath for at least 24 h immediately prior to the cuprate formation.

Mesylation. A 2 L three-necked round-bottom flask containing a magnetic stir bar, nitrogen inlet adapter, low-temperature thermometer and a 250 mL pressure-equalizing dropping funnel with rubber septum was heated with a torch under high vacuum and backfilled with dry nitrogen. The flask was charged with 3-trimethylsilyl-2-propyn-1-ol (93.5 mL, 80.9 g, 631 mmol, 1 equiv.) and tetrahydrofuran (640 mL) and then cooled in an ice bath. The dropping funnel was charged with methylmagnesium chloride solution (avg. 3.03 M in THF, 210 mL, 637 mmol, 1.01 equiv.), which was added at such a rate that the internal temperature did not rise above 10° C. (held between 4-8° C.; total addition time 2 h). The near-colorless solution turned pale yellow near the end of addition. Stirring was continued at 0° C. for 80 min during which time the reaction mixture became slightly grey, then the reaction was brought below an internal temperature of −75° C. with a dry ice-acetone bath. Methanesulfonyl chloride (48.76 mL, 630 mmol, 1 equiv.) was added over 10 min via syringe, keeping the internal temperature below −75° C. (mild exotherm). After 2 h the cold bath was depleted of dry ice and the internal temperature rose to between −10-−40° C. over the course of 2.5-3 h.

Cuprate formation. During the maintenance period following methanesulfonyl chloride addition, a 3 L three-necked round-bottom flask was separately equipped with two rubber septa and a 250 mL pressure-equalizing dropping funnel with nitrogen inlet adapter. The flask was quickly charged with anhydrous copper (I) bromide (94.89 g, 630 mmol, 1 equiv.) and anhydrous lithium bromide (57.45 g, 630 mmol, 1 equiv.), and the contents were flamed briefly under vacuum. After 20-30 min the flask was backfilled with nitrogen and an overhead stirrer was equipped. Dry tetrahydrofuran (670 mL) was added, and the resulting green solution containing a small amount of undissolved solid was cooled in an ice bath. Methylmagnesium chloride solution (avg. 3.03 M in THF, 210 mL, 637 mmol, 1.01 equiv.) was added to the reaction mixture over the course of ca. 60 s with vigorous stirring (overhead stirrer maximum rpm). After a further 30 min of stirring at 0° C. a viscous yellow-green suspension was obtained, and the ice bath was replaced by a dry-ice acetone bath.

Allene synthesis. During the cuprate aging period following the second Grignard addition described above, the mesylate solution was equipped with a 20° C. water bath with constant stirring for ca. 15 min, then an ice bath was equipped, such that the reaction spent a total time of 10 minutes between 10-20° C. (internal temperature). The reaction became a clear darkish-yellow/gold in appearance while between 10-20° C. and slight turbidity was observed on subsequent cooling. The mesylate slurry was transferred, via a 16-gauge cannula and with constant stirring, into the well-stirred, cooled cuprate mixture over 45 minutes. The internal temperature of the mesylate mixture was 4° C. during the period of transfer. The green reaction mixture was stirred at −78° C. for 1 h then allowed to warm to room temperature over the course of 11 h. The reaction, now grainy black in appearance, was poured into a 4 L Erlenmeyer flask containing a magnetically stirred mixture of saturated aqueous ammonium chloride (800 mL), water (400 mL), and pentane (800 mL). The well-stirred mixture turned biphasic blue and bronze briefly, then gradually equilibrated to a biphasic turbid organic layer and clear forest green aqueous layer over the course of 1-2 min. The organic layer was washed with ammonium chloride (sat. aq.)(3×400 mL, see note 1), water (12×2 L, see note 2), brine (200 mL), dried over sodium sulfate, then partially concentrated under reduced pressure carefully (200 torr, 22° C.) to give a pale yellow liquid (74.6 g), containing no discernible 3-trimethylsilyl-2-propargyl halide impurities by ¹H NMR. The liquid was distilled through a 12 cm (14/20 connections and diameter) Vigreux column at atmospheric pressure to give 44.27 g (93.7 wt %, 53% yield, see note 3) of allene (SI-4) as a colorless liquid, bp 111-115° C. (see note 4).

Note 1: The organic layer turned pale yellow over the course of washing.

Note 2: This procedure removes tetrahydrofuran from the organic phase.

Note 3: Weight purity was determined by ¹H NMR relative to 1,3,5-trimethoxybenzene as internal standard. The remaining mass balance does not affect the subsequent Danheiser annulation.

Note 4: an additional 3-5% of allene remained in mixed fractions containing a high-boiling impurity, that is deleterious for the subsequent reaction, following vacuum distillation of the pot residue. Careful fractional vacuum distillation can likely recover this material but was not attempted by us.

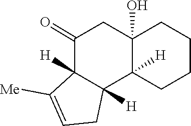

(3aR,5aS,9aS,9bS)-5a-hydroxy-3-methyl-1,3a,5,5a,6,7,8,9,9a,9b-decahydro-4H-cyclopenta[a]naphthalen-4-one (8)

O═C1[C@](C(C)═CC2([H])[C@]2([H])[C@@](CCCC3)([H])[C@@]3(O)C1

### Danheiser Annulation

The procedure has been conducted on scales between 20-130 mmol (61-70% ¹H NMR yield vs. 1,3,5-trimethoxybenzene). A representative procedure follows:

A flame-dried 3 L three-necked round bottom flask equipped with a rubber septum, an overhead stirrer, and a Claisen adapter with rubber septum and nitrogen inlet was charged with enone 4 (43.69 g, 70.94 wt % solution in toluene, 130 mmol, 1 equiv.), dichloromethane (600 mL) and allene SI-4 (20.06 g, 93.72 wt %, 149 mmol, 1.15 equiv.). A low-temperature thermometer was equipped and the reaction was cooled in a dry ice-acetone bath. TiCl₄ (20.0 mL, 182 mmol, 1.4 equiv.) was added to the vigorously stirred mixture via syringe over 10 min, keeping the internal temperature below −70° C. (see note 1). The dark red solution was maintained at −78° C. for 2 h, then a dry ice-acetonitrile bath was equipped and the reaction maintained between −50-−40° C. for 1 h. Subsequently, the rapidly stirred reaction mixture was kept below −40° C. while a solution of HFIP (118 mL, 1118 mmol, 8.6 equiv.) in PhMe (470 mL) was added by cannula, giving a red mixture containing a fine dispersion of white solid, putatively HFIP ice (see note 2). The reaction mixture was stirred at −40° C. for an additional 10 min, and an ice bath was equipped, whereupon the internal temperature slowly rose to 0° C. over the course of ca. 30 min (see note 3). TLC analysis (30% ethyl acetate in hexanes, unquenched aliquot) showed a mixture containing mainly two products: hydroxyvinyl silane (high R_{f}), and desired product (low R_{f})(both non-UV-active, but purple with anisaldehyde stain). Stirring was continued at 0° C. with TLC monitoring every 30 min. After 4 h at 0° C., TLC analysis indicated that the intermediate hydroxyvinyl silane was almost completely converted and similar in intensity to UV-active polar byproduct(s)(one spot immediately below desired product by TLC, UV-active, purple with anisaldehyde stain). An ice-salt bath was equipped and the internal temperature fell to −20° C. Pyridine (101 mL, 1248 mmol, 9.6 equiv.) was added by cannula at a rate that kept the internal temperature below 0° C. The reaction mixture was allowed to cool back to −20° C., then charged quickly with Et₂O (600 mL, precooled in a dry ice-acetone bath), and ice-cold sodium bicarbonate (sat. aq., 900 mL) (caution: bubbling occurs) with rapid stirring (see note 4), giving a yellow mixture containing white precipitate in the aqueous phase. The cold bath was removed, the mixture was filtered through celite and the aqueous layer extracted with ether (100 mL). The organic layer was washed with 10% aqueous CuSO₄ (10×100 mL), disodium EDTA (sat. aq., 5×100 mL), water (100 mL) and brine (100 mL), then dried over sodium sulfate, co-evaporated with toluene (3×) and dried under high vacuum to afford a pale yellow solid (29.59 g, 64% NMR yield integrating the vinyl H peak (δ 5.49 ppm, m, 1H) against 1,3,5-trimethoxybenzene as internal standard, see note 5). The crude product was dissolved in ether (800 mL) and heated to reflux with a heat gun to give a slightly turbid solution, which was allowed to stand at room temperature undisturbed for 10 min, after which a small amount of wispy tan precipitate had settled to the bottom of the flask. The mixture was filtered through a 3 cm plug of celite, washing with ether (50 mL, see note 6), and the clear filtrate was partially concentrated under reduced pressure. The solution was concentrated under reflux to the minimum soluble volume (ca. 300-400 mL), adjusting the volume with additional ether as required, and the solution allowed to cool undisturbed at room temperature for 5 h, then at 0° C. for 2 h, then at −20° C. for 12 h. The mixture was filtered, and the filter cake was washed with precooled Et₂O (−78° C. bath, internal temperature <−50° C.)(3×), allowed to dry at the pump under N₂ flow, then dried under high vacuum to afford fluffy white crystals (13.42 g, 98% purity (qNMR), 47%). If desired, additional material could be recovered by concentration of the filtrate, filtration through a SiO₂ plug (6 cm height×7 cm diameter, elute Et₂O), then column chromatography (SiO₂, load PhMe, elute 20% EtOAc/hexanes) and a further round of recrystallization.

Note 1: The first 1 equiv. of TiCl₄ should be added within 5 min, and the remaining TiCl₄ added at a slightly slower rate. Significant silylether desilylation of starting material 4 occurs if TiCl₄ is added too slowly. Trimethylsilyl ether desilylation is also competitive with allene conjugate addition at temperatures significantly above −78° C. Moderate heat evolution is observed during the addition of the first 1 equiv. of TiCl₄, and the exotherm becomes stronger for the remaining TiCl₄ portion. Yields may be improved if the TiCl₄ is added directly into the well-stirred reaction without touching the sides of the flask, though this effect was not fully investigated; formation of insoluble red-brown solid could be observed when TiCl₄ was added down the flask walls.

Note 2: Without sufficient stirring or without sufficient volumes of PhMe, HFIP freezes into an insoluble block at −40° C.; significant warming (close to 0° C.) is required before the solid HFIP dissolves appreciably, by which point significant decomposition occurs due to unquenched TiCl₄.

Note 3: The white dispersion completely dissolved during the period of warming to 0° C.

Note 4: More product decomposition is observed if complete mixing of sodium bicarbonate solution with the reaction mixture is slow.

Note 5: The crude product was subjected to high vacuum (<0.5 torr, 23° C.) for at least 24 h prior to trituration and recrystallization to minimize residual pyridine and HFIP, which likely interfere with trituration and recrystallization steps, though this effect was not fully investigated.

Note 6: Small amounts of product partially crystallize on the filtration apparatus during filtration due to evaporative cooling, and can be redissolved with additional ether. A small layer of brown sludge is retained at the top of the celite plug and can be eluted with dichloromethane, but contains negligible product.

¹H NMR (600 MHZ, CDCl₃) δ 5.49 (bs, 1H), 3.13 (bd, J=7.3 Hz, 1H), 2.61 (qd, J=7.8, 3.1 Hz, 1H), 2.53-2.44 (m, 2H), 2.34 (d, J=13.6 Hz, 1H), 2.27 (bd, J=15.7 Hz, 1H), 1.94-1.85 (m, 1H), 1.76-1.60 (m, 3H), 1.68 (s, 3H), 1.53-1.40 (m, 5H), 1.40-1.31 (m, 1H).

¹³C NMR (151 MHz, CDCl₃) δ 211.6, 138.2, 127.0, 74.7, 60.7, 53.0, 44.1, 42.4, 38.9, 35.5, 26.1, 21.9, 21.2, 15.5.

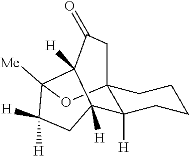

(2R,3aR,5aS,9aS,9bS)-2-iodo-3-methyldecahydro-3,5a-epoxycyclopenta[a]naphthalen-4 (5H)-one (3)

[H][C@@]1(I)C[C@@]2([H])[C@@]3([H])[C@]4(CCCC3)CC([C@@]2([H])C(C)1O4)═O

### Iodoetherification.

To a 500 mL round-bottom flask containing a stirred, faintly yellow solution of hydroxyolefin 8 (4.406 g, 20 mmol, 1 equiv.) in HFIP (100 mL) was added N-iodosuccinimide (5.850 g, 26 mmol, 1.3 equiv.) in one portion. The mixture darkened rapidly to cherry-red as the N-iodosuccinimide dissolved, and iodine appeared to precipitate as fine dark purple crystals over the course of 30 s−1 min. The mixture was stirred for 1 h and an ice bath was equipped. A mixture of Na₂SO₃ (sat. aq., 100 mL) and NaHCO₃ (sat. aq. 40 mL) was added with rapid stirring. The reaction mixture was stirred for an additional 5 minutes and poured into a separatory funnel containing Et₂O (150 mL) and brine (150 mL)(mild exotherm). The layers were separated and the aqueous layer was extracted with additional Et₂O (2×100 mL). The combined organic layers were dried over Na₂SO₄, concentrated under reduced pressure and coevaporated with toluene to give a white-pale yellow residue (11.4 g). The crude product was loaded onto a pre-equilibrated (Et₂O) SiO₂ frit (5 cm height×7 cm diameter) with minimal CH₂Cl₂, eluted with Et₂O (600 mL), and the filtrate was concentrated under reduced pressure to give a pale yellow crystalline solid (7.7 g). Column chromatography (SiO₂, 16 cm height×5.3 cm diameter, wet load minimal PhMe, elute hexanes to 8% EtOAc/hexanes) furnished a white to pale yellow solid (5.77 g). The product was recrystallized by dissolution in refluxing Et₂O then slow concentration (0.5-1 drops per second) on a rotary evaporator (30° C. water bath) set to a slow-moderate rotation speed setting until the crystals which formed were barely covered by solvent. The yellow supernatant was removed by pipette, and the crystals were washed with minimal Et₂O several times (4×1 mL) until all traces of colour were removed. Drying under high vacuum afforded white crystals (5.309 g). The combined washings were concentrated and recrystallized following the procedure above to afford a second crop of crystals (358 mg). Combined isolated yield of 3 was 5.667 g, 82%, with small amounts of additional material in the filtrate (see note 1).

Note 1: Iodoether 3 is slightly unstable under ambient conditions, particularly with exposure to light (irregular formation of brown spots, homogeneous development of a purple tint, and yellowing, variously, between different once-pure solid samples kept at room temperature for several hours or taken in and out of a −20° C. freezer repeatedly). Recrystallization increases longevity of 3 and reproducible reaction outcomes for the subsequent cross-electrophile coupling. We recommend storing recrystallized 3 under argon in a −20° C. freezer in the dark, under which conditions no decomposition is observed for at least several weeks.

¹H NMR (600 MHZ, CDCl₃) δ 4.42 (dd, J=7.7, 7.1 Hz, 1H), 2.60 (d, J=19.4 Hz, 1H), 2.60 (dd, J=13.7, 7.7 Hz, 1H), 2.48 (d, J=4.0 Hz, 1H), 2.48 (ddd, J=13.7, 7.1, 5.5 Hz, 1H), 2.00 (dd, J=19.3, 1.6 Hz, 1H), 1.98-1.95 (m, 1H), 1.75-1.68 (m, 3H), 1.68-1.62 (m, 1H), 1.57 (s, 3H), 1.47-1.36 (m, 2H), 1.36-1.18 (m, 2H), 1.10 (qd, J=13.1, 3.5 Hz, 1H).

¹³C NMR (151 MHz, CDCl₃) δ 211.4, 84.9, 75.3, 56.2, 50.0, 49.7, 43.3, 42.1, 35.7, 33.4, 30.4, 29.0, 25.7, 22.9.

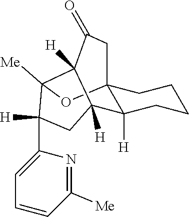 [SMILES: Cc1cccc([C@H]2C[C@H]3C4CCCCC45CC(=O)[C@H]3C2(C)O5)n1]

(2R,3aR,5aS,9aS,9bS)-3-methyl-2-(6-methylpyridin-2-yl) decahydro-3,5a-epoxycyclopenta[a]naphthalen-4 (5H)-one (2)

O═C1[C@]2([H])[C@](C3)([H])[C@@]4([H])[C@@](CCCC4)(OC2([C@@]3([H])C5═CC═CC(C)═N5)C)C1

### Cross-Electrophile Coupling.

NaI (Oakwood Chemical) was dried in a 110° C. oven for at least 12 h before use. Li₂CO₃ (Sigma-Aldrich) was dried in a 110° C. oven for at least 12 h before use. NiBr₂ diglyme (Sigma-Aldrich) was used as received, and stored under argon atmosphere. Mn(dpm) 3 was synthesised and purified according to J. Am. Chem. Soc. 2019, 141, 7709. Ligand SI-5, 1H-pyrazole-1-carboxamidine, HCl salt (Combi-Blocks), was used as received. N,N-dimethylacetamide (DMA)(anhydrous, 99.9%, Sigma-Aldrich) was degassed by sparging with argon in an ultrasonication bath for at least 40 min prior to use for cross-electrophile coupling. 2-iodo-6-methylpyridine SI-6 was synthesised according to Eur. J. Org. Chem. 2002, 4181. Phenylsilane (Oakwood Chemical) was used as received.

### Procedure:

A flame-dried 1 L two-necked round bottom flask equipped with two rubber septa and egg-shaped stir bar was placed under argon, and charged sequentially with NaI (12.72 g, 84.88 mmol, 6 equiv.), Li₂CO₃ (1.045 g, 14.15 mmol, 1 equiv.), 1H-pyrazole-1-carboxamidine hydrochloride (SI-5)(4.147 g, 28.29 mmol, 2 equiv.), NiBr₂ diglyme (10.98 g, 31.12 mmol, 2.2 equiv.), Mn(dpm) 3 (11.76 g, 19.45 mmol, 1.375 equiv.), and recrystallized iodoether 3 (4.897 g, 14.15 mmol, 1 equiv.) prepared according to the procedure described above. The flask was evacuated and backfilled with argon three times, and charged with dry, degassed DMA (430 mL) via cannulation. On scales above 3.3 mmol, during which transfer of DMA took more than a few minutes, small amounts of bubbling were observed as DMA was added. 2-iodo-6-methylpyridine SI-6 (5.11 mL, 42.4 mmol, 3 equiv.) was injected into the stirred mixture. One septum was pierced with a long 18-gauge needle fed by a 4-layer balloon filled with argon, and an 18-gauge needle outlet was equipped. The mixture was stirred at high speed (at least 1200 rpm) for at least 60 min with continual argon sparging (the balloon was replaced with a fresh argon-filled balloon when nearly depleted; typically 2-3 replacements were needed, see note 1). A colloidal, yellow-brown mixture was obtained. PhSiH₃ (6.98 mL, 84.9 mmol, 4 equiv.) was injected with stirring, maintaining positive pressure with an argon balloon (see note 2), and the flask was immediately placed in a pre-heated 35° C. water bath. On this scale the water bath temperature dropped to 30° C. initially and rose back to 35° C. over 10-15 min. This bath temperature was maintained for the remainder of the reaction. The reaction underwent a series of changes in appearance from turbid yellow-brown to turbid yellow-olive to turbid pastel green/light jade to turbid dark jade over 30 min-1 h. Reaction appearance depends on scale and can also be correlated with reaction profile of TLC/LCMS aliquots (see note 3). On scales below 3.3 mmol, a translucent dark yellow-brown/olive mixture free from visible particulate is formed, which persists for 2-8 h, during which apparent iodoether consumption occurs at a roughly constant rate (TLC analysis). Formation of a clear dark forest-green solution is correlated with full consumption of both iodoether (TLC) and iodopyridine (LCMS, see note 4 below), following which the reaction (under these conditions) can usually be left, as is convenient, for at least several hours prior to workup without appreciable product degradation.

On larger scales (>3.3 mmol) slow conversion over 2-8 h followed by stalling (low apparent iodoether 3 consumption (TLC), see note 5) is sometimes observed, correlated with a dark olive reaction mixture which contains visible undissolved particulate. In these cases, it is necessary to add an additional portion of PhSiH₃ (349 μL, 0.2 equiv.) and continuously monitor the reaction (see note 6) until no further conversion occurs. This process is repeated until the uncalibrated LCMS ion ratio of desired product and isomers to unconverted iodopyridine (intensity (m/z=+312)/intensity (m/z=+220)) is greater than 100:1 (typically 2-3 cycles of extra PhSiH₃ addition and 6-8 h later). A further portion of PhSiH₃ (87 μL, 0.05 equiv.) and iodopyridine 10 (85 μL, 0.05 equiv.) are injected simultaneously and reaction monitoring is continued until apparent stalling is observed (see note 7). This process is repeated twice with additional PhSiH₃ (34 μL, 0.02 equiv.) and iodopyridine SI-6 (35 μL, 0.02 equiv.), following which the apparent iodoether 3 spot is faint and similar in color intensity (with anisaldehyde stain) to faster-moving non-polar byproducts.

The reaction is poured into a separatory funnel containing EtOAc (400 mL, 1 volume) and crushed ice (800 mL, 2 volumes), giving an amber yellow organic layer and forest green aqueous layer (aqueous wash 1). The aqueous layer was extracted with EtOAc (6×100 mL, 1.5 volumes total), following which its pH was measured to be ca. 6-7. Full product recovery was confirmed by TLC and aqueous wash 1 was discarded. The combined organic layers, labelled as organic extract 1, were washed with half-saturated brine (2×400 mL, 2 volumes total) then brine (100 mL, 0.25 volumes). These combined aqueous washings (aqueous wash 2) were back-extracted with EtOAc (2×100 mL, 0.5 volumes total). The resulting back-extracts were combined, labelled as organic extract 2, and washed with half-saturated brine (2×100 mL, 0.5 volumes total). Organic extracts 1 and 2 were combined and dried over Na₂SO₄, concentrated under reduced pressure, then under a stream of nitrogen for several hours, to afford a yellow oil, 19.2 g. Two rounds of column chromatography (first round: SiO₂, 16 cm height×8 cm diameter, load PhMe, elute 8-10-20-35-40% EtOAc/hexanes; second round: SiO₂, elute 10% acetone/hexanes, see note 8) afforded desired pyridine SI-6 (2.925 g, 66%) as a white powder (see note 9).

Note 1: Reproducible reaction performance depends on the purity of Mn(dpm)₃, iodopyridine SI-6 and iodoether 3. I₂-contaminated iodopyridine SI-6 can be purified by washing a solution of SI-6 in ether with a mixture of equal volumes of saturated aqueous Na₂SO₃/saturated aqueous NaHCO₃, drying over Na₂SO₄, concentration under reduced pressure and vacuum distillation (64° C., 7 torr) to afford SI-6 as a light orange oil pure by NMR (¹H/¹³C).

Note 2: The reaction can be run without degassing, but this can lead to poor conversion and large variations in reaction outcome between runs, even on small-medium scale. Running the reaction under either air or oxygen atmosphere led to poor conversion and substantial observed hydration product. Attempted spiking with substoichiometric oxygen gas was either detrimental or non-beneficial in our hands.

Note 3: Hydrogen evolution occurs to some extent under these conditions, and may influence reaction kinetics related to Mn/Ni species as noted previously (see SI of J. Am. Chem. Soc. 2019, 141, 7709). We did not observe any benefit running the reaction under hydrogen atmosphere (1 atm or 400 psi (˜27 atm)). Small scale reactions (<0.2 mmol) could be run in sealed tubes. Reactions on larger scale were run under a positive pressure of argon maintained by a 3-/4-layer balloon that was not replaced over the duration of reaction. Septa were immediately resealed by electrical tape if pierced for aliquot removal or reagent addition.

Note 4: Some iodopyridine SI-6 (M+H, m/z=+220) is consumed quickly under these conditions. The iodopyridine homodimerization byproduct 6,6′-dimethyl-2,2′-bipyridine SI-7 (M+H, m/z=+185) is observed in various proportions (uncalibrated LCMS ion ratios) relative to both iodopyridine SI-6 and desired product+isomers (M+H, m/z=+312). Because it is an unproductive (as ascertained by control experiments) and competitive ligand for Ni, its formation served as a parameter to optimize against during the course of reaction development. Undesired formation of SI-7 occurs concomitantly with desired product formation if more silane, and particularly iodopyridine SI-6, are added to revive stalled reactions, necessitating careful control of silane and iodopyridine stoichiometry, both for initial and additional portions.

We did not monitor the formation of iodopyridine protodehalogenation byproduct (M=+93), except very crudely by TLC where it appears as a moderately UV-active spot with slightly lower R_{f} than desired product (30% EtOAc/hexanes).

Note 5: This is usually correlated with a high m/z=+220/+312 ratio (LCMS).

Note 6: The reaction is normally run overnight, and stalling is ascertained the following day. If the reaction has proceeded to completion then workup can commence. Otherwise, further silane/iodopyridine addition is carried out.

Note 7: Some proportion of non-specific decomposition of iodoether to uncharacterized, non-polar byproducts (faster-moving than or copolar to iodoether by TLC) is usually observed when most of the iodoether has been apparently consumed (TLC). Iodoether starting material could not be detected on attempted reisolation of the apparent iodoether spot by column chromatography (SiO₂) following workup. We have never isolated the hypothetical iodoether homodimerization byproduct during the course of reaction development.

Note 8: Sufficient passage through silica is required to remove polymeric silane species (broad ¹H NMR signals at δ ca. 8 and ca. 5 ppm, broad ¹³C NMR signals at δ ca. 140-120 ppm). Alternatively, these and other non-pyridine containing byproducts can be largely removed via an alternative workup: the reaction was diluted with ether (1 volume) and extracted with cold 2 M HCl (3×20 equiv.)(exotherm observed), adding the acidic extract to a slurry of solid Na₂CO₃ (75 equiv) in CH₂Cl₂ (0.2 volumes). The ether layer turned colorless over the course of washing. The basified CH₂Cl₂ mixture was extracted with CH₂Cl₂ several times until no product was detected, dried over Na₂SO₄, concentrated under reduced pressure to remove CH₂Cl₂, blown down with nitrogen to dryness over several hours, and chromatographed directly; or on larger scale was partitioned between EtOAc/water and worked up as per the main workup procedure described above, to remove DMA. Control experiments showed that the desired product is slightly unstable to aqueous HCl (over half conversion standing in 2 M HCl/THF (1:1) over 12 h at room temperature) and we preferred a non-acidic workup procedure on scale for this reason, despite the more cumbersome purification required. If minimization of chromatographic purification is a priority, subjecting the crude product following aqueous workup to the first round of column chromatography described above, slow evaporation of pooled product-containing fractions from EtOAc and washing with ether affords pure 2 as colorless crystals of X-ray quality.

Note 9: Diastereoselectivity, for the specific combination of iodoether 3 and iodopyridine SI-6, was strongly dependent on the use of ligand SI-5 and did not vary significantly with respect to other parameters (including temperature, solvent, base, and additives which were not strong metal-binding ligands).

¹H NMR (600 MHZ, CDCl₃) δ 7.49 (t, J=7.7 Hz, 1H), 7.10 (d, J=7.7 Hz, 1H), 6.99 (d, J=7.7 Hz, 1H), 3.30 (dd, J=11.5, 4.5 Hz, 1H), 2.67 (d, J=19.2 Hz, 1H), 2.54 (s, 3H), 2.44-2.34 (m, 3H), 2.30 (dd, J=12.9 Hz, 4.5 Hz, 1H), 2.16-2.10 (m, 1H), 1.95 (dd, J=19.2, 1.6 Hz, 1H), 1.79-1.68 (m, 3H) 1.65-1.58 (m, 1H), 1.40-1.26 (m, 3H), 1.29 (s, 3H), 1.20-1.09 (m, 1H).

¹³C NMR (151 MHz, CDCl₃) δ 213.5, 158.5, 157.3, 135.9, 120.9, 120.8, 85.8, 74.6, 61.5, 56.1, 48.8, 43.3, 43.2, 39.4, 35.4, 30.5, 26.0, 25.2, 24.8, 23.1.

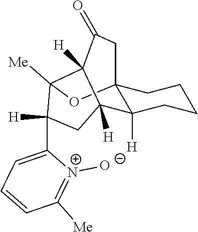 [SMILES: Cc1cccc([C@H]2C[C@H]3C4CCCCC45CC(=O)[C@H]3C2(C)O5)[n+]1[O-]]

2-methyl-6-((2R,3aR,5aS,9aS,9bS)-3-methyl-4-oxododecahydro-3,5a-epoxycyclopenta[a]naphthalen-2-yl)-1λ4-pyridin-1-olate (9)

O═C1[C@]2([H])[C@](C3)([H])[C@@]4([H])[C@@](CCCC4)(OC2([C@@]3([H])C5=CC=CC(C)═[N]5[O—])C)C1

### Pyridine N-Oxidation.

To a stirred solution of pyridine 2 (311.4 mg, 1 mmol, 1 equiv.) in CH₂Cl₂ (wash bottle, 5 mL) in an ice bath was added m-CPBA (ca. 70%, 493.1 mg, ca. 2 mmol, ca. 2 equiv.) in one portion. The ice bath was removed after 10 min and the reaction was allowed to warm unassisted to room temperature. After 2.5 h an additional portion of m-CPBA (ca. 70%, 246.7 mg, ca. 1 equiv.) was added and the reaction stirred for an additional 14 h (see note 1). The reaction was diluted with CH₂Cl₂ (10 mL) and quenched with a mixture of Na₂SO₃ (sat. aq., 5 mL) and NaHCO₃ (sat. aq., 5 mL). The organic layer was washed with NaHCO₃ (sat. aq., 5×3 mL) and brine, dried over sodium sulfate, concentrated under reduced pressure and subjected to high vacuum to afford N-oxide 9 as a crushable, slightly hygroscopic white foam (278 mg, 85%). Higher yields of product contaminated with small amounts (typically 2-3%) of m-CPBA-derived byproducts were obtained using fewer aqueous washes; such material is competent in the subsequent hydrogenation but we recommend using pure material for reproducibility reasons.

Note 1—Apparent stalling at very high conversion (small amounts of starting material by TLC and LCMS (m/z=+312)) is observed after reaction at room temperature for less than an hour.

¹H NMR (600 MHz, CDCl₃) δ 7.81 (dd, J=7.3, 2.9 Hz, 1H), 7.15-7.09 (m, 2H), 4.25 (dd, J=12.1, 5.7 Hz, 1H), 2.84 (ddd, J=13.6, 12.1, 6.2 Hz, 1H), 2.69 (d, J=19.2 Hz, 1H), 2.50-2.48 (m, 1H), 2.49 (s, 3H), 2.16 (bddd, J=6.0, 4.3, 1.8 Hz, 1H), 2.03 (dd, J=19.3, 1.6 Hz, 1H), 1.79-1.69 (m, 3H), 1.69-1.63 (m, 1H), 1.62-1.56 (m, 2H), 1.51-1.42 (m, 1H), 1.42-1.19 (m, 2H), 1.31 (s, 3H), 1.12 (qd, J=13.0, 3.5 Hz, 1H).

¹³C NMR (151 MHZ, CDCl₃) δ 212.1, 152.1, 149.0, 124.3, 124.2, 124.1, 85.8, 74.8, 60.8, 52.6, 46.0, 42.9, 42.7, 41.9, 35.7, 30.2, 25.7, 25.5, 23.0, 18.8.

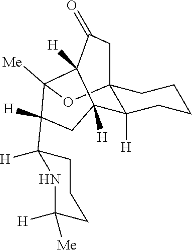 [SMILES: CC1CCCC([C@H]2C[C@H]3C4CCCCC45CC(=O)[C@H]3C2(C)O5)N1]

(2R,3aR,5aS,9aS,9bS)-3-methyl-2-((2R,6R)-6-methylpiperidin-2-yl) decahydro-3,5a-epoxycyclopenta[a]naphthalen-4 (5H)-one (10)

### O═C1[C@]2([H])[C@]3([H])[C@@]4([H])[C@@](CCCC4)(OC2([C@@]([H])([C@@]5([H])CCC[C@@]([H])(C)N5[H])C3)C)C1

### Heteroarene Hydrogenation.

Under ambient atmosphere, N-oxide 9 (64 mg, 0.196 mmol, 1 equiv.), Rh/Al₂O₃ (5 wt % Rh, Oakwood, 126 mg, 0.3 equiv.) and CH₂Cl₂ (10 mL) were charged to a 3 cm diameter test tube and placed in a reactor slot of a Freeslate Junior OSR (Unchained Labs) apparatus. The inlet lines are purged with nitrogen gas and the overhead stirring block, with overhead stirring paddle attachment attached, is placed above the reaction and tightened to seal the apparatus. The stirring is set to 600 rpm and the reaction is purged with H2 gas at 50 psi (3×) followed by pressurization to 400 psi. Stirring at 25° C. was maintained with a H2 pressure range of 395-405 psi, where the pressure dipped to 395 psi several times over the course of the reaction (and was immediately restored to ca. 405 psi when this happened). After 15 h, the reaction was purged with N₂ gas at 100 psi (3×) with stirring, the pressure was released, and the overhead stirring block removed. The reaction was filtered through celite. The filtrate was concentrated and the residue was immediately subjected to the next reaction (see note 1), or for characterisation purposes, could be purified by column chromatography (basic alumina, Acros Organics, Brockmann I, 50-200 μm particle size, load minimal PhMe, elute 30-50-100% ether in hexanes).

Note 1: Ketone 10 is unstable and should be used immediately for the next reaction. A yellow/brown coloration and enone impurities (from ether ring fragmentation) are observed on prolonged standing under ambient conditions, which is accelerated in the presence of acids/bases and/or proton sources.

¹H NMR (600 MHZ, CDCl₃) δ 2.89 (ddd, J=11.2, 3.7, 2.5 Hz, 1H), 2.68 (dqd, J=11.1, 6.2, 2.5 Hz, 1H), 2.64 (d, J=19.3 Hz, 1H), 2.35 (bs, 1H), 2.18 (d, J=3.5 Hz, 1H), 2.02-1.94 (m, 3H), 1.94 (dd, J=19.3, 1.5 Hz, 1H), 1.83-1.59 (m, 7H), 1.57-1.46 (m, 2H), 1.45-1.14 (m, 5H), 1.28 (s, 3H), 1.14-0.99 (m, 2H), 1.08 (d, J=6.2 Hz, 3H).

¹³C NMR (151 MHz, CDCl₃) δ 213.4, 86.3, 74.6, 61.2, 56.5, 52.8, 52.4, 49.6, 43.1, 42.4, 36.0, 35.2, 34.1, 32.4, 30.5, 25.7, 25.4, 25.4, 23.4, 23.1.

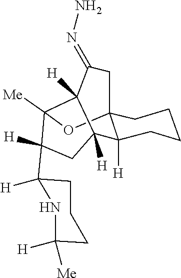 [SMILES: CC1CCCC([C@H]2C[C@H]3C4CCCCC45C/C(=N\N)[C@H]3C2(C)O5)N1]

(2R,6R)-2-((2R,3aS,5aS,9aS,9bS)-4-hydrazineylidene-3-methyldodecahydro-3,5a-epoxycyclopenta[a]naphthalen-2-yl)-6-methylpiperidine (SI-8)

[H][C@]1([C@@]2([H])CCC[C@@]([H])(C)N2[H])C[C@@]3([H])[C@@]4([H])[C@]5(CCCC4)C/C([C@@]3([H])C(C)1O5)=N\N

### Hydrazone Condensation.

In a 1-dram vial, a solution of crude ketone 10 from the previous reaction (assume 0.196 mmol, 1 equiv.) in EtOH (2 mL) was added hydrazine hydrate (97 μL, ˜2 mmol, ˜ 20 equiv.). The vial was sealed and the reaction was heated with stirring at 50° C. for 12 h upon which LCMS analysis showed negligible starting material (M+H, m/z=+318) remaining. The reaction was concentrated under nitrogen flow, azeotroping with toluene at least 3 times, then placed under high vacuum. The residue was used directly for the next reaction. Alternatively, purified ketone 10 could be subjected to the same procedure to afford hydrazone SI-8 in purity sufficient for characterization, contaminated by toluene and BHT (see note 1).

Note 1: hydrazone is formed in high diastereomeric purity (¹H/¹³C/2D data show mostly one diastereomer present; unclear if residual peaks (<5% level) belong to other diastereomer or unassigned byproducts). Attempted NOESY was inconclusive regarding hydrazone stereochemistry but gave strong EXSY correlations between the hydrazone NH₂ and residual PhMe CH₃.

¹H NMR (600 MHZ, CDCl₃) δ 4.94 (bs, 2H), 2.86 (ddd, J=11.2, 3.4, 2.5 Hz, 1H), 2.67 (dqd, J=11.1, 6.2, 2.6 Hz, 1H), 2.62 (d, J=17.9 Hz, 1H), 2.15 (d, J=4.1 Hz, 1H), 1.94 (td, J=12.1, 5.8 Hz, 1H), 1.88-1.84 (m, 1H), 1.84-1.65 (m, 7H), 1.65-1.49 (m, 4H), 1.44-1.35 (m, 3H), 1.35-1.14 (m, 3H), 1.24 (s, 3H), 1.12-0.99 (m, 2H), 1.07 (d, J=6.2 Hz, 3H).

¹³C NMR (151 MHz, CDCl₃) δ 152.4, 86.0, 72.7, 57.0, 53.0, 52.4, 52.3, 50.0, 42.0, 36.5, 34.5, 34.1, 32.3, 30.5, 29.9, 25.7, 25.4, 25.1, 23.4, 23.2.

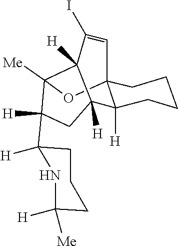 [SMILES: CC1CCCC([C@H]2C[C@H]3C4CCCCC45C=C(I)[C@H]3C2(C)O5)N1]

(2R,6R)-2-((2R,3aR,5aR,9aS,9bS)-4-iodo-3-methyl-1,2,3,3a,6,7,8,9,9a,9b-decahydro-3,5a-epoxycyclopenta[a]naphthalen-2-yl)-6-methylpiperidine (SI-9)

[H][C@]1([C@@]2([H])CCC[C@@]([H])(C)N2[H])C[C@@]3([H])[C@@]4([H])[C@]5(CCCC4)C═C(I)[C@@]3([H])C(C)1O5

### Hydrazone Iodination.

Crude hydrazone SI-8 from the previous reaction (assume 0.196 mmol, 1 equiv.) was taken up in dry tetrahydrofuran (4 mL) under argon, and dry Et₃N (139 μL, 1 mmol, 5 equiv.) was added. A solution of iodine in ether (0.2 M, 2.5 mL, 2.5 equiv.) was added dropwise to the stirred reaction over 10 min, which went from colorless to yellow to persistent orange-brown as more iodine solution was added. Stirring was continued for an additional 10-15 min, after which the reaction was quenched with a mixture of saturated aqueous Na₂SO₃ (4 mL) and saturated aqueous NaHCO₃ (4 mL). The mixture was extracted with ethyl acetate (4×10 mL), and the combined organic extracts were dried over Na₂SO₄ and concentrated under reduced pressure to afford a yellow residue (90 mg). NMR analysis (C₆D₆) indicated the presence of a >6:1 mixture of vinyl iodides, integrating the peaks & 6.66 (d, J=1.8 Hz, 1H, vinyl H (undesired)) and δ 6.60 (d, J=1.8 Hz, 1H, vinyl H (desired)). The residue was taken immediately into the next reaction, or subjected to column chromatography (basic alumina, Acros Organics, Brockmann I, 50-200 μm particle size, load minimal PhMe, elute hexane to 2-3% EtOAc/hexanes) to afford pure material.

¹H NMR (600 MHZ, CDCl₃) δ 6.75 (d, J=1.9 Hz, 1H), 2.81 (ddd, J=11.2, 3.6, 2.5 Hz, 1H), 2.71 (dd, J=4.1, 1.9 Hz, 1H), 2.64 (dqd, J=11.0, 6.2, 2.5 Hz, 1H), 1.94 (dbt, J=12.3, 2.8 Hz, 1H), 1.89 (td, J=12.2, 6.0 Hz, 1H), 1.80-1.69 (m, 4H), 1.69-1.64 (m, 1H), 1.61-1.55 (m, 1H), 1.55-1.49 (m, 2H), 1.49-1.30 (m, 4H), 1.29-1.11 (m, 4H), 1.18 (s, 3H), 1.09-0.95 (m, 1H), 1.03 (d, J=6.2 Hz, 3H), 0.84 (qd, J=13.0, 3.7 Hz, 1H).

¹³C NMR (151 MHZ, CDCl₃) δ ¹³C NMR (151 MHZ, CDCl₃) δ 144.7, 91.3, 85.3, 76.5, 62.0, 56.9, 52.4, 52.3, 51.5, 40.1, 34.1, 34.0, 32.2, 32.2, 31.8, 25.5, 25.4, 23.6, 23.5, 23.4.

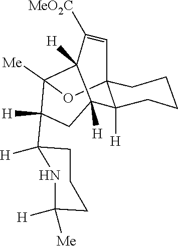 [SMILES: COC(=O)C1=CC23CCCCC2[C@@H]2C[C@H](C4CCCC(C)N4)C(C)(O3)[C@H]12]

methyl (2R,3aS,5aR,9aS,9bS)-3-methyl-2-((2R,6R)-6-methylpiperidin-2-yl)-1,2,3,3a,6,7,8,9,9a,9b-decahydro-3,5a-epoxycyclopenta[a]naphthalene-4-carboxylate (1)

[CO2Me]C1-C[C@@]2(CCCC3)[C@]3([H])[C@@]4([H])[C@]1([H])C([C@@]([H])([C@@]5([H])CCC[C@@]([H])(C)N5[H])C4)(C)O2

### Pd-Catalyzed Carbonylation and Purification of GB18.

Crude iodide SI-9 (assume 0.196 mmol, 1 equiv.) from the previous reaction was transferred into a flame-dried culture tube (13×100 mm) with minimal CH₂Cl₂, evaporated to dryness, and azeotroped with PhMe (3×) by rotary evaporation then high vacuum. A stir bar was added and the reaction tube charged with Pd(PPh₃)₂Cl₂ (Aldrich, 99.99% trace metals basis, 1.4 mg, 0.002 mmol, 0.01 equiv.). In a well-ventilated fume hood, the reaction vessel was evacuated (via a vacuum line setup where the pump exhaust was vented into a well-ventilated fume hood) then backfilled with a balloon of CO gas (3×). Anhydrous DMF (Acros Organics, stored over 4 Å molecular sieves, 400 μL, presparged with CO gas for at least 15 min prior to use) and anhydrous MeOH (Acros Organics, 400 μL, presparged with CO gas for at least 15 min prior to use) were added to the reaction vessel, followed by Et₃N (279 μL, 2 mmol, 10 equiv.). The reaction was placed in a 42° C. sand bath for 19 h upon which an approximate uncalibrated ion count ratio of 100:1 product (M+H, m/z=+360) to starting material (M+H, m/z=+428) was observed by LCMS analysis. The reaction was cooled to room temperature, diluted with EtOAc, and filtered through celite. The filtrate was blown down under nitrogen flow to a reddish-brown residue. NMR analysis of the crude (C₆D₆) was consistent with a dr between 5-6:1. Column chromatography (basic alumina, Acros Organics, Brockmann I, 50-200 μm particle size, load minimal PhMe, elute 50% ether/hexanes) afforded mostly pure GB18 (25 mg, 35%, ˜30% adjusted for purity, major contaminant BHT). Preparative TLC (SiO₂, eluted with quarter-saturated NH₃ (g) in CH₂Cl₂) afforded pure GB18, which could be separated into its enantiomers by preparative SFC. A single crystal of slow-moving enantiomer ent-1 was grown by slow evaporation of a solution in methanol and subjected to X-ray analysis (Cu K_{α} radiation (λ=1.54178)). Absolute stereochemistry (verified by a Flack parameter=−0.00 (6)) was determined to be that shown in FIG. 2, correlating this slow-moving enantiomer ent-1 with ent-GB18, the antipode of natural isolate (crystal structure CCDC793595). Fast-moving enantiomer nat-1 was therefore assigned as the natural enantiomer, nat-GB18.

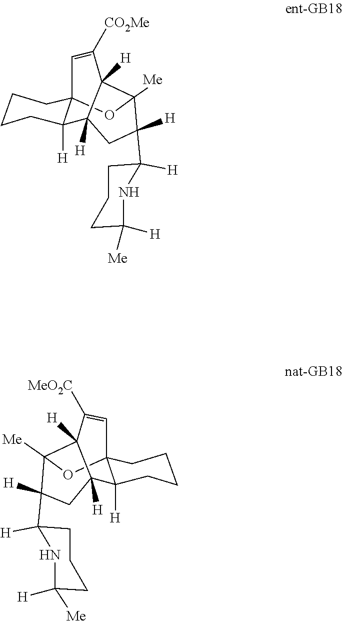 [SMILES: COC(=O)C1=CC23CCCCC2[C@H]2C[C@@H](C4CCCC(C)N4)C(C)(O3)[C@@H]12 ; COC(=O)C1=CC23CCCCC2[C@@H]2C[C@H](C4CCCC(C)N4)C(C)(O3)[C@H]12]

Structures of ent- and nat-GB18 with absolute stereochemistry are shown above.

¹H NMR (600 MHZ, CDCl₃) δ 7.19 (d, J=1.7 Hz, 1H), 3.79 (s, 3H), 2.97 (dd, J=4.2, 1.7 Hz, 1H), 2.83 (dbt, J=11.2, 2.6 Hz, 1H), 2.65 (dqd, J=11.1, 6.2, 2.5 Hz, 1H), 2.02 (dbt, J=12.3, 2.9 Hz, 1H), 1.97 (td, J=11.4, 5.9 Hz, 1H), 1.85-1.72 (m, 4H), 1.72-1.55 (bs, 1H), 1.62-1.55 (m, 2H), 1.55-1.46 (m, 4H), 1.46-1.40 (dm, J=13.1 Hz, 1H), 1.40-1.29 (m, 2H), 1.24-1.14 (m, 2H), 1.08-0.97 (m, 1H), 1.05 (d, J=6.2 Hz, 3H), 0.99 (s, 3H), 0.70 (qd, J=13.2, 3.6 Hz, 1H).

¹³C NMR (151 MHz, CDCl₃) δ 165.9, 144.7, 132.7, 84.8, 73.4, 57.2, 52.4, 52.2, 52.0, 51.7, 48.2, 39.5, 34.1, 33.8, 32.6, 32.29, 32.26, 25.53, 25.45, 23.9, 23.6, 23.4.

### Additional Example Syntheses:

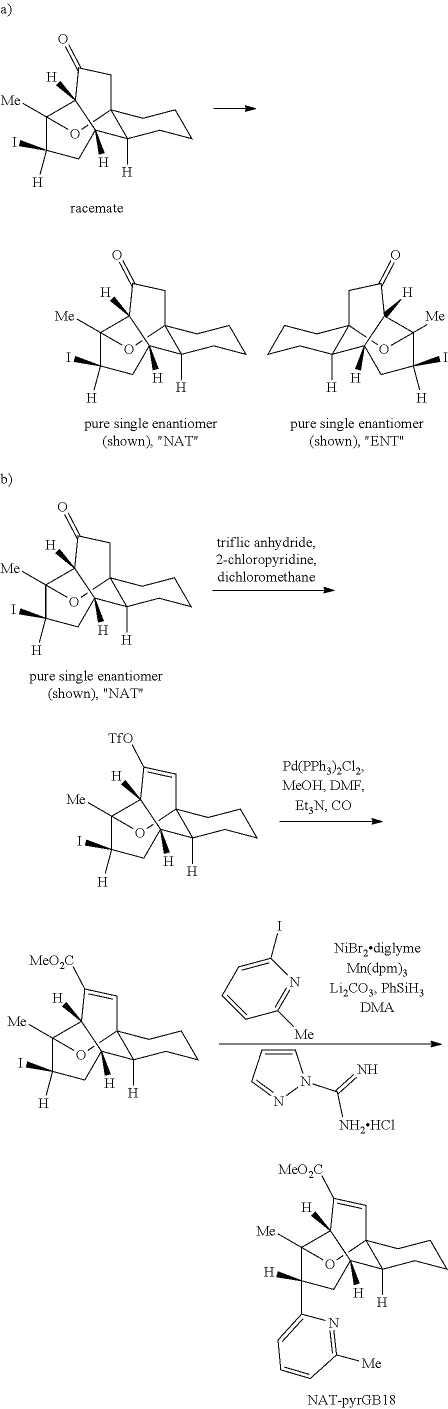

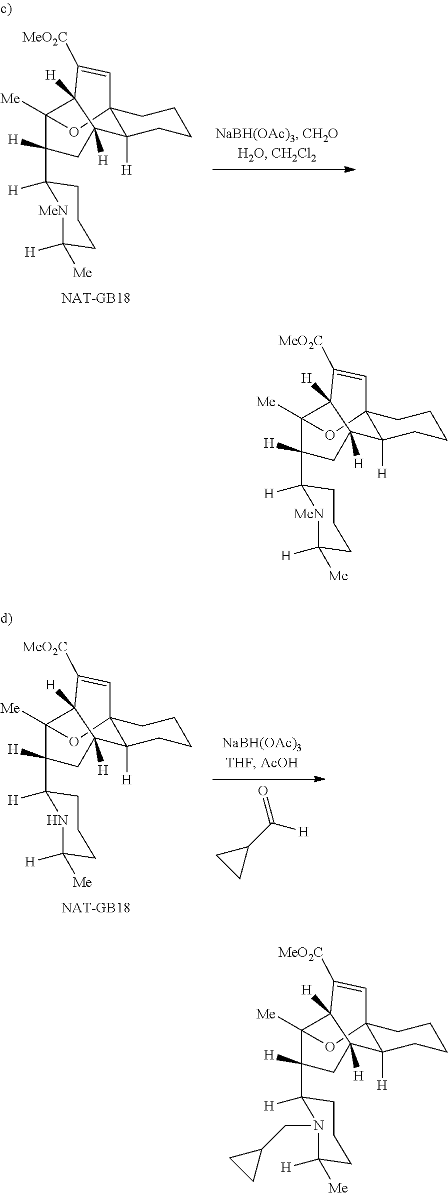 [SMILES: COC(=O)C1=CC23CCCCC2[C@@H]2C[C@H](C4CCCC(C)N4C)C(C)(O3)[C@H]12 ; COC(=O)C1=CC23CCCCC2[C@@H]2C[C@H](C4CCCC(C)N4C)C(C)(O3)[C@H]12 ; COC(=O)C1=CC23CCCCC2[C@@H]2C[C@H](C4CCCC(C)N4)C(C)(O3)[C@H]12 ; O=CC1CC1 ; COC(=O)C1=CC23CCCCC2[C@@H]2C[C@H](C4CCCC(C)N4CC4CC4)C(C)(O3)[C@H]12]

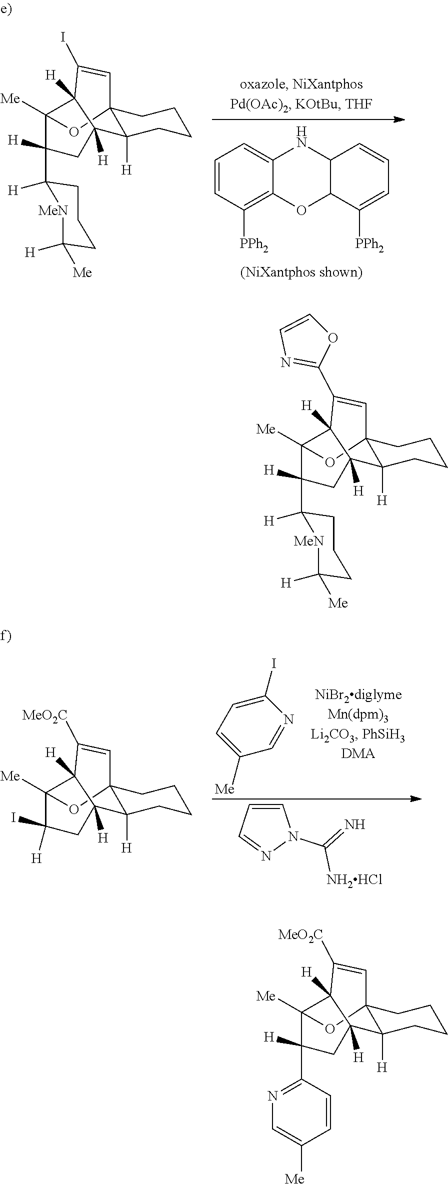

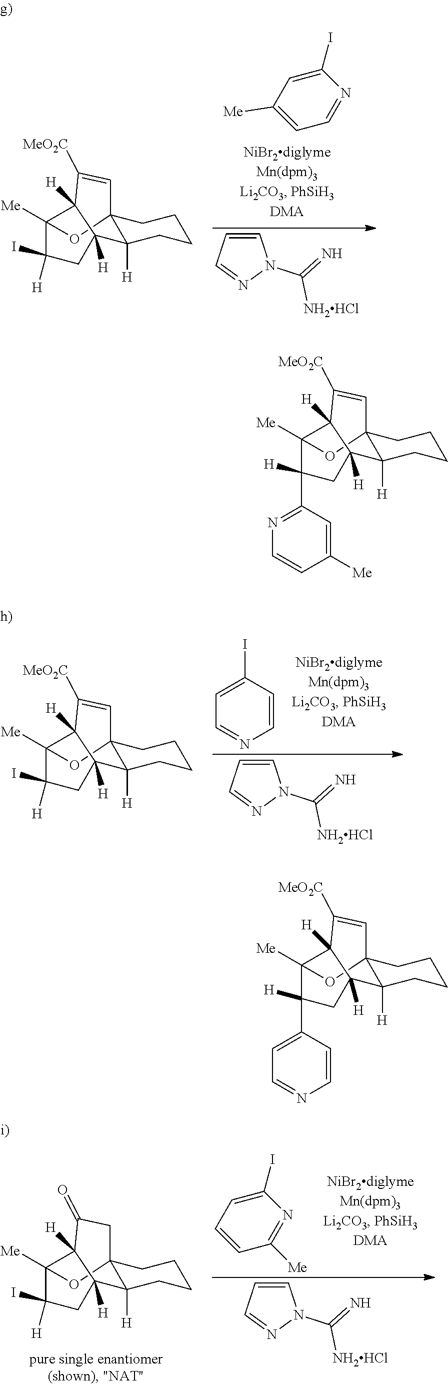

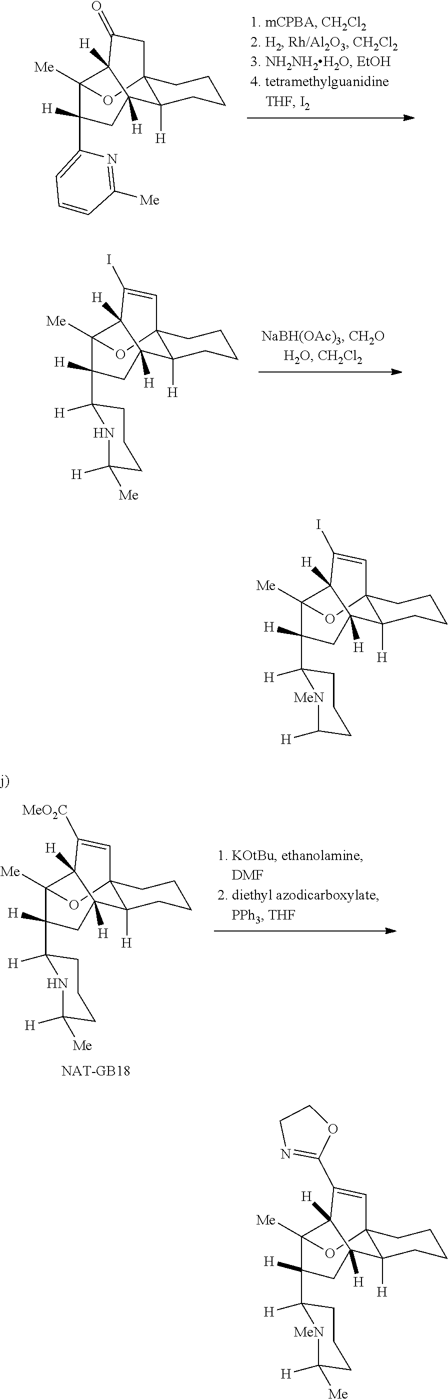 [SMILES: Cc1cccc([C@H]2C[C@H]3C4CCCCC45CC(=O)[C@H]3C2(C)O5)n1 ; CC1CCCC([C@H]2C[C@H]3C4CCCCC45C=C(I)[C@H]3C2(C)O5)N1 ; CN1CCCCC1[C@H]1C[C@H]2C3CCCCC34C=C(I)[C@H]2C1(C)O4 ; COC(=O)C1=CC23CCCCC2[C@@H]2C[C@H](C4CCCC(C)N4)C(C)(O3)[C@H]12 ; CC1CCCC([C@H]2C[C@H]3C4CCCCC45C=C(C4=NCCO4)[C@H]3C2(C)O5)N1C]

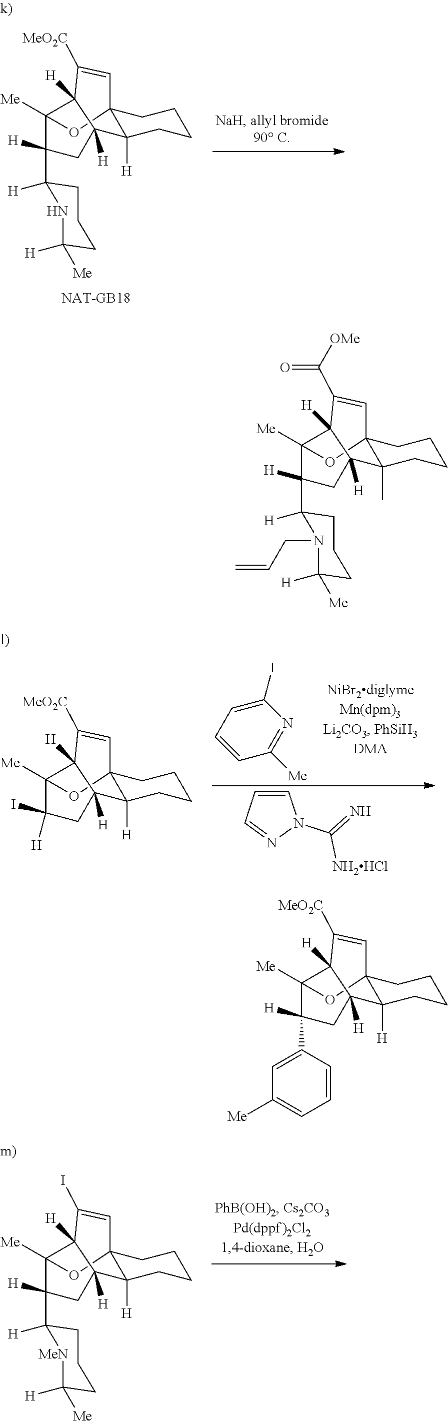

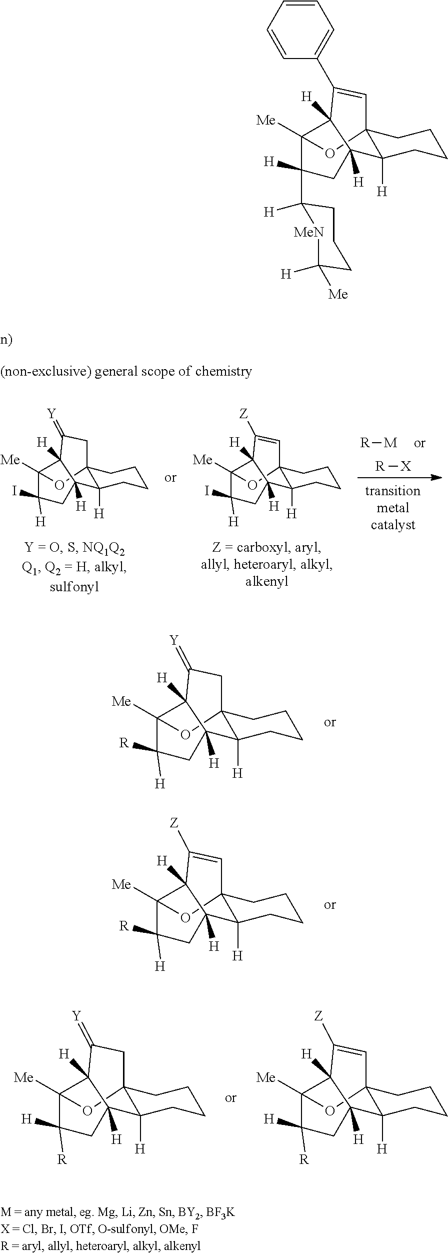

### KOR and MOR ³⁵S-GTPgS Binding:

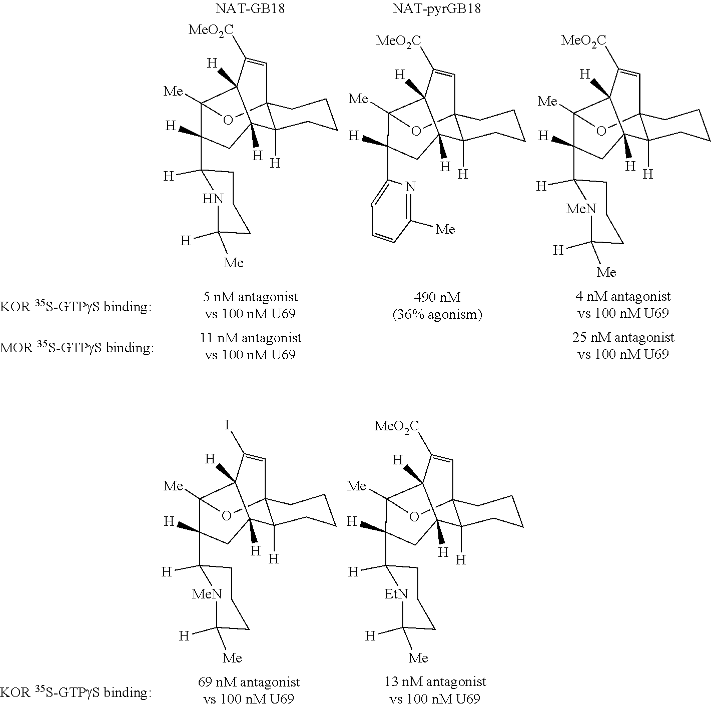

### KOR CAMP Activity

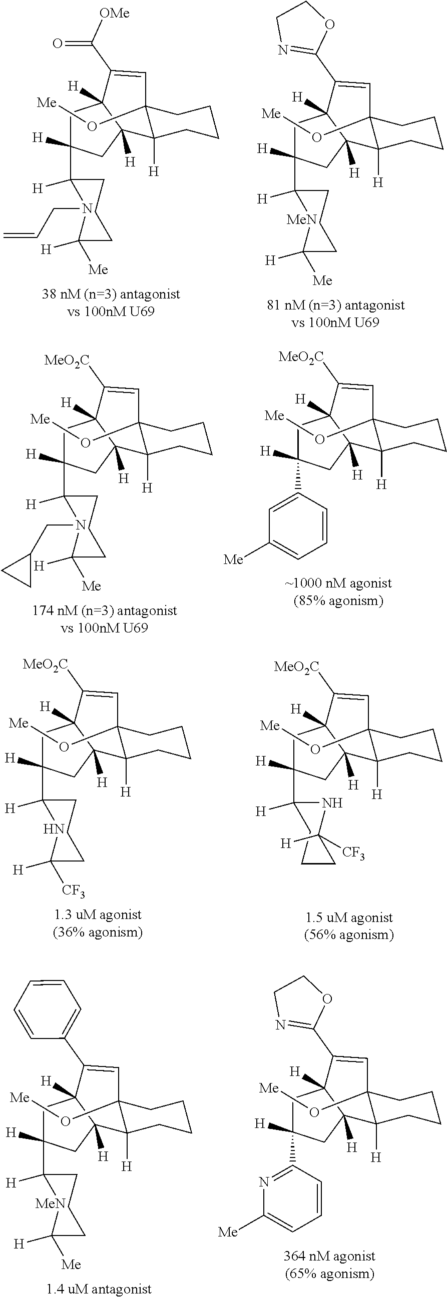

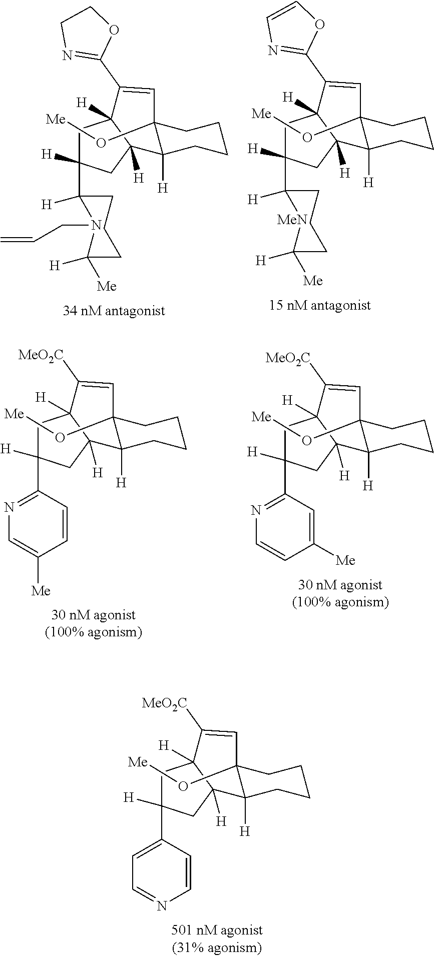

### Bioassay Protocols

KOR assay Protocols

### GTPγS Coupling

Homogenization buffer: 10 mM Tris pH 7.4, 1 mM EDTA, 100 mM NaCl.
Assay buffer: 50 mM Tris pH 7.4, 5 mM MgCl₂, 1 mM EDTA, 100 mM NaCl.
Final per well: 200 μL, 15 μg protein, 3 μM GDP, 70,000 cpm, 1% DMSO.
Incubate at RT, 1 hr.
Filter with cold water, punch filter paper into new plate, let dry at RT, ON.
Add 50 μL scintillation fluid, read on counter.
   βarrestin2 Recruitment
Dilute to 2.5E5 cells/mL in 1% FBS Opti-MEM and plate 20 μL/well. Incubate 37° C., 18 hrs.
Add drugs and incubate at 37° C., 90 min.
Final per well: 25 μL, 5,000 cells, 1% DMSO.
Add DRX substrate reagents and incubate at RT, dark, 60 min.
Read luminescence at 1000-ms integration.

### CAMP Inhibition

Dilute to 8E5 cells/mL in 1% FBS Opti-MEM and plate 5 μL/well. Incubate 37° C., 3 hrs.
Add drugs and incubate at 37° C., 30 min.
Final per well: 10 μL, 4,000 cells, 20 μM FSK, 25 μM PDE-IV, 1% DMSO.
Add HTRF reagents and incubate at RT, dark, 60 min.
Read fluorescence at 340/620 and 340/665 nm.

### MOR Assay Protocols

GTPγS coupling
Homogenization buffer: 10 mM Tris pH 7.4, 1 mM EDTA, 100 mM NaCl.
Assay buffer: 50 mM Tris pH 7.4, 5 mM MgCl₂, 1 mM EDTA, 100 mM NaCl.
Final per well: 200 μL, 10 μg protein, 50 μM GDP, 70,000 cpm, 1% DMSO.
Incubate at RT, 1 hr.
Filter with cold water, punch filter paper into new plate, let dry at RT, ON.
Add 50 μL scintillation fluid, read on counter.
   βarrestin2 Recruitment
Dilute to 2.5E5 cells/mL in 1% FBS Opti-MEM and plate 20 μL/well. Incubate 37° C., 18 hrs.
Add drugs and incubate at 37° C., 90 min.
Final per well: 25 μL, 5,000 cells, 1% DMSO.
Add DRX substrate reagents and incubate at RT, dark, 60 min.
Read luminescence at 1000-ms integration.

### CAMP Inhibition

Dilute to 8E5 cells/mL in 1% FBS Opti-MEM and plate 5 μL/well. Incubate 37° C., 3 hrs.
Add drugs and incubate at 37° C., 30 min.
Final per well: 10 μL, 4,000 cells, 20 μM FSK, 25 μM PDE-IV, 1% DMSO.
Add HTRF reagents and incubate at RT, dark, 60 min.
Read fluorescence at 340/620 and 340/665 nm.
   the Following Tables Show KOR cAMP Screenings with GB18 Analogs

| | | Agonist | | | Antagonist | |
|---|---|---|---|---|---|---|
| | | EC₅₀ | | Eₘₐₓ | vs. 100 nM U69,593 | |
| Name | Structure | (nM) | 95% Cl | (% U69,593) | IC₅₀ (nM) | 95% Cl |
| U69,593 | | 2.2 | 1.7-3.0 | 100 | | |
| NorBNl | | — | — | — | 1.4 | 1.0-2.0 |
| | 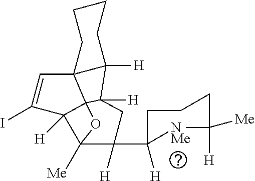 [SMILES: CC1CCCC(C2CC3C4CCCCC45C=C(I)C4C2(C)OC345)N1C] | nc (n = 1) | — | — | 830 (n = 1) | 158-4163 |
| | 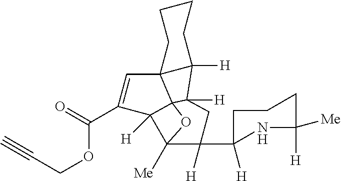 [SMILES: C#CCOC(=O)C1=CC23CCCCC2C2CC(C4CCCC(C)N4)C4(C)OC23C14] | nc (n = 1) | — | 5* | 556 (n = 1) | poor fit |
| | 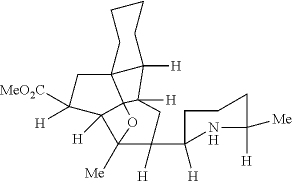 [SMILES: COC(=O)C1CC23CCCCC2C2CC(C4CCCC(C)N4)C4(C)OC23C14] | nc (n = 1) | — | 13* | 4997 (n = 1) | poor fit |
| | 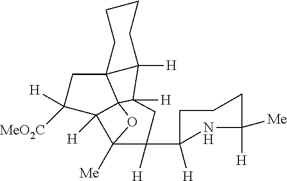 [SMILES: COC(=O)C1CC23CCCCC2C2CC(C4CCCC(C)N4)C4(C)OC23C14] | nc (n = 1) | — | 10* | >10,000 (n = 1) | poor fit |
| | 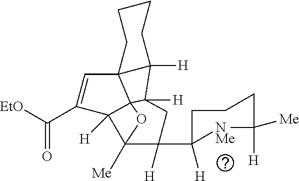 [SMILES: CCOC(=O)C1=CC23CCCCC2C2CC(C4CCCC(C)N4C)C4(C)OC23C14] | nc (n = 1) | — | 15* | 3393 (n = 1) | 1477-9302 |
| | 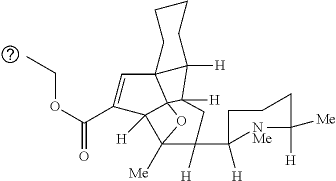 [SMILES: CCOC(=O)C1=CC23CCCCC2C2CC(C4CCCC(C)N4C)C4(C)OC23C14] | nc (n = 1) | — | 14* | nc (n = 1) | — |
| | 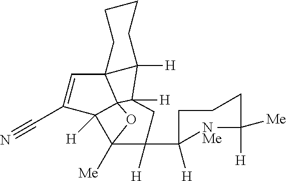 [SMILES: CC1CCCC(C2CC3C4CCCCC45C=C(C#N)C4C2(C)OC345)N1C] | nc (n = 1) | — | 8* | 27 [symbol] 0 (n = 1) | poor fit |
| | 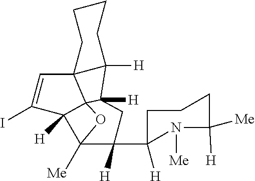 [SMILES: CC1CCCC([C@H]2C[C@H]3C4CCCCC45C=C(I)[C@H]4C2(C)OC345)N1C] | not tested | — | — | 1400 (n = 1) | 687-2981 |

[symbol]

indicates data missing or illegible when filed

| | | Agonist | | | Antagonist | |
|---|---|---|---|---|---|---|
| | | EC₅₀ | | Eₘₐₓ | vs. 100 nM U69,593 | |
| Name | Structure | (nM) | 95% Cl | (% U69,593) | IC₅₀ (nM) | 95% Cl |
| U69,593 | | 2.2 | 1.7-3.0 | 100 | | |
| NorBNl | | — | — | — | 1.4 | 1.0-2.0 |
| | 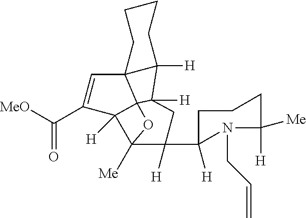 [SMILES: C=CCN1C(C)CCCC1C1CC2C3CCCCC34C=C(C(=O)OC)C3C1(C)OC234] | nc (n = 1) | — | 2* | 3 [symbol] (n = 3) | 21-72 |
| | 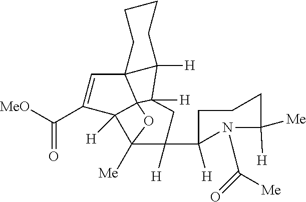 [SMILES: COC(=O)C1=CC23CCCCC2C2CC(C4CCCC(C)N4C(C)=O)C4(C)OC23C14] | nc (n = 1) | — | 10* | >10,000 (n = 1) | — |
| | 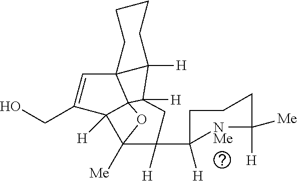 [SMILES: CC1CCCC(C2CC3C4CCCCC45C=C(CO)C4C2(C)OC345)N1C] | nc (n = 1) | — | 12* | 116 (n = 1) | poor fit |
| | 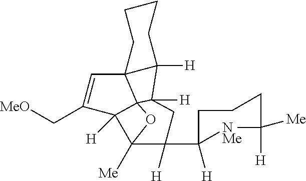 [SMILES: COCC1=CC23CCCCC2C2CC(C4CCCC(C)N4C)C4(C)OC23C14] | nc (n = 1) | — | 14* | 3220 (n = 1) | poor fit |
| | 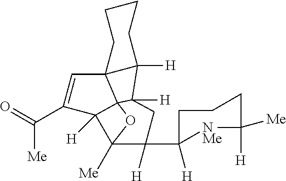 [SMILES: CC(=O)C1=CC23CCCCC2C2CC(C4CCCC(C)N4C)C4(C)OC23C14] | nc (n = 1) | — | 12* | [symbol] [symbol] 2 (n = 2) | 421-1899 |
| | 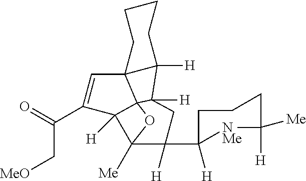 [SMILES: COCC(=O)C1=CC23CCCCC2C2CC(C4CCCC(C)N4C)C4(C)OC23C14] | nc (n = 1) | — | 10* | nc (n = 1) | — |
| | 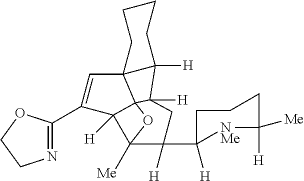 [SMILES: CC1CCCC(C2CC3C4CCCCC45C=C(C4=NCCO4)C4C2(C)OC345)N1C] | nc (n = 1) | — | 4* | [symbol] 1 (n = 3) | 42-153 |

[symbol]

indicates data missing or illegible when filed

| | | Agonist | | | Antagonist | |
|---|---|---|---|---|---|---|
| | | EC₅₀ | | Eₘₐₓ | vs. 100 nM U69,593 | |
| Name | Structure | (nM) | 95% Cl | (% U69,593) | IC₅₀ (nM) | 95% Cl |
| U69,593 | | 2.2 | 1.7-3.0 | 100 | | |
| NorBNl | | — | — | — | 1.4 | 1.0-2.0 |
| | 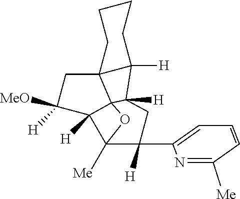 | nc (n = 1) | — | 5* | nc (n = 1) | — |
| | 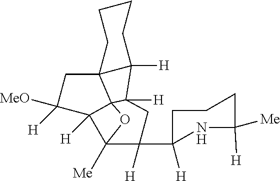 [SMILES: COC1CC23CCCCC2C2CC(C4CCCC(C)N4)C4(C)OC23C14] | nc (n = 1) | — | 10* | 6246 (n = 1) | poor fit |
| | 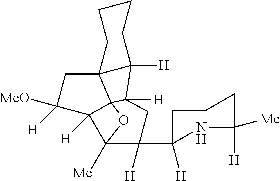 [SMILES: COC1CC23CCCCC2C2CC(C4CCCC(C)N4)C4(C)OC23C14] | nc (n = 1) | — | 17* | nc (n = 1) | — |
| | 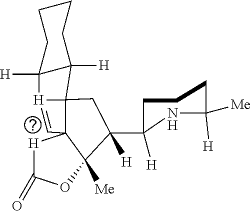 | nc (n = 1) | — | +z899 * | nc (n = 1) | — |
| | 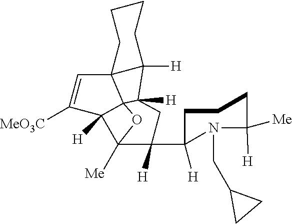 | nc (n = 1) | — | 8* | 174 (n = 3) | 96-311 |
| | 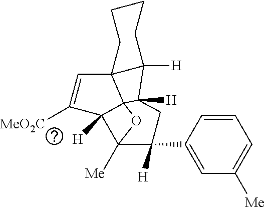 [SMILES: COC(=O)C1=CC23CCCCC2[C@@H]2C[C@H](c4cccc(C)c4)C4(C)OC23[C@@H]14] | 1,050 (n = 3) | 306-3823 | 60* | nc (n = 1) | — |
| | 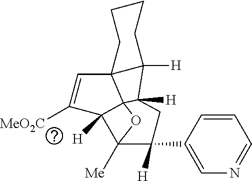 [SMILES: COC(=O)C1=CC23CCCCC2[C@@H]2C[C@H](c4cccnc4)C4(C)OC23[C@@H]14] | nc (n = 2) | — | 19* | >10,000 (n = 1) | — |

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various publications (e.g., issued patents, published patent applications, journal articles, and other publications), each of which are incorporated herein by reference.

What is claimed is:

1. A process for preparing a scalemic or racemic mixture of Galbulimima alkaloid GB18, comprising a mixture of the enantiomers of Formulae (1) and (2): 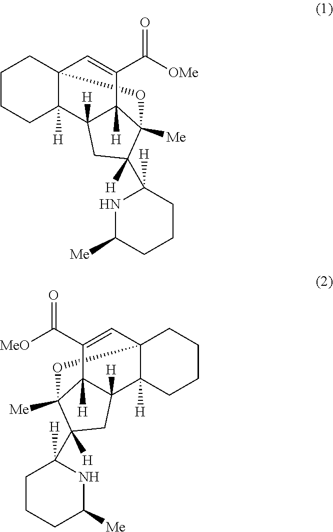 [SMILES: COC(=O)C1=C[C@]23CCCC[C@@H]2[C@H]2C[C@@H]([C@H]4CCC[C@@H](C)N4)[C@@](C)(O3)[C@@H]12 ; COC(=O)C1=C[C@@]23CCCC[C@H]2[C@@H]2C[C@H]([C@@H]4CCC[C@H](C)N4)[C@](C)(O3)[C@H]12]
the process comprising the sequential steps of:
(a) epoxidizing the compound of Formula (5) 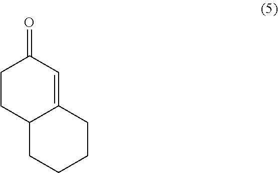 [SMILES: O=C1C=C2CCCCC2CC1]
to form the compound of Formula (SI-2) 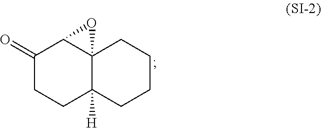 [SMILES: O=C1CC[C@@H]2CCCC[C@@]23O[C@@H]13]
(b) hydrogenating the compound of Formula (SI-2) to form the compound of Formula (6) 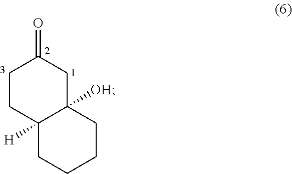 [SMILES: O=C1CC[C@@H]2CCCC[C@]2(O)C1]
(c) silylating the compound of Formula (6) to form the compound of Formula (SI-3) 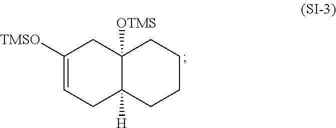
(d) conducting a Saegusa oxidation of the compound of Formula (SI-3) to form the compound of Formula (4) 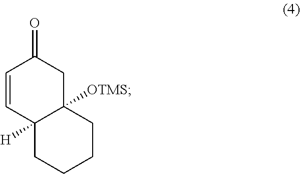
(e) reacting the compound of Formula (4) with the compound of Formula (SI-4) 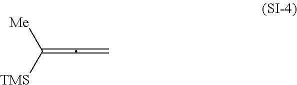
to form the compound of Formula (7) 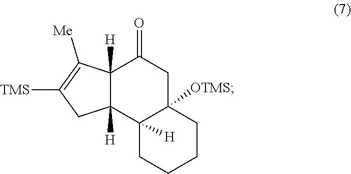
(f) desilylating the compound of Formula (7) to form the compound of Formula (8) 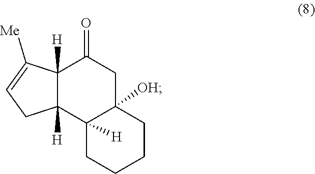
(g) iodoetherifying the compound of Formula (8) to form the compound of Formula (3) 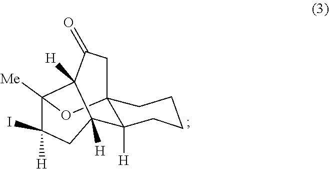 [SMILES: CC12OC34CCCCC3[C@H](C[C@H]1I)[C@H]2C(=O)C4]
(h) conducting a cross-electrophile coupling of the compound of Formula (3) with 2-iodo-6-methylpyridine (SI-6) to form the compound of Formula (2) 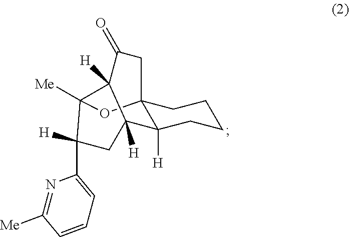
(i) conducting a pyridine N-oxidation of the compound of Formula (2) to form the compound of Formula (9) 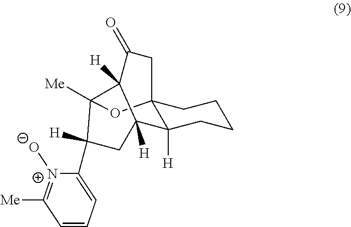
(j) hydrogenating the compound of Formula (9) to form the compound of Formula (10) 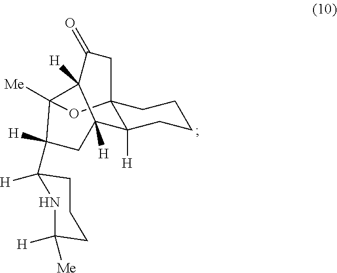 [SMILES: CC1CCCC([C@H]2C[C@H]3C4CCCCC45CC(=O)[C@H]3C2(C)O5)N1]
(k) condensing the compound of Formula (10) with hydrazine/hydrazine hydrate or a hydrazine derivative to form the hydrazone compound of Formula (SI-8) 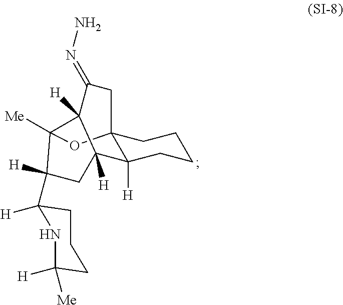 [SMILES: CC1CCCC([C@H]2C[C@H]3C4CCCCC45C/C(=N\N)[C@H]3C2(C)O5)N1]
(l) halogenating the compound of Formula (SI-8) to form the compound of Formula (SI-9) 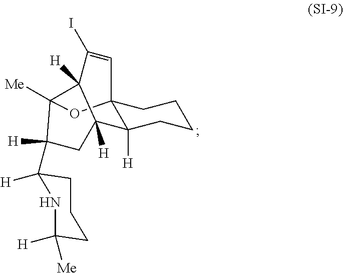 [SMILES: CC1CCCC([C@H]2C[C@H]3C4CCCCC45C=C(I)[C@H]3C2(C)O5)N1]
and
(m) conducting a carbonylation of the compound of Formula (SI-9) to form the scalemic or racemic mixture.

2. The process according to claim 1, wherein in step (a), the epoxidation of the compound of Formula (SI-2) is carried out in the presence of a peroxide, a peroxyacid reagent, or derivatives thereof.

3. The process according to claim 1 or 2, wherein in step (b), the hydrogenation of the compound of Formula (SI-2) is a metal-catalyzed hydrogenation.

4. The process according to claim 3, wherein the metal-catalyzed hydrogenation is conducted in the presence of ingredients comprising Pd/C and hydrogen gas.

5. The process according to claim 4, wherein the ingredients further comprise at least one solvent selected from the group consisting of 1,1,1,3,3,3-hexafluoro-2-propanol (HFIP) and ethyl acetate.

6. The process according to any one of claims 1-5, wherein in step (c), the silylation of the compound of Formula (6) is carried out in the presence of at least one silylating agent selected from the group consisting of trimethylsilyl triflate, a halotrimethyl silane, hexamethyldisilazane, N,O-bis(trimethylsilyl) acetamide, N,O-bis(trimethylsilyl) trifluoroacetamide, and N,O-bis(trimethylsilyl) carbamate.

7. The process according to any one of claims 1-6, wherein in step (d), the Saegusa oxidation is carried out in the presence of a palladium salt and oxygen gas, or in the presence of an organic oxidizing agent.

8. The process according to claim 7, wherein the palladium salt is at least one selected from the group consisting of Pd(OAc)₂, or PdCl₂, and the organic oxidizing agent is 2-iodoxybenzoic acid.

9. The process according to any one of claims 1-8, wherein in step (e), the reaction of the compound of Formula (4) with the compound of Formula (SI-4) is carried out in the presence of a Lewis acid.

10. The process according to claim 9, wherein the Lewis acid is TiCl₄.

11. The process according to any one of claims 1-10, wherein in step (f), the desilylation of the compound of Formula (7) to form the compound of Formula (8) is carried out in the presence of at least one solvent comprising HFIP.

12. The process according to claim 11, further comprising recrystallizing a crude compound of Formula (8) formed from the desilylation of the compound of Formula (7).

13. The process according to any one of claims 1-10, wherein in step (g), the iodoetherification of the compound of Formula (8) is carried out in the presence of an electrophilic iodine reagent.

14. The process according to claim 13, wherein the electrophilic iodine reagent is N-iodosuccinimide.

15. The process according to any of claims 1-14, wherein in step (h), the cross-electrophile coupling of the compound of Formula (3) with 2-iodo-6-methylpyridine (SI-6) is carried out in the presence of a ligand or a salt thereof.

16. The process according to claim 15, wherein the ligand or ligand salt is 1H-pyrazole-1-carboxamidine hydrochloride.

17. The process according to any one of claims 1-12, wherein in step (i), the pyridine N-oxidation of the compound of Formula (2) is carried out in the presence of meta-Chloroperbenzoic acid or methyltrioxorhenium/hydrogen peroxide.

18. The process according to any one of claims 1-13, wherein in step (j), the hydrogenation of the compound of Formula (9) is a metal catalyzed hydrogenation.

19. The process according to claim 18, wherein metal catalyzed hydrogenation is carried out in the presence of Rhodium/Al₂O₃ and hydrogen gas.

20. The process according to any one of claims 1-19, wherein in step (k), the compound of Formula (10) is condensed with hydrazine hydrate.

21. The process according to any one of claims 1-19, wherein in step (1), the halogenation of the compound of Formula (SI-8) is iodination.

22. The process according to claim 21, wherein the iodination is carried out in the presence of a solution of iodine.

23. The process according to any one of claims 1-22, wherein in step (m), the carbonylation is a metal catalyzed carbonylation.

24. The process according to claim 23, wherein the metal catalyzed carbonylation is carried out in the presence of palladium catalyst(s), carbon monoxide gas, and methanol.

25. The process according to claim 24, wherein the palladium catalyst is selected from the group consisting of Pd(OAc)₂/PPh₃, Pd₂dba₃ (dibenzylidene acetone) Pd(PPh₃) 4, and Pd(MeCN)₂Cl₂.

26. The process according to any one of claims 1-25, further comprising resolving the scalemic or racemic mixture of GB18 into the enantiomers of Formulae (1) and (2).

27. A process for preparing a scalemic or racemic mixture of Galbulimima alkaloid GB18, comprising a mixture of the enantiomers of Formulae (1) and (2): 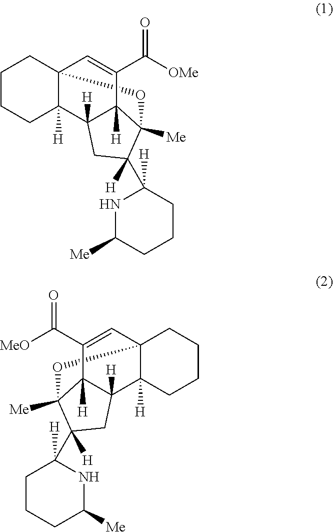 [SMILES: COC(=O)C1=C[C@]23CCCC[C@@H]2[C@H]2C[C@@H]([C@H]4CCC[C@@H](C)N4)[C@@](C)(O3)[C@@H]12 ; COC(=O)C1=C[C@@]23CCCC[C@H]2[C@@H]2C[C@H]([C@@H]4CCC[C@H](C)N4)[C@](C)(O3)[C@H]12]
the process comprising using at least one compound selected from the group consisting of: 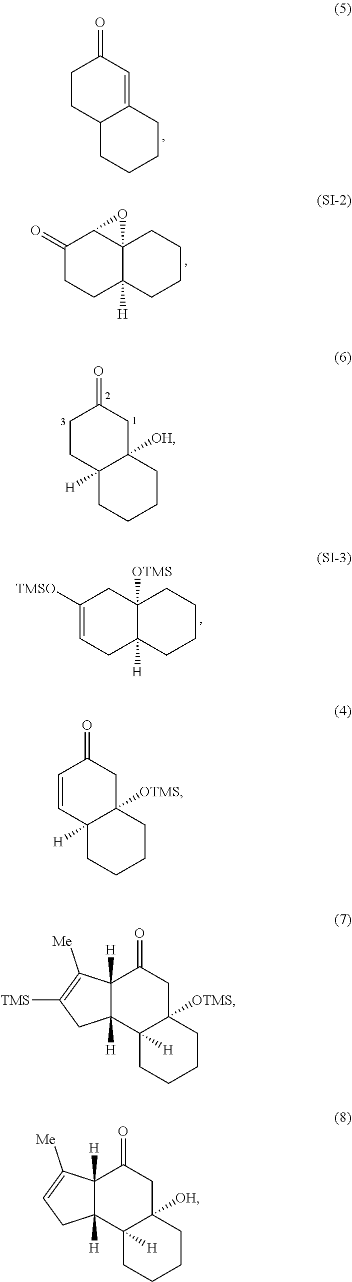 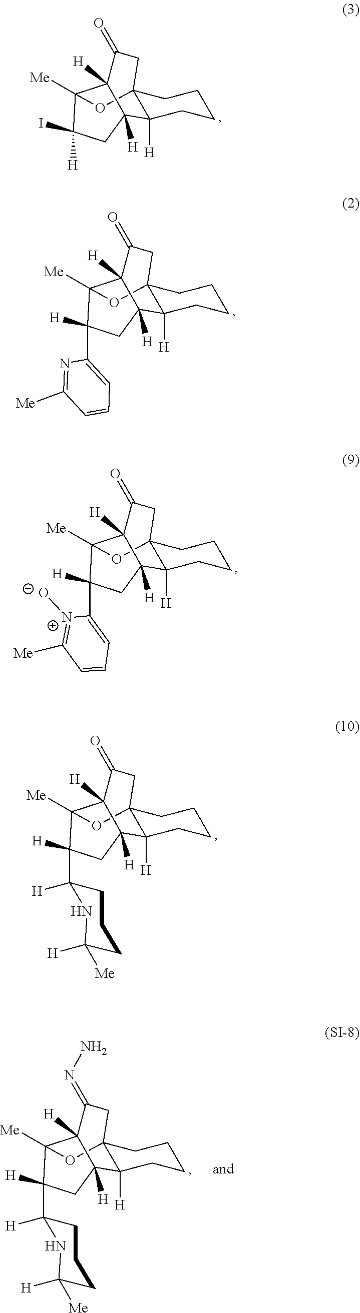 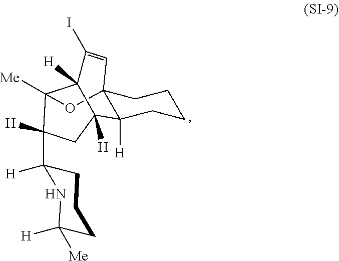
or an enantiomer, a scalemic mixture or a racemic mixture thereof, as an intermediate in the preparation of the scalemic or racemic mixture of Galbulimima alkaloid GB18.

28. The process according to claim 27, further comprising resolving the scalemic or racemic mixture of GB 18 into the enantiomers of Formulae (1) and (2).

29. A compound selected from the group consisting of: 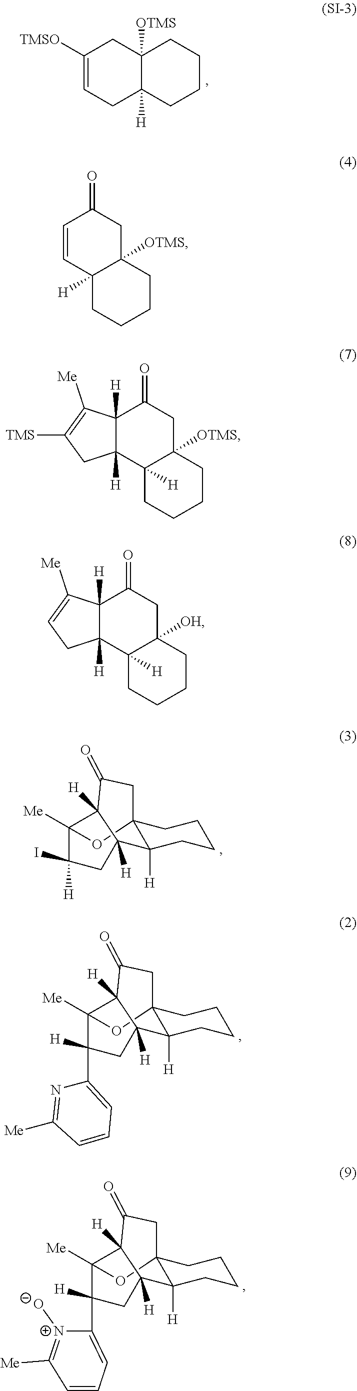 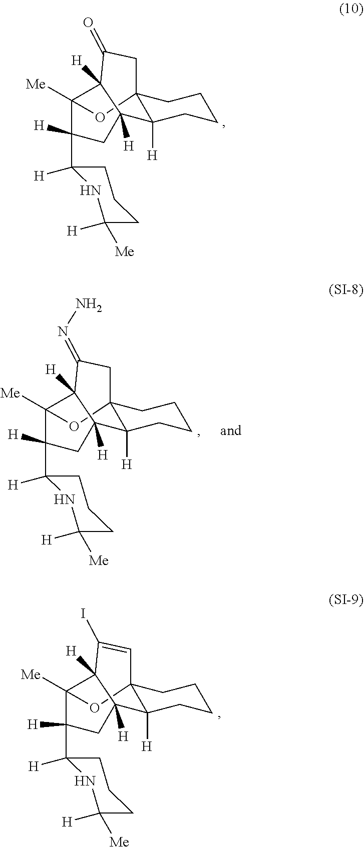 [SMILES: CC1CCCC([C@H]2C[C@H]3C4CCCCC45CC(=O)[C@H]3C2(C)O5)N1 ; CC1CCCC([C@H]2C[C@H]3C4CCCCC45C/C(=N\N)[C@H]3C2(C)O5)N1 ; CC1CCCC([C@H]2C[C@H]3C4CCCCC45C=C(I)[C@H]3C2(C)O5)N1]
or an enantiomer, a scalemic mixture or a racemic mixture thereof.

30. The compound of Formula (1) 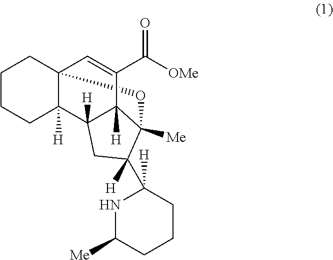 [SMILES: COC(=O)C1=C[C@]23CCCC[C@@H]2[C@H]2C[C@@H]([C@H]4CCC[C@@H](C)N4)[C@@](C)(O3)[C@@H]12]
or a pharmaceutically acceptable salt thereof.

31. A method of antagonizing an opioid receptor in a subject in need of such antagonization, comprising administering to such subject a therapeutically effective amount of a compound of Formula (I) 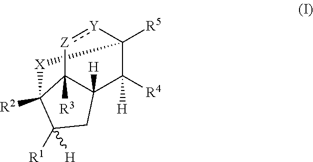
wherein: [symbol]
is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷;
or R⁶ and R⁷ together form monocyclic or bicyclic C₃-C₁₄ cycloalkyl or C₃-C₁₄ heterocycloalkyl, each individually and optionally substituted with one or more R when [symbol] is a single bond;
or R⁶ and R⁷ together form monocyclic or bicyclic C₅-C₁₄ heteroaryl optionally substituted with one or more R when [symbol] is a double bond;
Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇ cycloalkyl), —NMsPh, and —C(═O)OH;
R² and R³ are independently H, C₁-C₆ alkyl, (C₁-C₆ alkyl)-C(═O)—, hydroxy, or —O—(C₁-C₆ alkyl);
or R² and R³ together form C₃-C₇ cycloalkyl or C₃-C₇ heterocycloalkyl;
R⁴ and R⁵ are each independently H or C₁-C₆ alkyl;
or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl); or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
each R^{p} independently is H, C₁-C₆ alkyl, —C(═O)—(C₁-C₆ alkyl), —Si(C₁-C₆ alkyl)₃, —S(═O)₂—(C₁-C₆ alkyl), —S(═O)₂—(C₆-C₁₀ aryl), or —NHTs;
R^{q}, R^{s}, and R^{t} are each independently H or C₁-C₆ alkyl;
wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
or a pharmaceutically acceptable salt thereof.

32. The method according to claim 31, wherein, in Formula (I): [symbol]
is a double bond;
X is O;
Y is CR⁶;
Z is CR⁷;
R¹ is C₆-C₁₀ aryl or C₅-C₁₀ heteroaryl, optionally substituted with 1-3 substituents independently selected from the group consisting of C₁-C₆ alkyl, halo, —OH, —O—C₁-C₆ alkyl, —CN, —NO₂, —CF₃, methylene cyclopropyl, and —C(═O)OH;
R² is C₁-C₆ alkyl;
R³ is H;
R⁴ and R⁵ together with the carbon atoms to which they are shown attached form a C₃-C₇ cycloalkyl, which is optionally substituted with 1-2 substitutents independently selected from the group consisting of C₁-C₆ alkyl, halo, —OH, —O—C₁-C₆ alkyl, —CN, —NO₂, —CF₃, and —C(═O)OH;
R⁶ is H; and
R⁷ is —C(═O)—(C₁-C₆ alkyl).

33. The method according to claim 31 or 32, wherein the compound of Formula (I) or a pharmaceutically acceptable salt thereof is the compound of Formula (2): 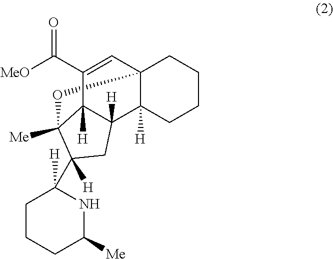 [SMILES: COC(=O)C1=C[C@@]23CCCC[C@H]2[C@@H]2C[C@H]([C@@H]4CCC[C@H](C)N4)[C@](C)(O3)[C@H]12]
or a pharmaceutically acceptable salt thereof.

34. The method according to claim 31, wherein the compound of Formula (I) or a pharmaceutically acceptable salt thereof is selected from the group consisting of: 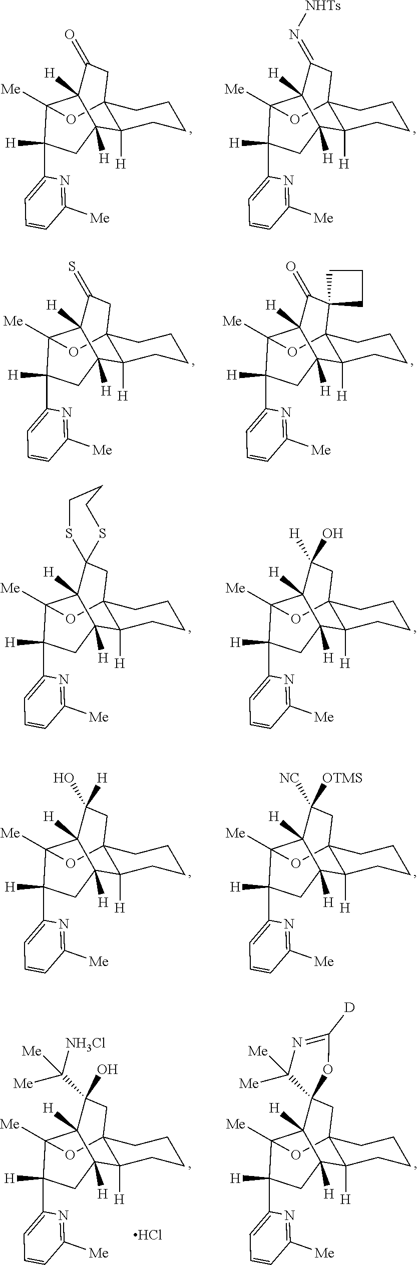 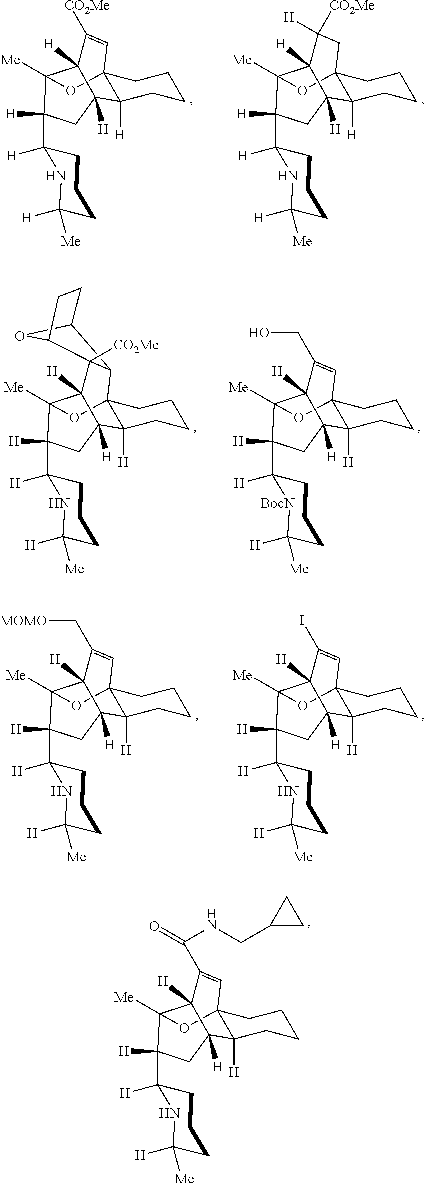 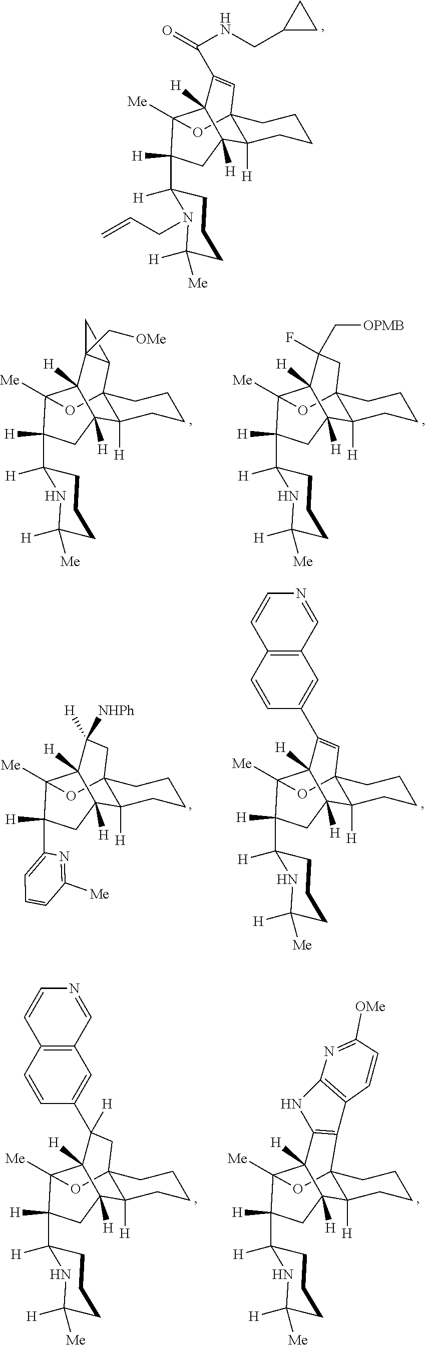 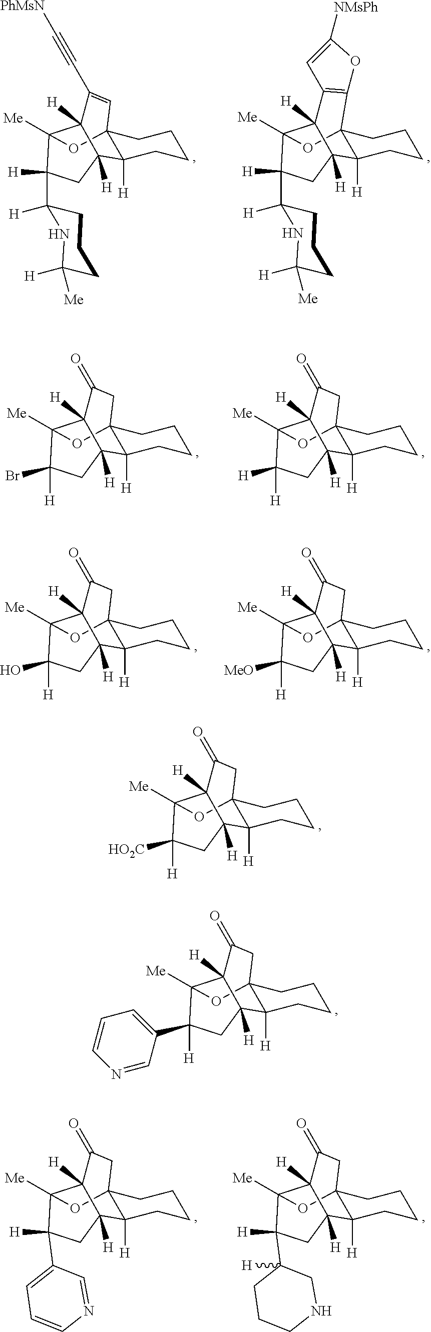 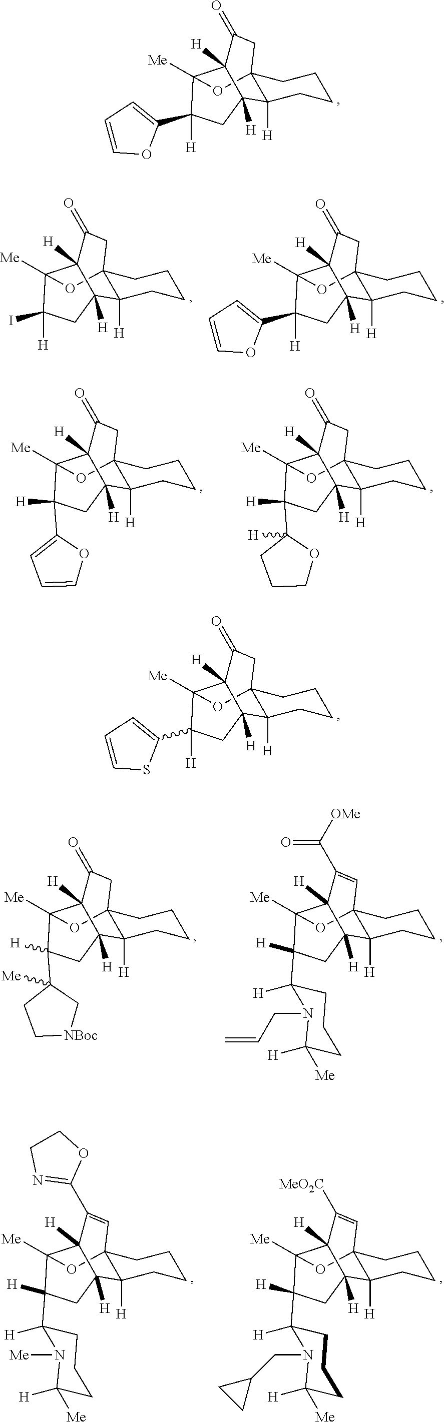 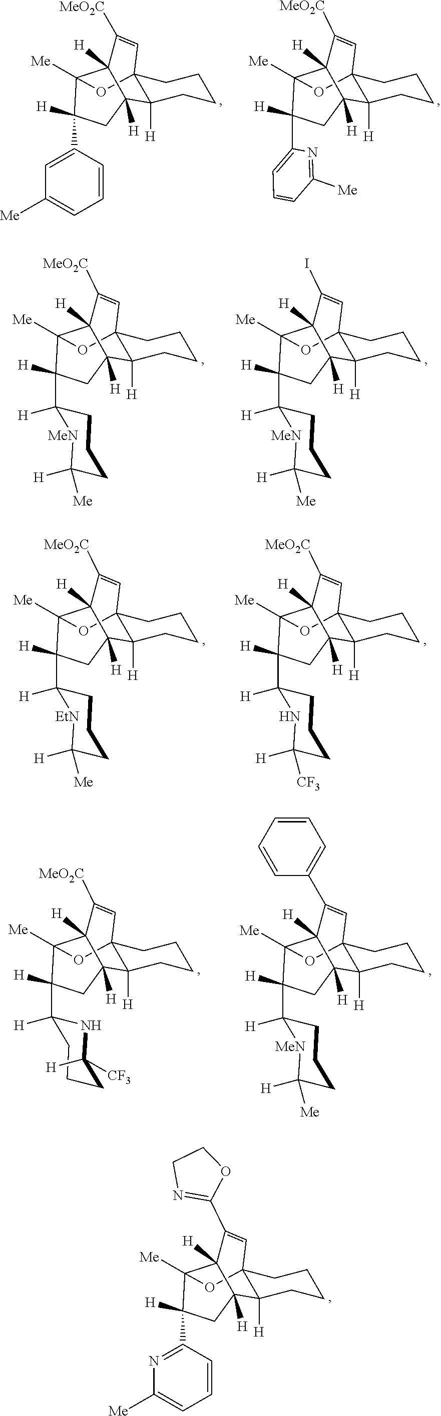 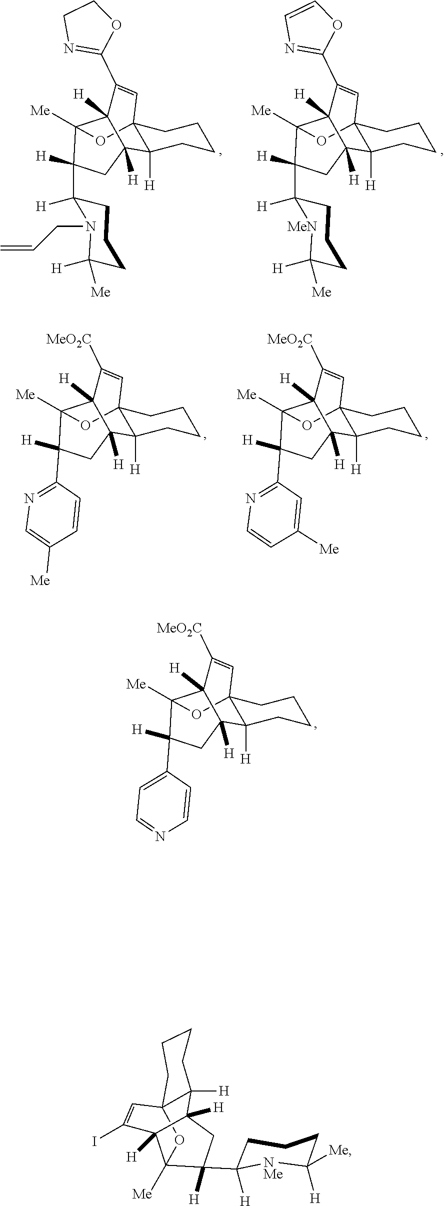 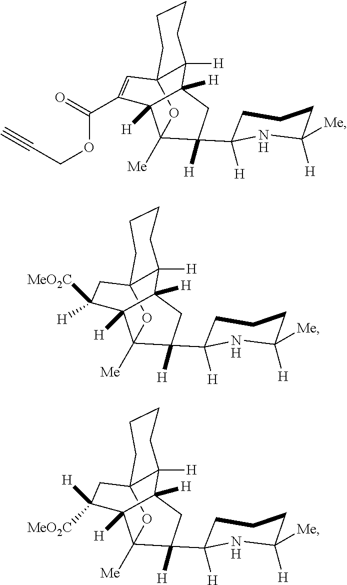 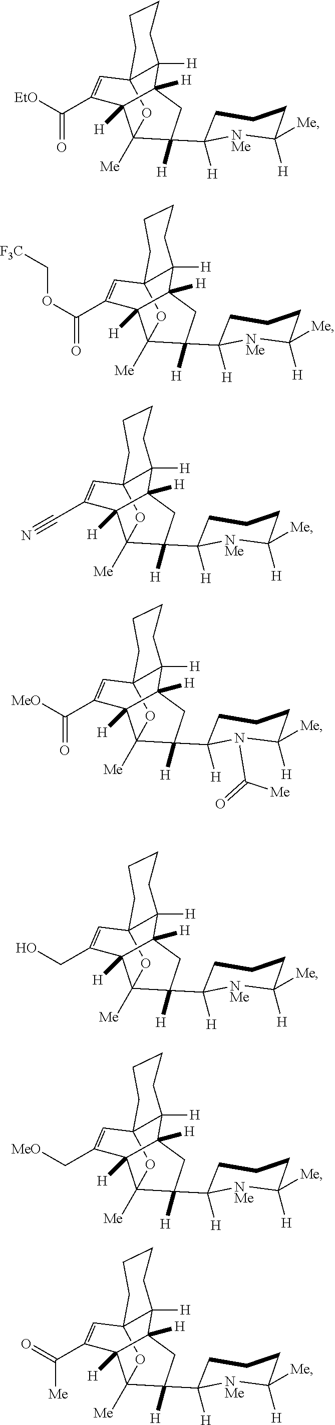 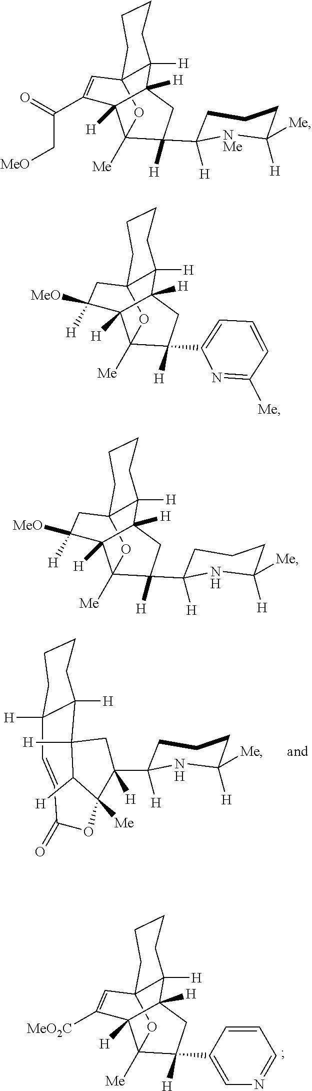
or a pharmaceutically acceptable salt thereof.

35. The method according to any one of claims 31-33, wherein the opioid receptor is a mu-opioid receptor (MOR), a kappa-opioid receptor (KOR) or a delta-opioid receptor (DOR).

36. The method according to claim 35, wherein the opioid receptor is a KOR or MOR.

37. A method of treating a disorder selected from the group consisting of substance abuse disorder, major depressive disorder, resistant depression, and impulse control disorder in a subject in need of such treatment comprising administering to the subject a therapeutically effective amount of a compound of Formula (I) 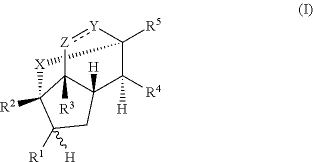
wherein: [symbol]
is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷;
Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇ cycloalkyl), —NMsPh, and —C(═O)OH;
R² and R³ are independently H, C₁-C₆ alkyl, (C₁-C₆ alkyl)-C(═O)—, hydroxy, or —O—(C₁-C₆ alkyl);
or R² and R³ together form C₃-C₇ cycloalkyl or C₃-C₇ heterocycloalkyl;
R⁴ and R⁵ are each independently H or C₁-C₆ alkyl;
or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
or R⁶ and R⁷ together form monocyclic or bicyclic C₃-C₁₄ cycloalkyl or C₃-C₁₄ heterocycloalkyl, each individually and optionally substituted with 1-3 R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl);
or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
each R^{p} independently is H, C₁-C₆ alkyl, (C₁-C₆ alkyl)-C(═O)—, —Si(C₁-C₆ alkyl)₃, —S(═O)₂—(C₁-C₆ alkyl), or —S(═O)₂—(C₆-C₁₀ aryl);
R^{q}, R^{s}, and R^{t} are each independently H or C₁-C₆ alkyl;
wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
or a pharmaceutically acceptable salt thereof.

38. The method according to claim 37, wherein, in Formula (I): [symbol]
is a double bond;
X is O;
Y is CR⁶;
Z is CR⁷;
R¹ is C₆-C₁₀ aryl or C₅-C₁₀ heteroaryl, optionally substituted with 1-3 substituents independently selected from the group consisting of C₁-C₆ alkyl, halo, —OH, —O—C₁-C₆ alkyl, —CN, —NO₂, —CF₃, methylene cyclopropyl, and —C(═O)OH;
R² is C₁-C₆ alkyl;
R³ is H;
R⁴ and R⁵ together with the carbon atoms to which they are shown attached form a C₃-C₇ cycloalkyl, which is optionally substituted with 1-2 substitutents independently selected from the group consisting of C₁-C₆ alkyl, halo, —OH, —O—C₁-C₆ alkyl, —CN, —NO₂, —CF₃, and —C(═O)OH;
R⁶ is H; and
R⁷ is —C(═O)—(C₁-C₆ alkyl).

39. The method according to claim 37 or 38, wherein the compound of Formula (I) or a pharmaceutically acceptable salt thereof is the compound of Formula (2): 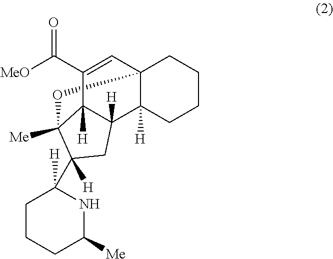 [SMILES: COC(=O)C1=C[C@@]23CCCC[C@H]2[C@@H]2C[C@H]([C@@H]4CCC[C@H](C)N4)[C@](C)(O3)[C@H]12]
or a pharmaceutically acceptable salt thereof.

40. The method according to claim 37, wherein the compound of Formula (I) or a pharmaceutically acceptable salt thereof is selected from the group consisting of: 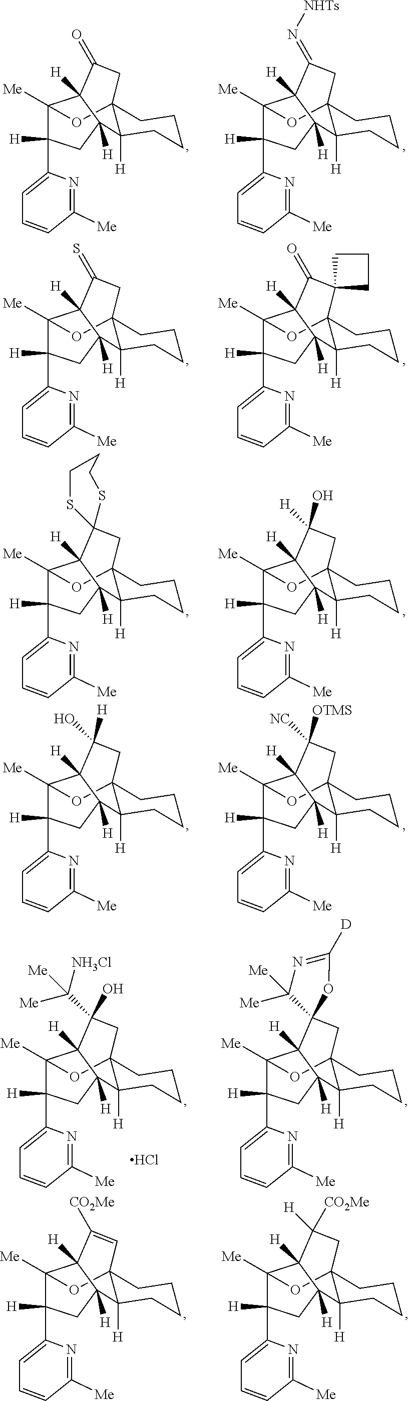 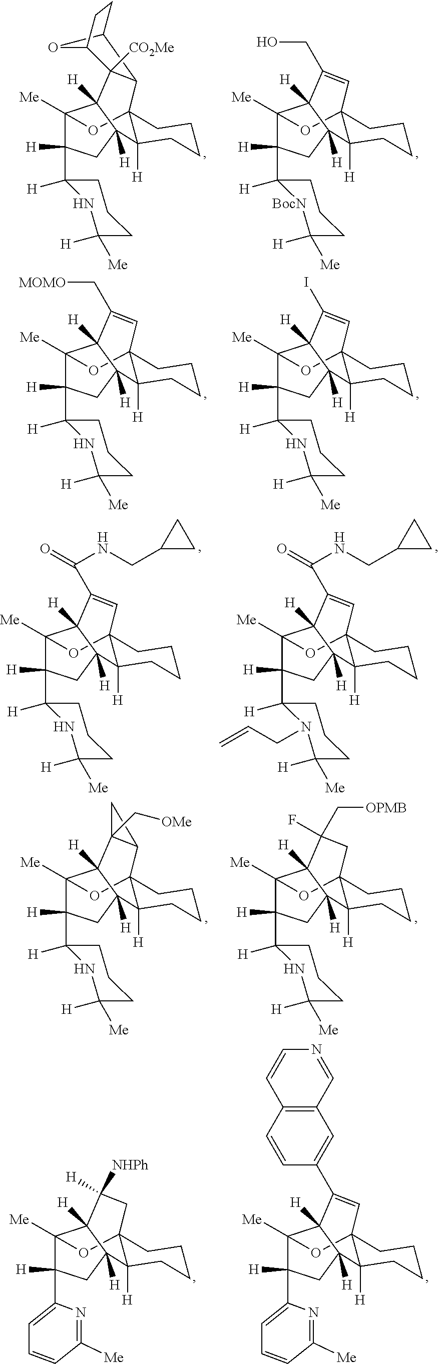 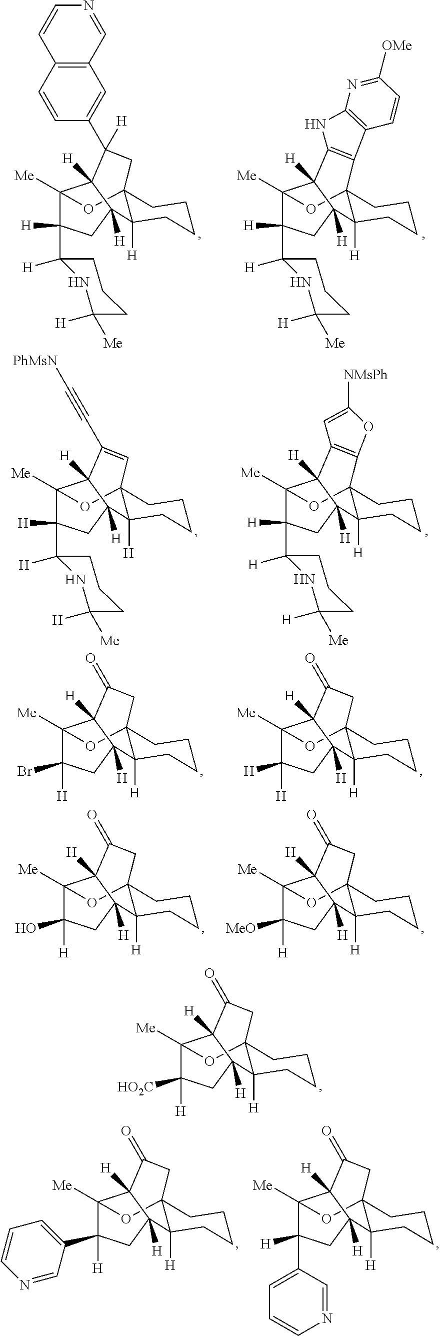 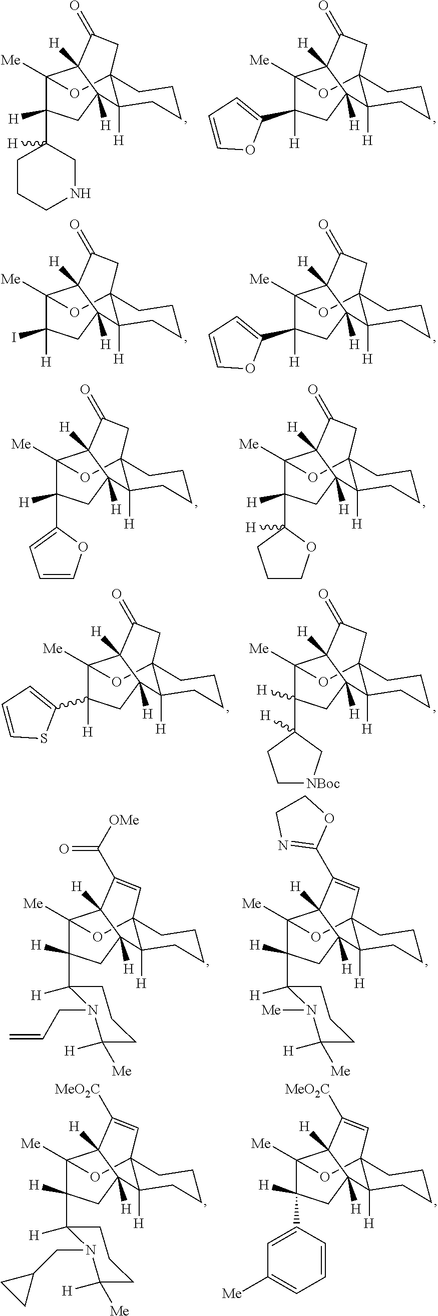 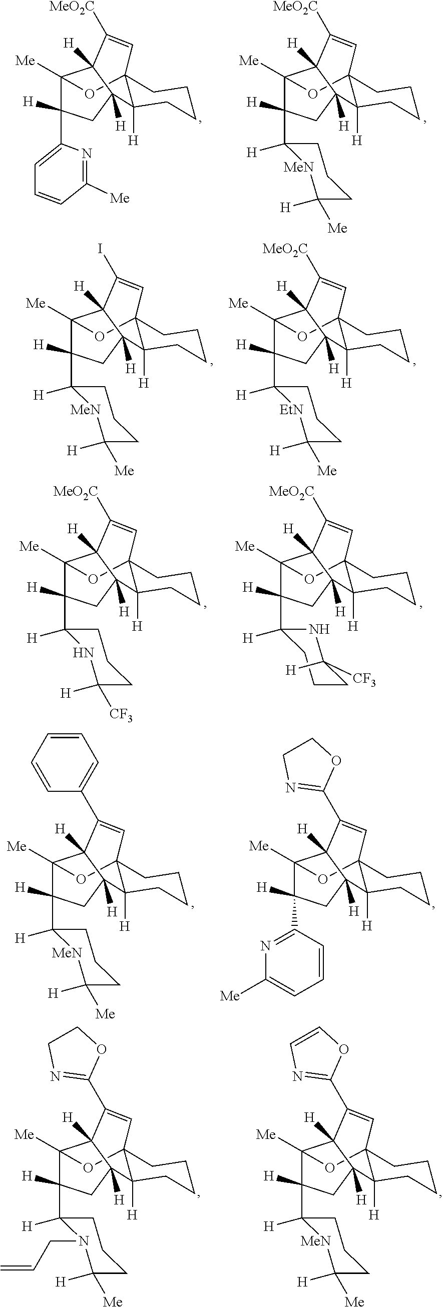 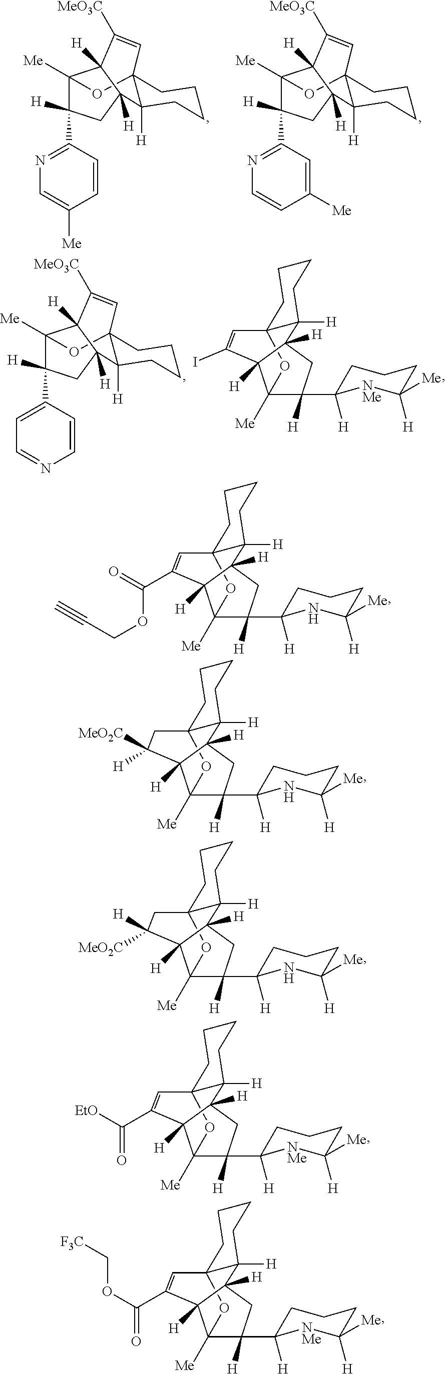 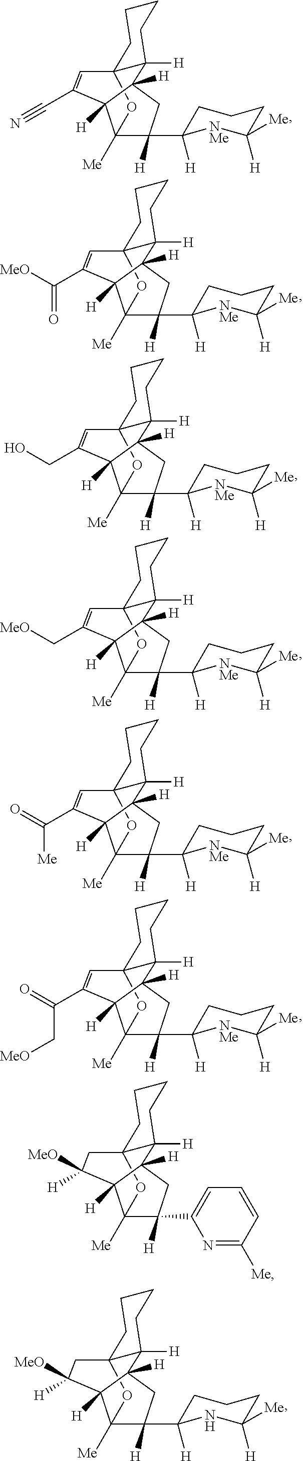 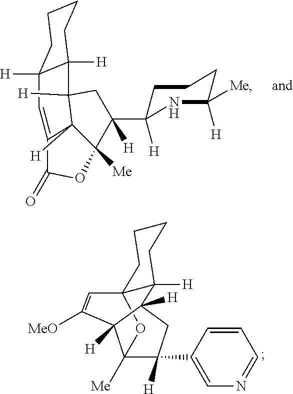
or a pharmaceutically acceptable salt thereof.

41. A method of agonizing an opioid receptor in a subject in need of such agonization, comprising administering to the subject a therapeutically effective amount of the compound of Formula (II): 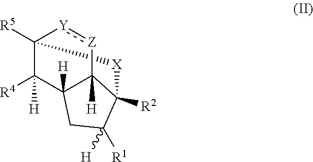
wherein: [symbol]
is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷;
Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇cycloalkyl), —NMsPh, and —C(═O)OH;
R² is H, C₁-C₆ alkyl, (C₁-C₆ alkyl)-C(═O)—, hydroxy, or —O—(C₁-C₆ alkyl);
R⁴ and R⁵ are each independently H or C₁-C₆ alkyl;
or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
or R⁶ and R⁷ together form monocyclic or bicyclic C₃-C₁₄ cycloalkyl or C₃-C₁₄ heterocycloalkyl, each individually and optionally substituted with 1-3 R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl);
or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
each R^{p} independently is H, C₁-C₆ alkyl, (C₁-C₆ alkyl)-C(═O)—, —Si(C₁-C₆ alkyl)₃, —S(═O)₂—(C₁-C₆ alkyl), or —S(═O)₂—(C₆-C₁₀ aryl);
R^{q}, R^{s}, and R^{t} are each independently H or C₁-C₆ alkyl;
wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
or a pharmaceutically acceptable salt thereof.

42. The method according to claim 41, wherein, in Formula (II): [symbol]
is a double bond;
X is O;
Y is CR⁶;
Z is CR⁷;
R¹ is C₆-C₁₀ aryl or C₅-C₁₀ heteroaryl, optionally substituted with 1-3 substituents independently selected from the group consisting of C₁-C₆ alkyl, halo, —OH, —O—C₁-C₆ alkyl, —CN, —NO₂, —CF₃, methylene cyclopropyl, and —C(═O)OH;
R² is C₁-C₆ alkyl;
R⁴ and R⁵ together with the carbon atoms to which they are shown attached form a C₃-C₇ cycloalkyl, which is optionally substituted with 1-2 substitutents independently selected from the group consisting of C₁-C₆ alkyl, halo, —OH, —O—C₁-C₆ alkyl, —CN, —NO₂, —CF₃, and —C(═O)OH;
R⁶ is H; and
R⁷ is —C(═O)—(C₁-C₆ alkyl).

43. The method of according to claim 41 or 42, wherein the compound of Formula (II) or a pharmaceutically acceptable salt thereof is the compound of Formula (1): 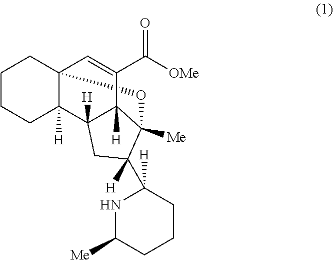 [SMILES: COC(=O)C1=C[C@]23CCCC[C@@H]2[C@H]2C[C@@H]([C@H]4CCC[C@@H](C)N4)[C@@](C)(O3)[C@@H]12]
or a pharmaceutically acceptable salt thereof.

44. The method according to any one of claims 41-43, wherein the opioid receptor is a kappa-opioid receptor (KOR).

45. A method of treating pain, itching, depression or dissociative hallucination in a subject in need of such treatment comprising administering to the subject a therapeutically effective amount of the compound of Formula (II): 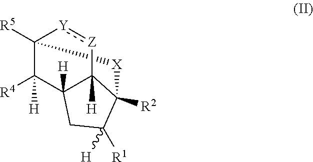
wherein: [symbol]
is a single or double bond;
X is —CH₂—, —O—, or —NR^{p};
Y is C(R⁶)(R^{6a}) with the proviso that when [symbol] is a single bond, and CR⁶ when [symbol] is a double bond;
Z is C=Q, CR⁷, or C(R^{7a})(R^{7b}) with the proviso that when [symbol] is a double bond, Z is CR⁷;
or R⁶ and R⁷ together form monocyclic or bicyclic C₃-C₁₄ cycloalkyl or C₃-C₁₄ heterocycloalkyl, each individually and optionally substituted with one or more R when [symbol] is a single bond;
Q is O, S, N—R^{p}, or N—OR^{q};
R¹ is D, halo, hydroxy, —O—(C₁-C₆ alkyl), —C(═O)OH, C₆-C₁₀ aryl, C₁-C₆ alkyl, a C₃-C₇ heterocycloalkyl, or C₅-C₁₄ heteroaryl, wherein each alkyl, aryl, heterocyclyl, or heteroaryl is optionally substituted with 1-3 R;
each R is independently selected from the group consisting of C₁-C₆ alkyl, halo, —OH, —O—C₁-C₆ alkyl, —CN, —NO₂, halo (C₁-C₆ alkyl), C₂-C₆ alkenyl, CH₂CH═CH₂, (C₁-C₆ alkyl)(C₃-C₇ cycloalkyl), —NMsPh, and —C(═O)OH;
R² is H, C₁-C₆ alkyl, (C₁-C₆ alkyl)-C(═O)—, hydroxy, or —O—(C₁-C₆ alkyl);
R⁴ and R⁵ are each independently H or C₁-C₆ alkyl;
or R⁴ and R⁵ together form C₃-C₇ cycloalkyl or C₃-C₇ heterocycloalkyl;
R⁶ and R^{6a} are each independently H, C₁-C₆ alkyl, —C(═O)—(C₁-C₆ alkyl), —C(═O)—O—(C₁-C₆ alkyl), —OH, -hydroxy C₁-C₆ alkyl, or —O—(C₁-C₆ alkyl);
or R⁶ and R^{6a} together form a C₃-C₇ cycloalkyl, mono or bicyclic C₃-C₁₄ heterocycloalkyl, or mono or bicyclic C₅-C₁₄ heteroaryl, each optionally substituted with one or more R;
or R⁵ and R⁶ are together form C₆-C₁₀ aryl, C₃-C₇ cycloalkyl, C₃-C₇ heterocycloalkyl, or C₅-C₁₄ heteroaryl;
R⁷ is H, halo, —CN, —CF₃, —OTMS, —O—S(═O)₂—CF₃, —O—S(═O)₂—CH₃, or —O—S(═O)₂-p-toluyl;
or R⁷ is C₁-C₆ alkyl, C₁-C₆ heteroalkyl, -halo C₁-C₆ alkyl, -halo C₁-C₆ heteroalkyl, -hydroxy C₁-C₆ alkyl, C₂-C₆ alkenyl, C₂-C₆ heteroalkenyl, halo C₂-C₆ alkenyl, halo C₂-C₆ heteroalkenyl, C₂-C₆ alkynyl, C₂-C₆ heteroalkynyl, —(C₁-C₆ alkyl)-O—(C₁-C₆ alkyl)-O—(C₁-C₆ alkyl), —O—S(═O)₂—CF₂)₃—CF₃, —C(═O)—(C₁-C₆ alkyl), —C(═O)—O—(C₁-C₆ alkyl), —C(═O)—O-(halo C₁-C₆ alkyl), —C(═O)—O—(C₁-C₆ heteroalkyl), —C(═O)—O—(C₂-C₆ alkenyl), —C(═O)—O—(C₂-C₆ alkynyl), —C₁-C₆ alkyl-(C₃-C₇cycloalkyl), —C(═O)—NH—(C₁-C₆ alkyl)(C₃-C₇cycloalkyl), —(C₁-C₆ alkyl)-OPMB, —NHPh, C₅-C₁₄ heteroaryl, C₆-C₁₀ aryl, C₅-C₁₀ heteroaryl, C₂-C₆ alkynyl, —C≡C—NMsPh, or each individually and optionally substituted with one or more R;
or R⁶ and R⁷ together form monocyclic or bicyclic C₃-C₁₄ cycloalkyl or C₃-C₁₄ heterocycloalkyl, each individually and optionally substituted with 1-3 R;
R^{7a} and R^{7b} are each independently H, halo, hydroxy, or C₁-C₆ alkyl which is optionally substituted with 1-3 substitutents selected from the group consisting of halo, hydroxy, —O—(C₁-C₆ alkyl), —O-p-methyoxbenzyl, —NH₂, and —NH₂Cl, cyano, —N(H)(C₆-C₁₀ aryl), —O—S(═O)₂—(C₁-C₆ alkyl), —O—S(═O)₂—(C₆-C₁₀ aryl), —C(═O)—O—(C₁-C₆ alkyl), (C₁-C₆ alkyl)-C(═O)—, —C(═O)—N(R^{q})(—(C₁-C₆ alkyl)-(C₃-C₇ cycloalkyl), —O—(C₁-C₆ alkyl), or —S—O—(C₁-C₆ alkyl); or R^{7a} and R^{7b} together form C₃-C₇ cycloalkyl, C₃-C₇ heterocycloalkyl, C₆-C₁₀ aryl or C₅-C₁₄ heteroaryl each individually and optionally substituted with one or more R;
each R^{p} independently is H, C₁-C₆ alkyl, —C(═O)—(C₁-C₆ alkyl), —Si(C₁-C₆ alkyl)₃, —S(═O)₂—(C₁-C₆ alkyl), —S(═O)₂—(C₆-C₁₀ aryl), or —NHTs;
R^{q}, R^{s}, and R^{t} are each independently H or C₁-C₆ alkyl;
wherein each instance of C₁-C₆ alkyl, halo C₁-C₆ alkyl, C₆-C₁₀ aryl, C₃-C₇ cycloalkyl, C₃-C₇ heterocycloalkyl, C₁-C₆ heteroalkyl, and C₅-C₁₄ heteroaryl are optionally independently substituted with 1-3 substituents independently selected from the group consisting of R, C₁-C₆ alkyl, halo, D, —OH, —O—(C₁-C₆ alkyl), —C₂-C₆ alkenyl, —N(—S(═O)₂—(C₁-C₆ alkyl)(C₆-C₁₀ aryl), —CN, —NO₂, —C(═O)OH, and —C(═O)—(C₁-C₆ alkyl);
wherein each instance of two adjacent carbon atoms of C₆-C₁₀ aryl, C₃-C₇ cycloalkyl, C₃-C₇ cycloalkyl, or C₅-C₁₄ heteroaryl may optionally form C₃-C₇ cycloalkyl, C₆-C₁₀ aryl, C₅-C₁₀ heteroaryl, or C₃-C₇ heterocycloalkyl, each may be individually and optionally substituted with one or more R; and
wherein each instance of C₅-C₁₄ heteroaryl may be unsaturated or partially unsaturated;
or a pharmaceutically acceptable salt thereof.

46. The method according to claim 45, wherein, in Formula (II): [symbol]
is a double bond;
X is O;
Y is CR⁶;
Z is CR⁷;
R¹ is C₆-C₁₀ aryl or C₅-C₁₀ heteroaryl, optionally substituted with 1-3 substituents independently selected from the group consisting of C₁-C₆ alkyl, halo, —OH, —O—C₁-C₆ alkyl, —CN, —NO₂, —CF₃, methylene cyclopropyl, and —C(═O)OH;
R² is C₁-C₆ alkyl;
R⁴ and R⁵ together with the carbon atoms to which they are shown attached form a C₃-C₇ cycloalkyl, which is optionally substituted with 1-2 substitutents independently selected from the group consisting of C₁-C₆ alkyl, halo, —OH, —O—C₁-C₆ alkyl, —CN, —NO₂, —CF₃, and —C(═O)OH;
R⁶ is H; and
R⁷ is —C(═O)—(C₁-C₆ alkyl).

47. The method according to claim 45 or 46, wherein the compound of Formula (II) or a pharmaceutically acceptable salt thereof is the compound of Formula (1): 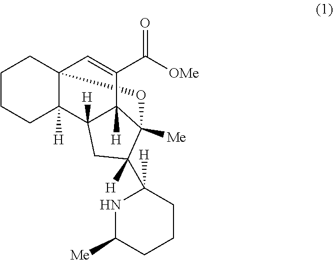 [SMILES: COC(=O)C1=C[C@]23CCCC[C@@H]2[C@H]2C[C@@H]([C@H]4CCC[C@@H](C)N4)[C@@](C)(O3)[C@@H]12]
or a pharmaceutically acceptable salt thereof.

48. A compound of formula I, having the structure of any one of the group consisting of: 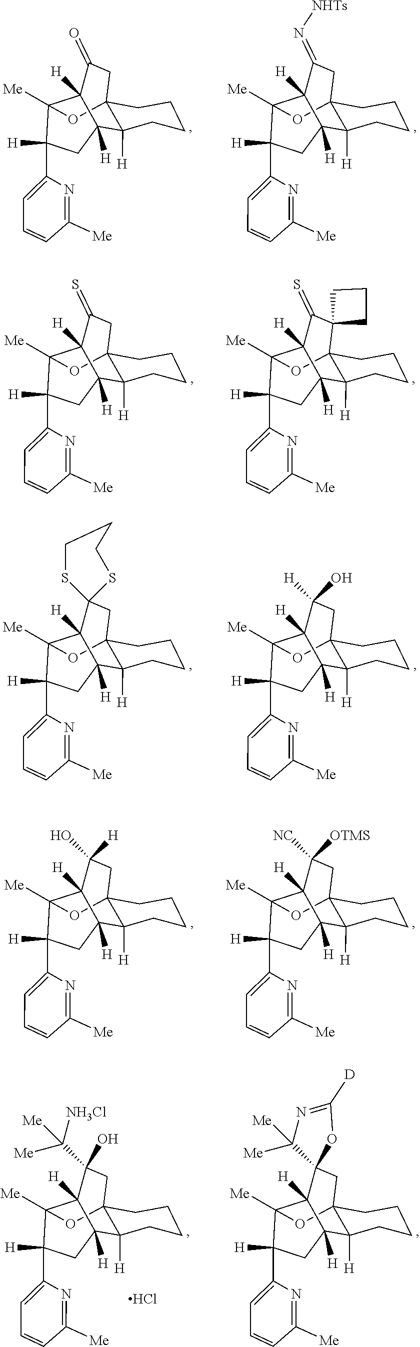 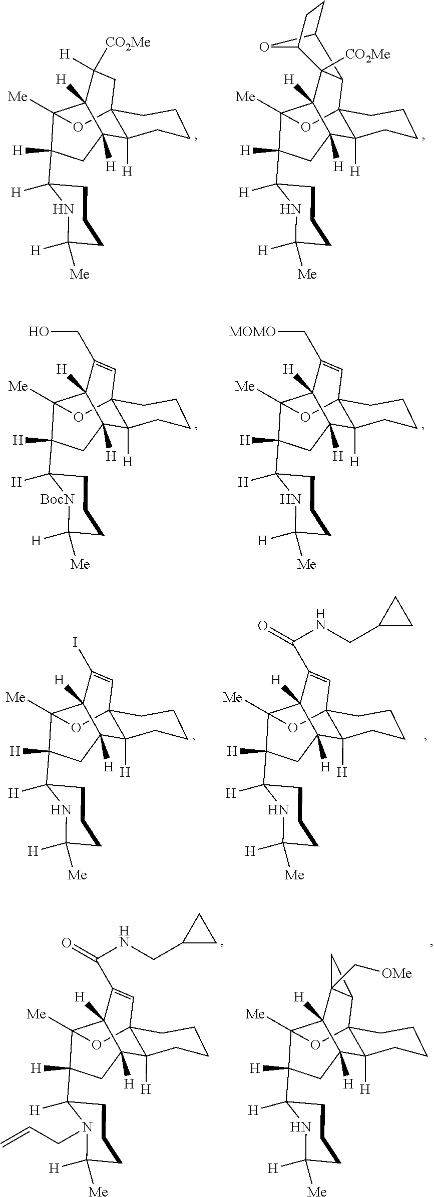 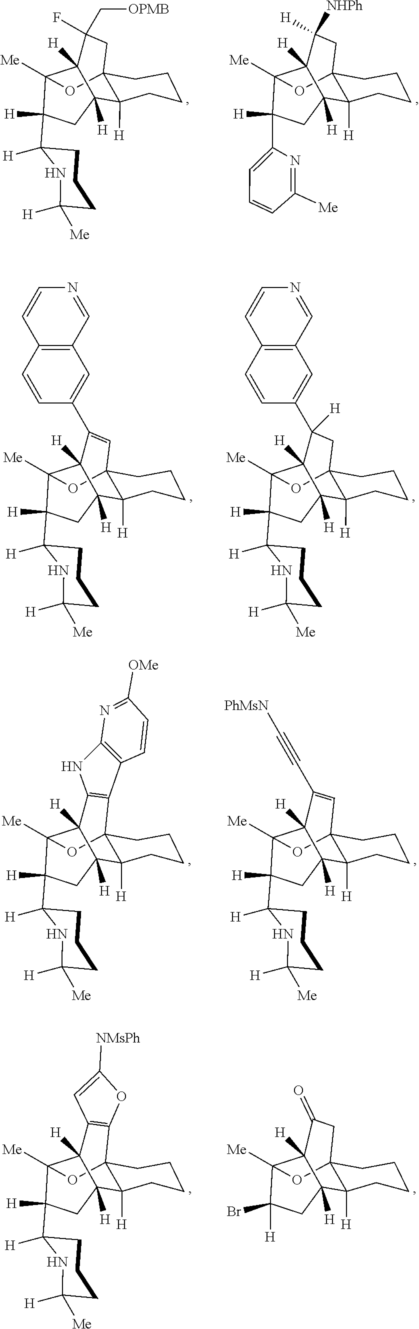 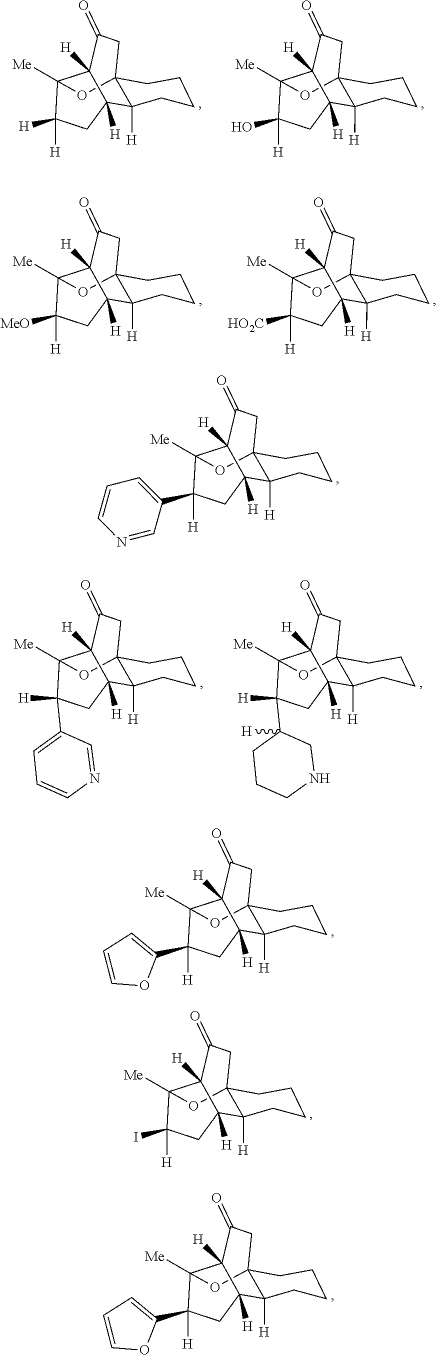 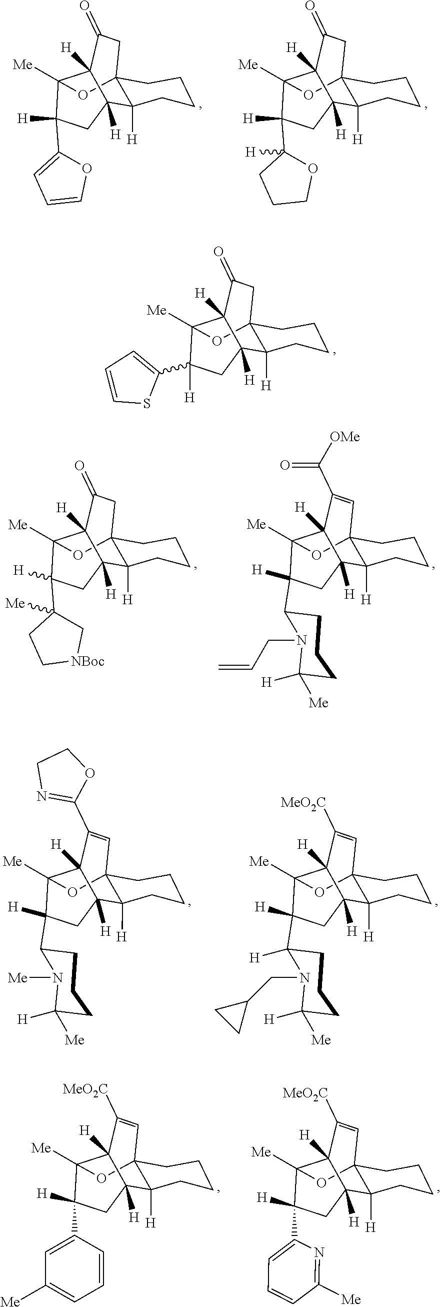 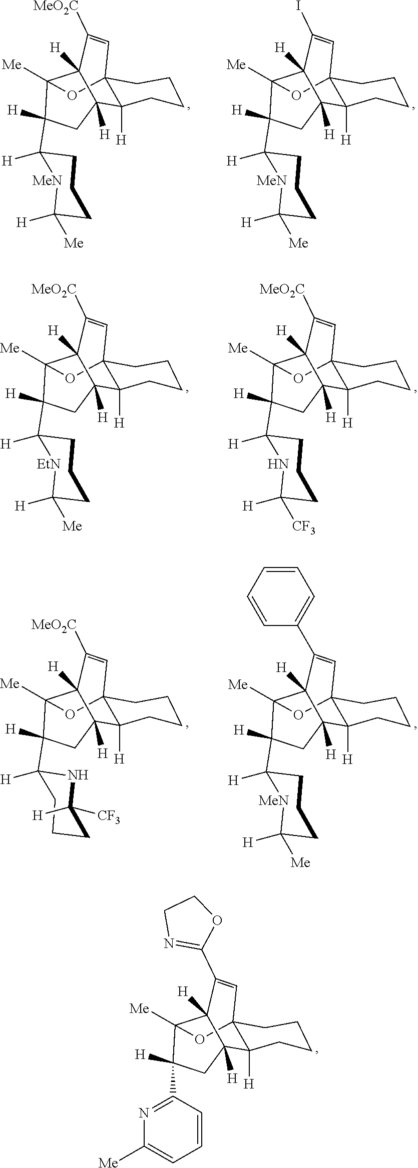 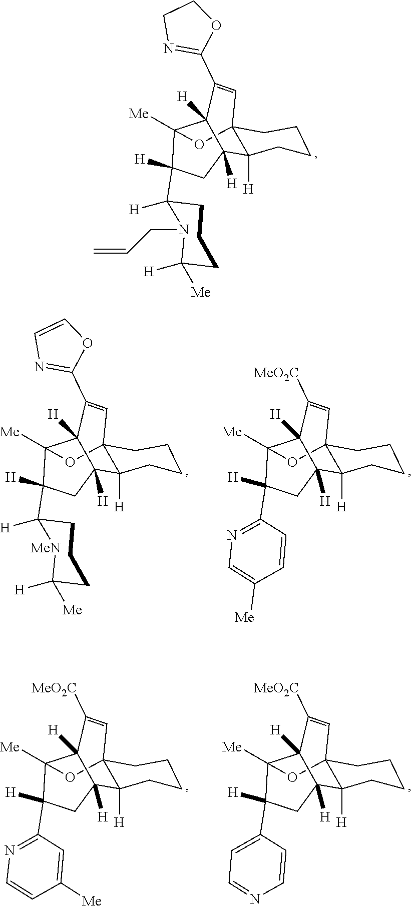 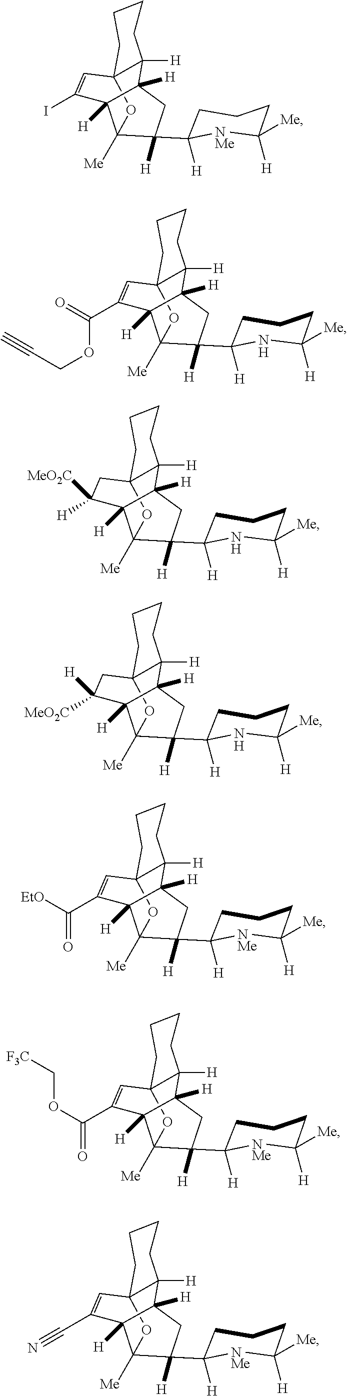 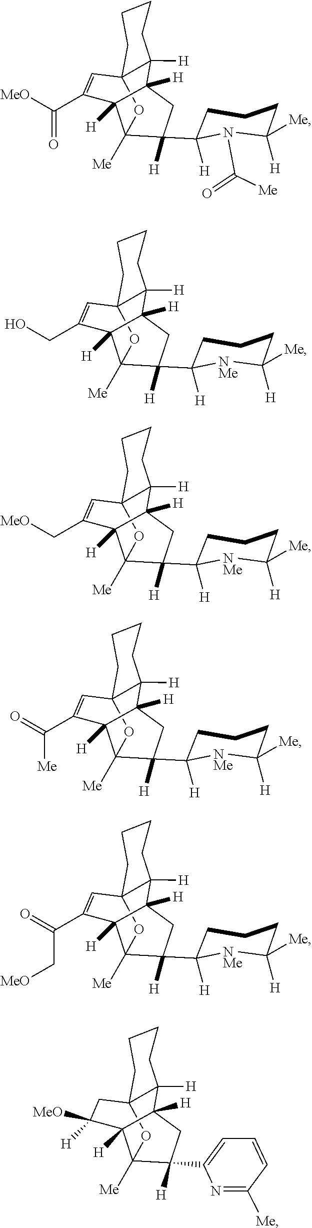 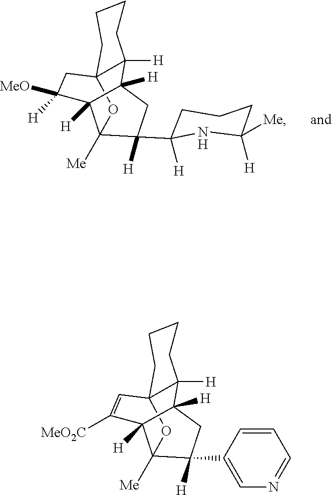 [SMILES: CO[C@H]1CC23CCCCC2[C@@H]2C[C@H](C4CCCC(C)N4)C(C)(O3)[C@H]12 ; "and" ; COC(=O)C1=CC23CCCCC2[C@@H]2C[C@H](c4cccnc4)C(C)(O3)[C@H]12]
including any enantiomers, scalemic or racemic mixtures, and pharmaceutically acceptable salts thereof.

49. A method of treating pain, itching, depression or dissociative hallucination in a subject in need of such treatment comprising administering to the subject a therapeutically effective amount of a compound of claim 48, including enantiomers, scalemic and racemic mixtures, and pharmaceutically acceptable salts thereof.

50. Any process, method or compound as disclosed herein.
